(19) United States
(12) Patent Application Publication
(10) Pub. No.: US 2024/0148037 A1
(43) Pub. Date: May 9, 2024

(54) STEVIOL GLYCOSIDE FORMULATIONS FOR FOOD AND BEVERAGES

(71) Applicant: SWEEGEN, INC., Rancho Santa Margarita, CA (US)

(72) Inventors: Steven Chen, Coto De Caza, CA (US); Shari MAHON, Laguna Hills, CA (US)

(73) Assignee: SWEEGEN, INC., Rancho Santa Margarita, CA (US)

(21) Appl. No.: 17/769,469

(22) PCT Filed: Oct. 16, 2020
(86) PCT No.: PCT/US2020/056030

§ 371 Date: Apr. 15, 2022

Related U.S. Application Data
(60) Provisional application No. 62/931,769, filed on Nov. 6, 2019.
(60) Provisional application No. 62/916,126, filed on Oct. 16, 2019.

(51) Int. Cl. A23L 27/30; A23G 9/32; A23K 20/163; A23L 2/60; C12P 17/06

(52) U.S. Cl. CPC A23L 27/36; A23G 9/32; A23K 20/163; A23L 2/60; C12P 17/06

(57) ABSTRACT

The present disclosure relates, at least in part to, the improvement of beverages and food items through utilization of rebaudioside blends that can provide optimal sweetness and taste. These sweetener formulations are similar in taste to sucrose and can allow for up to a 100% reduction in sucrose and other caloric sugars in food and beverage products.

### RELATED APPLICATIONS

This Application claims the benefit under 35 U.S.C. § 119(e) of U.S. Provisional Application No. 62/916,126, entitled “STEVIOL GLYCOSIDE FORMULATIONS FOR FOOD AND BEVERAGES,” filed on Oct. 16, 2019, and U.S. Provisional Application No. 62/931,769, entitled “STEVIOL GLYCOSIDE FORMULATIONS FOR FOOD AND BEVERAGES,” filed on Nov. 6, 2019, the entire contents of each of which are incorporated herein by reference.

### FIELD OF THE INVENTION

The present disclosure relates, at least in part, to the use of low or non-caloric sweetener compositions based on steviol glycosides, such as those derived from natural sources, as well as edible foods, including beverages, with such sweetener compositions. More specifically provided are particular formulations of rebaudiosides that elicit a pleasant sensory experience upon consumption.

### BACKGROUND

One of the main obstacles for the widespread use of Stevia sweeteners are their undesirable taste attributes. Alternative sweeteners and methods for their production are needed.

### SUMMARY

Many studies have focused on the connection of sugar consumption with obesity and other pathologies such as diabetes. Consumers and food companies alike are interested in calorie reduction through the use of sugar alternatives. There is also significant interest in the reduction of calories for companion animals or the use of sweeteners to make certain feed products more palatable.

Non-caloric natural and synthetic high-potency sweeteners often possess flavor profiles that are not as desirable to consumers as natural caloric sweeteners. Thus, it is desirable to develop improved non- or low-caloric sweeteners that can be substituted for sugar and that have a desirable taste profile. The species Stevia rebaudiana (“Stevia”) is the source of certain naturally occurring sweet steviol glycosides. Considerable research and development has focused on the use of sweet steviol glycosides of Stevia as non-caloric sweeteners, but there is a continuing interest in finding desirable mixes of Stevia-derived glycosides known as rebaudiosides, for use in a variety of food including beverage products.

Rebaudioside A is found to be between 150 and 320 times sweeter than sucrose, but has an anise or ‘licorice’ off-flavor that makes it among the least favorite compounds to use. Other desirable steviol glycosides include Rebaudioside D and Rebaudioside M, but each of the various steviol glycosides have their limitations in terms of taste, solubility or off-flavor and the optimal mix for use in various food products including beverages has not yet been found.

Methods for of the production of various rebaudioside molecules from Stevia and from Rebaudioside A, are described in, for example, U.S. Published Patent Applications 20170181452 and 20180037600, and U.S. Pat. Nos. 9,522,929, 10,010,099, 10,081,826, 10,253,344, all of which methods of production, including the compositions used and produced, are incorporated by reference herein. A need exists to provide a large-scale stable supply of the most desirable tasting Stevia rebaudiosides and to use those rebaudiosides in compositions that optimize the flavor of the food sweetened by them while restricting or controlling calorie content.

Surprisingly, steviol glycoside formulations that improve the solubility of the rebaudiosides used and lower the use of sucrose in food products while improving flavor and masking bitterness, have been found.

The present disclosure, in some aspects, provide steviol glycoside formulations containing a combination of rebaudiosides that provide a taste profile similar to sugar from onset of sweetness to sweetness linger, as determined through, e.g., the use of a panel of tasters for each of the formulations, by means of a sensory evaluation, as well as evaluation of its physical characteristics and capacity to replace a food/feed stuff made with a full or normal complement of sucrose. Where initial sensory testing of some blends in non-carbonated beverages detected slightly less sweet than full sugar product and some bitterness in aftertaste, the steviol glycoside formulations described herein were re-balanced to increase sweetness and reduce bitterness.

The present disclosure encompasses production of the steviol rebaudioside formulations to modify the taste perception of food products such that they, for example, exhibit enhanced sweetness, improved onset of sweetness, improved time and intensity of sweetness, and maskedbitterness and/or off notes.

The present disclosure relates, at least in part, to Stevia rebaudioside-based sweetener blends containing highly purified steviol glycosides. These blends include rebaudioside formulations comprising combinations and subsets of A, M, D, E, and I in varying quantities, and exhibit taste characteristics similar to sugar sweetener systems in, for example, carbonated (e.g., Cola and Non-Cola Carbonated) and non-carbonated beverages and concentrates, protein-based products, liquid dairy, yogurt, condiments, baked goods, jams, jellies and spreads. Additionally, the formulations can provide a higher solubility than the individual use of Rebaudioside M or Rebaudioside D with a taste profile closer to sucrose than individual rebaudiosides, such as specifically, Rebaudioside M or Rebaudioside D alone.

Therefore, further provided herein are the usage of the rebaudioside formulations in the production of food products, including beverages, for human consumption and feed production for animals and aquaculture. In some embodiments, the formulations contain multiple steviol glycosides, which comprises from about 0.1 wt. % to about 5.5 wt. %, preferably 1.0 wt. % to about 2.9 wt. % of the total food composition. In addition, other added elements in such food compositions can include erythritol and/or hydrocolloids such as pectin or gum Arabic.

Moreover, further provided herein are methods for optimizing food formulations to optimize health improvements in end consumers, in the form of a food item with a less dense calorie profile while retaining a desirable taste profile. This is also true for companion animals that may benefit from a calorie reduction in their daily diets. For animals produced for market the compositions provided herein can be used for changing the taste profile of lower quality feed or enhancing the flavor of feed containing nutrients that may be needed but that have bitterness or off-flavor.

In some embodiments, the rebaudioside blends provided allow for up to 100% sugar reduction in a wide-range of food products, including beverage products, with higher solubility characteristics than individual rebaudiosides and other known blends, while maintaining a desired flavor and taste profile and providing an onset of sweetness that is similar or almost identical to that of table sugar.

In some embodiments, the rebaudiosides may be produced by genetically modified microbes designed to produce sufficient quantities of steviol glycosides. It is apparent that this may be done with a much more limited geographic footprint than that needed for the production/breeding of Stevia rebaudiana plants.

Methods of improving the caloric profile of food for the elderly and the unwell, relative to the nutrients are also provided. In nutrition drinks designed for weight loss and/or nutrient delivery, the taste characteristics of the formulations provided are very similar to table sugar allowing for up to 100% sugar reduction in food products, including beverage products, while improving overall solubility and masking enhanced bitterness or off-flavors.

Further provided herein are commercially valuable processes for producing a low- or no-calorie composite sweetener composition comprising various Stevia rebaudiosides (See, e.g., FIG. 11) and uses thereof in various food products, including beverage and feed products.

Some aspects of the present disclosure provide steviol glycoside formulations consisting essentially of 40-60wt. % rebaudioside A (Reb A), 15-30 wt. % rebaudioside E (Reb E), 10-17 wt. % rebaudioside D (Reb D), and 5-10 wt. % rebaudioside M (Reb M).

Some aspects of the present disclosure provide steviol glycoside formulations consisting essentially of 40-60 wt. % rebaudioside A (Reb A), 15-30 wt. % rebaudioside E (Reb E), 10-17 wt. % rebaudioside D (Reb D), 5-10 wt. % rebaudioside M (Reb M), and 2-8 wt. % rebaudioside I (Reb I).

In some embodiments, Reb A is present in a concentration of 300-600 ppm, Reb E is present in a concentration of 50-200 ppm, Reb D is present in a concentration of 50-200 ppm, Reb M is present in a concentration of 200-500 ppm.

In some embodiments, Reb A is present in a concentration of 200-500 ppm, Reb E is present in a concentration of 50-300 ppm, Reb D is present in a concentration of 50-300 ppm, Reb M is present in a concentration of 5-100 ppm, and Reb I is present in a concentration of 5-50 ppm.

Other aspects of the present disclosure provide steviol glycoside formulations consisting essentially of rebaudioside A (Reb A), rebaudioside E (Reb E), rebaudioside D (Reb D), and rebaudioside M (Reb M), wherein Reb A is present in an amount of 300-600 ppm; Reb E is present in an amount of from 50-250 ppm; Reb D is present in an amount of 10-200 ppm; and/or Reb M is present in an amount of 10-150 ppm.

In some embodiments, the steviol glycoside formulation further comprises rebaudioside I (Reb I) in an amount of 1-50 ppm.

Further provided herein are steviol glycoside formulations consisting essentially of 500 ppm Reb A, 350 ppm Reb M, 100 ppm Reb D, and 100 ppm Reb E.

Further provided herein are steviol glycoside formulations consisting essentially of 373 ppm Reb A, 48 ppm Reb M, 100 ppm Reb D, 131 ppm Reb E, and 30 ppm Reb I.

In some embodiments, at least one rebaudioside is made by a genetically modified microbe.

Orally consumable product comprising the steviol glycoside formulation or the sweetener described herein are also provided.

In some embodiments, the orally consumable product is selected from the group consisting of a food composition, a beverage product, a dietary supplement, a nutraceutical, an edible gel mix, an edible gel composition, a pharmaceutical composition, a dental and oral hygiene composition, and an animal feed. In some embodiments, the orally consumable product is a dental and oral hygiene composition. In some embodiments, the dental and oral hygiene composition is a toothpaste.

In some embodiments, the steviol glycoside formulation is present in a concentration of 50-800 ppm. In some embodiments, the steviol glycoside formulation is present in the range of 0.0003% to 1.0% by weight of the total weight of the orally consumable product.

In some embodiments, the orally consumable product is a pharmaceutical composition. In some embodiments, the steviol glycoside formulation is present in a concentration of 50-800 ppm. In some embodiments, the steviol glycoside formulation is present in the range of 0.0004% to 1.25% by weight of the total weight of the orally consumable product.

In some embodiments, the orally consumable product is a beverage. In some embodiments, the beverage is a carbonated or non-carbonated beverage. In some embodiments, the beverage is selected from the group consisting of a soft drink, a fountain beverage, a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink. In some embodiments, the steviol glycoside formulation is present in a concentration of 65-800 ppm. In some embodiments, the steviol glycoside formulation is present in the range of 0.0005% to 1.0% by weight of the total weight of the orally consumable product.

In some embodiments, the orally consumable product is a food composition. In some embodiments, the food composition is selected from the group consisting of spreads, margarines, sports products, nutrition bars, infant formulas, mayonnaise, confectionary composition, a condiment, a chewing gum, a cereal composition, a baked good, a dairy product, and a tabletop sweetener composition. In some embodiments, the food composition is a yogurt. In some embodiments, the food composition is frozen. In some embodiments, the food composition is ice cream. In some embodiments, the steviol glycoside formulation is present in a concentration of 50-700 ppm. In some embodiments, the steviol glycoside formulation is present in the range of 0.0005% to 1.0% by weight of the total weight of the orally consumable product.

In some embodiments, the orally consumable product further comprises a component selected from the group consisting of sucrose, aroma compounds, flavoring compounds and mixtures thereof,

In some embodiments, the orally consumable product comprises tocopherols in an amount of at least 5 ppm.

In some embodiments, the orally consumable product further comprises at least one stabilizing agent selected from the group consisting of citric acid, sodium benzoate, t-butyl hydroquinone, ascorbyl palmitate, propyl gallate, and combinations thereof.

In some embodiments, the orally consumable product further comprises a moisture containing ingredient. In some embodiments, the moisture ingredient is an emulsion. In some embodiments, the orally consumable product further comprises a chelating agent.

In some embodiments, the orally consumable product is an animal feed product for livestock, companion animals and/or aquaculture. In some embodiments, the livestock is cattle, swine and/or poultry. In some embodiments, the steviol glycoside formulation is present in a concentration of 50-800 ppm. In some embodiments, the orally consumable product further comprises a hydrocolloid or erythritol.

Also provided herein are compositions in any one of the figures.

Further provided herein are methods for creating or enhancing a sweetening effect in an orally consumable product comprising adding an amount of the steviol glycoside formulation or the sweetener described herein sufficient to produce the desired degree of sweetness to the orally consumable product.

Further provided herein are sweeteners comprising rebaudioside I (Reb I) produced by a reaction mixture comprising a steviol glycoside; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of any one of SEQ ID NOs: 1-4.

In some embodiments, the reaction mixture further comprises a sucrose synthase comprising the amino acid sequence of SEQ ID NO: 8. In some embodiments, the steviol glycoside is rebaudioside A.

In some embodiments, the sweetener further comprises one or more steviol glycoside selected from the group consisting of: rebaudioside E (Reb E), rebaudioside A (Reb A), rebaudioside M (Reb M), and rebaudioside D (Reb D). In some embodiments, the sweetener further comprises Reb E, Reb A, Reb M, and Reb D.

In some embodiments, the Reb E is produced by a reaction mixture comprising stevioside, rebaudioside KA, or rubusoside; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of any one of SEQ ID NOs: 1, 5, and 7.

In some embodiments, the Reb A is produced by a reaction mixture comprising stevioside or rebaudioside D; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of SEQ ID NO: 1.

In some embodiments, the Reb M is produced by a reaction mixture comprising stevioside or rebaudioside D; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of SEQ ID NO: 1 or SEQ ID NO: 7.

In some embodiments, the Reb D is produced by a reaction mixture comprising rebaudioside A or rebaudioside E; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of any one of SEQ ID NOs: 1, 5, and 7.

In some embodiments, the reaction mixture further comprises a sucrose synthase comprising the amino acid sequence of SEQ ID NO: 8.

In some embodiments, the sweetener comprises 40-60 wt. % Reb A, 15-30 wt. % Reb E, 10-17 wt. % Reb D, 5-10 wt. % Reb M, and 2-8 wt. % Reb I.

In some embodiments, Reb A is present in a concentration of 200-500 ppm, Reb E is present in a concentration of 50-300 ppm, Reb D is present in a concentration of 50-300 ppm, Reb M is present in a concentration of 5-100 ppm, and Reb I is present in a concentration of 5-50 ppm.

Other features and advantages of this disclosure will become apparent in the following detailed description of preferred embodiments of this disclosure, taken with reference to the accompanying figures, or will otherwise be apparent to one or ordinary skill in the art.

### BRIEF DESCRIPTION OF THE DRAWINGS

FIGS. 1A-1D depict the use for a lemon water, respectively, of sucrose, Reb M, Reb D and the formulations comprising Reb M, Reb D, Reb E, Reb A and Reb I (Blend 2), FIGS. 1B-1D representing a 100% sugar reduction in non-carbonated beverages including both liquid and dry concentrates.

FIGS. 2A-2D depict the use, respectively, of sucrose, Reb M, Reb D and the present formulations comprising Reb M, Reb D, Reb E, Reb A and Reb I (FIG. 2D, Blend 2) for cola, FIGS. 2B-2D representing up to a 100% sugar reduction in cola carbonates.

FIGS. 3A-3D depict the use, respectively, of sucrose, Reb M, Reb D and the present formulations comprising Reb M, Reb D, Reb E, Reb A and Reb I (FIG. 3D, Blend 2) for carbonated orange soda, FIGS. 3B-3D representing up to a 100% sugar reduction in a carbonated orange soda.

FIGS. 4A-4D depict the use, respectively, of sucrose, Reb M, Reb D and the present formulations comprising Reb M, Reb D, Reb E, Reb A and Reb I (FIG. 4D, Blend 2) for chocolate milk, FIGS. 4B-4D representing up to a 100% sugar reduction in a chocolate milk drink. Specifically, FIGS. 4B-4D show the use of the current formulations in chocolate milk providing up to a 100% sugar reduction in dairy applications (liquid and powdered).

FIGS. 5A-5D depict the use, respectively, of sucrose, Reb M, Reb D and the present formulations comprising Reb M, Reb D, Reb E, Reb A and Reb I (FIG. 5D, Blend 2) for chocolate almond milk, FIGS. 5B-5D representing up to a 100% sugar reduction in a chocolate milk drink. Specifically, FIGS. 5B-5D show the use of the current formulations in chocolate almond milk providing up to a 100% sugar reduction in dairy applications (liquid and powdered).

FIGS. 6A-6D depict the use, respectively, of sucrose, Reb M, Reb D and the present formulations comprising Reb M, Reb D, Reb E, Reb A and Reb I (FIG. 6D, Blend 2) for vanilla yogurt, FIGS. 6B-6D representing up to a 100% sugar reduction in vanilla yogurt. Specifically, FIGS. 6B-6D show the use of the current formulations in vanilla yogurt providing up to a 100% sugar reduction in yogurt (fruited and non-fruited).

FIGS. 7A-7D depict the use, respectively, of sucrose, Reb M, Reb D and the present formulations comprising Reb M, Reb D, Reb E, Reb A and Reb I (FIG. 7D, Blend 2) for chocolate almond milk, FIGS. 7B-7D representing up to a 100% sugar reduction in banana mini muffins. Specifically, FIGS. 7B-7D show the use of the current formulations in banana mini muffins providing up to a 100% sugar reduction in cakes, pastries, muffins, pies, breads and desserts.

FIGS. 8A-8D depict the use, respectively, of sucrose, Reb M, Reb D and the present formulations comprising Reb M, Reb D, Reb E, Reb A and Reb I (FIG. 8D, Blend 2) for vanilla butter cookies, FIGS. 8B-8D representing up to a 100% sugar reduction in vanilla butter cookies. Specifically, FIGS. 8B-8D show the use of the current formulations in banana mini muffins providing up to a 100% sugar reduction in cookies, crackers and snacks.

FIGS. 9A-9D depict the use, respectively, of sucrose, Reb M, Reb D and the present formulations comprising Reb M, Reb D, Reb E, Reb A and Reb I (FIG. 9D, Blend 2) in ketchup, FIGS. 9B-9D representing up to a 100% sugar reduction in vanilla butter cookies. Specifically, FIGS. 9B-9D show the use of the current formulations in ketchup providing up to a 100% sugar reduction in condiments.

FIGS. 10A-10D depict the use, respectively, of sucrose, Reb M, Reb D and the present formulations comprising Reb M, Reb D, Reb E, Reb A and Reb I (FIG. 10D, Blend 2) in peanut butter, FIGS. 10B-10D representing up to a 100% sugar reduction in peanut butter. Specifically, FIGS. 10B-10D show the use of the current formulations in ketchup providing up to a 100% sugar reduction in fruit preps, jams, jellies, nut butters and spreads.

FIGS. 11A-11B show exemplary formulations broken down into individual components and relative amounts.

FIG. 12 shows a process flow diagram for the production of soymilk.

FIG. 13 shows a process flow diagram for the production of margarine.

FIG. 14 shows the use of a sweetener system in companion animal feed comprising a rebaudioside blend comparable to up to a 100% sugar reduction in such feed.

FIG. 15 shows a full sugar lemon water composition and a zero-calorie lemon water composition containing Blend 2 described herein.

FIG. 16 shows a full sugar lemonade composition and a reduced calorie lemonade composition containing Blend 2 described herein.

FIG. 17 shows peach juice drinks with full sugar or reduced sugar (33% reduction or 50% reduction).

FIG. 18 shows a reduced sugar peach juice drink (80% reduction) and a zero-calorie peach juice drink containing Blend 2 described herein.

FIG. 19 shows lemonade compositions with full sugar or reduced sugar (33% reduction or 50% reduction).

FIG. 20 shows reduced sugar lemonade composition (80% reduction) and a zero-calorie lemonade composition containing Blend 2 described herein.

FIG. 21 shows a full sugar orange carbonated soft drink (CSD) composition and a zero-calorie orange CSD containing Blend 2 described herein.

FIG. 22 shows peach sparkling water with full sugar or reduced sugar (40% reduction).

FIG. 23 shows reduced peach sparkling water (80% reduction) and a zero-calorie peach sparkling water containing Blend 2 described herein.

FIG. 24 shows a full sugar hard lemonade composition and a reduced sugar hard lemonade containing Blend 2 described herein.

FIG. 25 shows a full sugar peach energy drink and a zero-calorie peach energy drink containing Blend 2 described herein.

FIG. 26 shows a full sugar peach energy drink and a zero-calorie peach energy drink containing Blend 2 described herein.

FIG. 27 shows a full sugar mango whey protein drink and a reduced sugar (60% reduction) mango whey protein drink containing Blend 2 described herein.

FIG. 28 shows a full sugar mango whey protein drink and a reduced sugar (60% reduction) mango whey protein drink containing Blend 2 described herein.

FIG. 29 shows a full sugar chocolate almond breeze composition, a reduced sugar (60% reduction) chocolate almond breeze composition containing Blend 2 described herein, and a 100% reduced sugar chocolate almond breeze composition containing Blend 2 described herein.

FIG. 30 shows a full sugar chocolate soymilk composition and a reduced sugar (60% reduction) chocolate soymilk composition containing Blend 2 described herein.

FIG. 31 shows a full sugar strawberry filing and a reduced sugar (80% reduction) strawberry filing containing Blend 2 described herein.

FIG. 32 shows a full sugar strawberry juice milk smoothie and a reduced sugar (60% reduction) strawberry juice milk smoothie containing Blend 2 described herein.

FIG. 33 shows a full sugar orange juice milk smoothie and a reduced sugar (80% reduction) orange juice milk smoothie containing Blend 2 described herein.

FIG. 34 shows a full sugar mango juice milk smoothie and a 100% reduced sugar mango juice milk smoothie containing Blend 2 described herein.

FIG. 35 shows a full sugar chocolate milk and a reduced sugar (60% reduction) chocolate milk containing Blend 2 described herein.

FIG. 36 shows a full sugar chocolate sauce and a reduced sugar (60% reduction) chocolate sauce containing Blend 2 described herein.

FIG. 37 shows a full sugar vanilla yogurt and a reduced sugar (80% reduction) vanilla yogurt containing Blend 2 described herein.

FIG. 38 shows a full sugar vanilla pea protein yogurt and a reduced sugar (80% reduction) vanilla pea protein yogurt containing Blend 2 described herein.

FIG. 39 shows a full sugar vanilla ice cream and a reduced sugar (80% reduction) vanilla ice cream containing Blend 2 described herein.

FIG. 40 shows a full sugar soy vanilla ice cream and a reduced sugar (80% reduction) soy vanilla ice cream containing Blend 2 described herein.

FIG. 41 shows a full sugar mango sherbet and a 100% reduced sugar mango sherbet containing Blend 2 described herein.

FIG. 42 shows a full sugar pea protein mango sherbet and a 100% reduced sugar pea protein mango sherbet containing Blend 2 described herein.

FIG. 43 shows a full sugar vanilla butter cookie composition and a reduced sugar (80% reduction) vanilla butter cookie composition containing Blend 2 described herein.

FIG. 44 shows a full sugar banana mini muffin composition and a reduced sugar (80% reduction) banana mini muffin composition containing Blend 2 described herein.

FIG. 45 shows a full sugar cranberry granola bar composition and a 100% reduced sugar cranberry granola bar composition containing Blend 2 described herein.

FIG. 46 shows a full sugar cinnamon granola crunch composition and a 100% reduced sugar cinnamon granola crunch composition containing Blend 2 described herein.

FIG. 47 shows a full sugar tomato ketchup composition, a reduced sugar (50% reduction) tomato ketchup composition containing Blend 2 described herein, and a 100% reduced sugar tomato ketchup composition containing Blend 2 described herein.

FIG. 48 shows a full sugar creamy French dressing composition, a reduced sugar (50% reduction) creamy French dressing composition containing Blend 2 described herein, and a 100% reduced sugar creamy French dressing composition containing Blend 2 described herein.

FIG. 49 shows a full sugar tomato ketchup composition and a 100% reduced sugar tomato ketchup composition containing Blend 2 described herein.

FIG. 50 shows a full sugar creamy French dressing composition and a 100% reduced sugar creamy French dressing composition containing Blend 2 described herein.

FIG. 51 shows a full sugar BBQ sauce composition and a 100% reduced sugar BBQ sauce composition containing Blend 2 described herein.

### DETAILED DESCRIPTION

### Definitions

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

As used herein, “synthetic” or “organically synthesized” or “chemically synthesized” or “organically synthesizing” or “chemically synthesizing” or “organic synthesis” or “chemical synthesis” are used to refer to preparing the compounds through a series of chemical reactions; this does not include extracting the compound, for example, from a natural source.

The term “orally consumable product” as used herein refers to any beverage, food product, dietary supplement, nutraceutical, pharmaceutical composition, dental hygienic composition and cosmetic product which are contacted with the mouth of man or animal, including substances that are taken into and subsequently ejected from the mouth and substances which are drunk, eaten, swallowed, or otherwise ingested; and that are considered safe for human or animal consumption when used in a generally acceptable range of concentrations.

The term “food product” or “food composition” as used herein includes fruits, vegetables, juices, meat products such as ham, bacon and sausage; egg products, fruit concentrates, gelatins and gelatin-like products such as jams, jellies, preserves, and the like; milk products such as ice cream, sour cream, yogurt, and sherbet; icings, syrups including molasses; corn, wheat, rye, soybean, oat, rice and barley products, cereal products, nut meats and nut products, cakes, cookies, confectionaries such as candies, gums, fruit flavored drops, and chocolates, chewing gum, mints, creams, icing, ice cream, pies and breads. “Food product” also refers to condiments such as herbs, spices and seasonings, flavor enhancers, such as monosodium glutamate. “Food product” further also includes prepared packaged products, such as dietetic sweeteners, liquid sweeteners, tabletop flavorings, granulated flavor mixes which upon reconstitution with water provide non-carbonated drinks, instant pudding mixes, instant coffee and tea, coffee whiteners, malted milk mixes, pet foods, livestock feed, tobacco, and materials for baking applications, such as powdered baking mixes for the preparation of breads, cookies, cakes, pancakes, donuts and the like. “Food product” also includes diet or low-calorie food and beverages containing little or no sucrose.

As used herein, the term “sweetness intensity” refers to the relative strength of sweet sensation as can be observed or experienced by an individual, e.g., a human, or a degree or amount of sweetness detected by a taster, for example on a Brix scale.

As used herein, the term “enhancing the sweetness” refers to the effect of rebaudiosides in increasing, augmenting, intensifying, accentuating, magnifying, and/or potentiating the sensory perception of one or more sweetness characteristics of an orally consumable product as provided herein as compared to a corresponding orally consumable product that does not contain the rebaudiosides.

As used herein, the term “off-taste(s)” refers to an amount or degree of taste that is not characteristically or usually found or expected in an orally consumable product. For example, an off-taste is an undesirable taste of a sweetened consumable, such as, a bitter taste, a licorice-like taste, a metallic taste, an aversive taste, an astringent taste, a delayed sweetness onset, a lingering sweet aftertaste, and the like, etc.

As used herein, the term “wt. %” refers to the weight % of a compound (e.g., a rebaudioside) relative to the total weight of all compounds (e.g., all rebaudiosides) in a composition, such as a steviol glycoside formulation.

As used herein, the term “ppm” refers to part(s) per million by weight, for example, the weight of a compound, such as rebaudioside V and/or rebaudioside W (in milligrams) per kilogram, of a composition, such as an orally consumable product, containing such compound (i.e., mg/kg) or the weight of a compound, such as rebaudioside V and/or rebaudioside W (in milligrams) per liter, of a composition, such as an orally consumable product, containing such compound (i.e., mg/L); or by volume, for example the volume of a compound, such as a rebaudioside (in milliliters) per liter, of a composition, such as an orally consumable product containing such compound (i.e., ml/L).

As used herein, the term “sweetness intensity” refers to the relative strength of a sweet sensation as can observed or experienced by an individual, e.g., a human, or a degree or amount of sweetness detected by a taster, for example on a Brix scale.

As used herein, the term “carbohydrate sweetener” includes caloric sweeteners, such as, sucrose, fructose, glucose, high fructose corn syrup (containing fructose and glucose), xylose, arabinose, rhamnose, and sugar alcohols, such as erythritol, xylitol, mannitol, sorbitol, and inositol.

As used herein, the term “flavoring” or the like refers to any food-grade material that may be added to or present in an orally consumable product to provide a desired flavor.

The term “isolated” is used according to its ordinary and customary meaning as understood by a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “recombinant,” “heterologous,” and “exogenous,” when used herein in connection with polynucleotides, are used according to their ordinary and customary meanings as understood by a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

As used herein, the singular form “a”, “an”, and “the” includes plural references unless indicated otherwise.

Reference to “about” a value or parameter herein refers to the usual error range for the respective value readily known to the skilled person in this technical field. Reference to “about” for a value or parameter herein includes (and describes) aspects that are directed to that value or parameter per se. For example, description referring to “about X” includes description of “X.”

### Steviol Glycosides and Methods of Producing

Steviol glycosides can be isolated from Stevia rebaudiana leaves. Steviol glycosides are used as high intensity, low-calorie sweeteners and are significantly sweeter than sucrose. As natural sweeteners, different steviol gly?cosides have different degrees of sweetness and after-taste. For example, stevioside is 100-150 times sweeter than sucrose with bitter after-taste. Rebaudioside C is between 40-60 times sweeter than sucrose. Dulcoside A is about 30 times sweeter than sucrose.

Naturally occurring steviol glycosides share the same basic steviol structure, but differ in the content of carbohydrate residues (e.g., glucose, rhamnose and xylose residues) at the C13 and C19 positions. Steviol glycosides with known structures include, steviol, stevioside, rebaudioside A, rebaudioside B, rebaudioside C, rebaudioside D, rebaudioside E, rebaudioside F, rebaudioside M, rebaudioside I, and dulcoside A. Structures of examples of steviol glycosides are provided in Table 1.

TABLE 1
Examples of Steviol Glycosides.

| | | Molecular | Molecular |
|---|---|---|---|
| Name | Structure | Formula | Weight |
| Steviol | 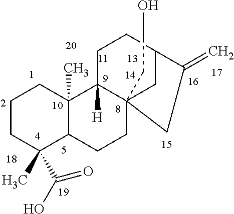 | C₂₀H₃₀O₃ | 318 |
| Stevioside | 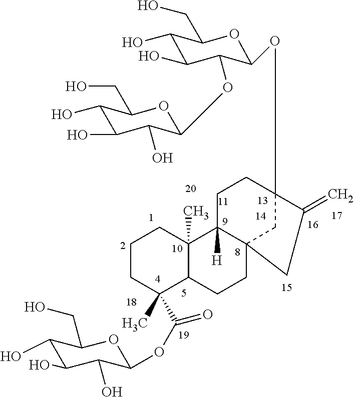 | C₃₈H₆₀O₁₈ | 804 |
| Rebaudioside A | 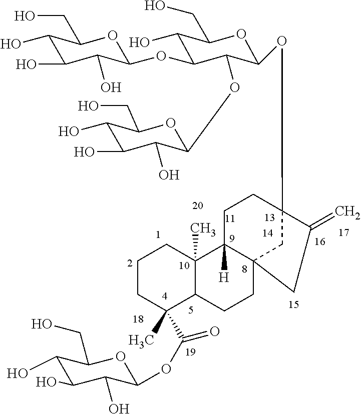 | C₄₄H₇₀O₂₃ | 966 |
| Rebaudioside D | 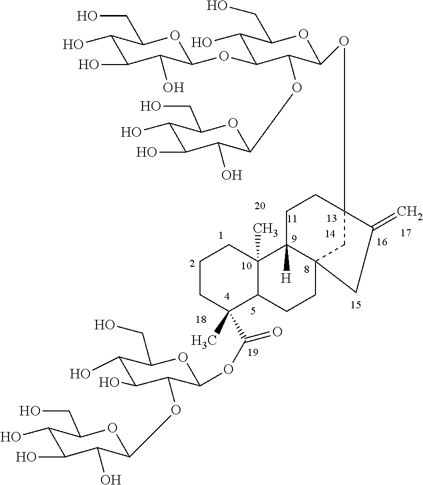 | C₅₀H₈₀O₂₈ | 1128 |
| Rebaudioside E | 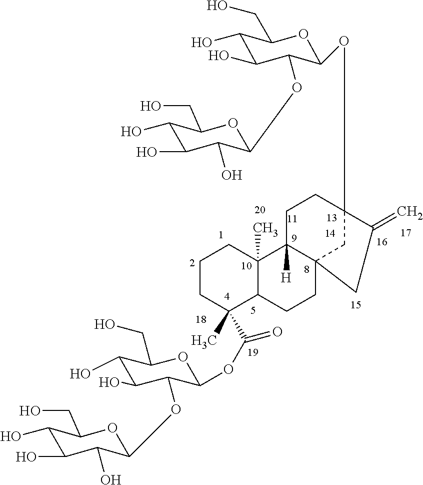 | C₄₄H₇₀O₂₃ | 966 |
| Rebaudioside M | 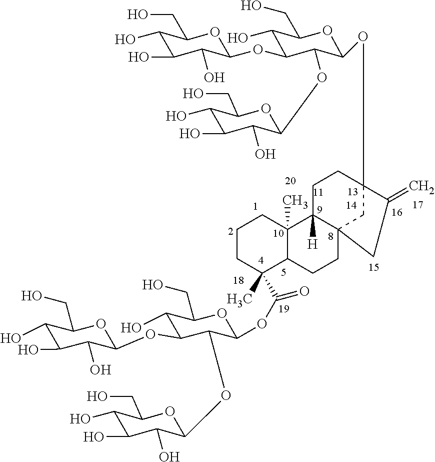 | C₅₆H₉₀O₃₃ | 1291.3 |
| Rebaudioside I | 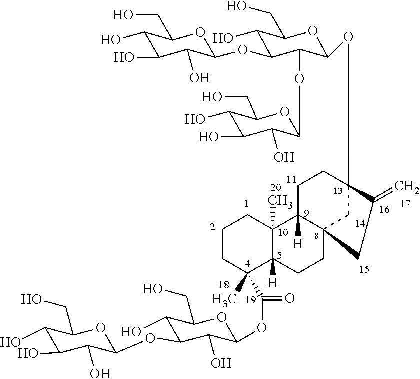 | C₅₀H₈₀O₂₈ | 1129.15 |
| Rebaudioside KA | 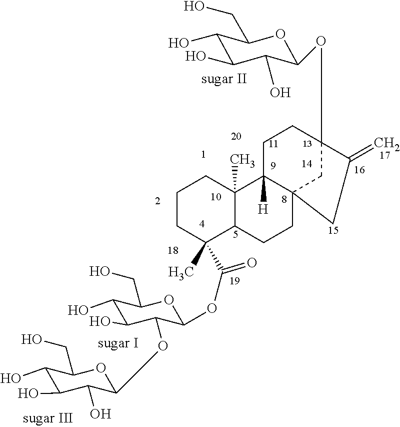 | C₃₈H₆₀O₁₈ | 804 |

The majority of steviol glycosides are formed by several glycosylation reactions of steviol, which are typically catalyzed by the UDP-glycosyltransferases (UGTs) using uridine 5′-diphosphoglucose (UDP-glucose) as a donor of the sugar moiety. UGTs in plants make up a very diverse group of enzymes that transfer a glucose residue from UDP-glucose to steviol. For example, glycosylation of the C-3′ of the C-13-O-glucose of stevioside yields rebaudioside A; and glycosylation of the C-2′ of the 19-O-glucose of the stevioside yields rebaudioside E. Further glycosylation of rebaudioside A (at C-2′-19-O-glucose) or rebaudioside E (at C-3′-13-O-glucose) produces rebaudioside D.

Any suitable technique known in the art for isolating and/or purifying compounds, such as rebaudiosides from plants, such as Stevia, may be used. For example, rebaudiosides can be isolated and/or purified from Stevia plant material utilizing one or more of the techniques described in U.S. Pat. Nos. 3,723,410; 4,082,858; 4,361,697; 4,599,403; 5,112,610; 5,962,678; 8,299,224; 8,414,951; U.S. Patent Application Publication Nos. 2006/0083838; 2006/0134292; 2007/0082103; 2008/0300402; and Chaturvedula, VSP and Prakash, I, Eur. Chem. Bull. 2013, 2(5), 298-302. Such techniques are incorporated herein by reference. Alternatively, the compounds can be recombinantly produced or chemically synthesized using methods well known to those of skill in the art.

In some embodiments, glycosides from leaves, such as rebaudiosides, can be extracted using either water or organic solvent extraction. Supercritical fluid extraction and steam distillation can also be used. In other embodiments, rebaudiosides can be recovered from Stevia plants using membrane technology. In some embodiments, production of an extract typically includes extraction of plant material with water or an water-organic solvent mixture, precipitation of high molecular weight substances, deionization and decolorization, purification on specific macroporous polymeric adsorbents, concentration, and drying.

In other embodiments, extracts of Stevia leaves may be purified to concentrate a selected component of the Stevia extract. For example, column chromatography may be used to isolate rebaudiosides from the other diterpene glycosides. In some embodiments, following chromatographic separation, the produced rebaudioside may optionally be recrystallized at least once, or at least twice, or at least three times, to obtain a Stevia extract containing a desired level of purity of the rebaudioside.

In some embodiments, a Stevia extract used in the steviol glycoside formulations provided herein has a purity of about 50% to about 100% by weight, about 55% to about 100% by weight, about 60% to about 100% by weight, about 65% to about 100% by weight, about 70% to about 100% by weight, about 75% to about 100% by weight, about 80% to about 100% by weight, about 85% to about 100% by weight, about 86% to about 100% by weight, about 87% to about 100% by weight, about 88% to about 100% by weight, about 89% to about 100% by weight, about 90% to about 100% by weight, about 91% to about 100% by weight, about 92% to about 100% by weight, about 93% to about 100% by weight, about 94% to about 100% by weight, about 95% to about 100% by weight, about 96% to about 100% by weight, about 97% to about 100% by weight, about 98% to about 100% by weight, or about 99% to about 100% by weight.

Alternatively, a Stevia extract used in the steviol glycoside formulations provided herein has a purity of about 50% to about 100% by weight, about 50% to about 99% by weight, about 50% to about 98% by weight, about 50% to about 97% by weight, about 50% to about 96% by weight, about 50% to about 95% by weight, about 50% to about 94% by weight, about 50% to about 93% by weight, about 50% to about 92% by weight, about 50% to about 91% by weight, about 50% to about 90% by weight, about 50% to about 85% by weight, about 50% to about 80% by weight, about 50% to about 75% by weight, about 50% to about 70% by weight, about 50% to about 65% by weight, about 50% to about 60% by weight, or about 50% to about 55% by weight. For example, a Stevia extract used in a steviol glycoside formulation provided herein may have a purity of about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or about 100% by weight, including any range in between these values.

The purity of rebaudiosides, such as those extracted, isolated, and/or purified from Stevia plants, can be assayed using any suitable method known in the art. For example, chromatography, such as HPLC, may be used to test the purity of rebaudioside extracts.

In some embodiments, production of the steviol glycosides used herein can be accomplished through the utilization of microbial strains to produce various rebaudiosides in high yield and purity to allow commercial incorporation into food products (See, e.g., U.S. Pat. Nos. 9,988,414, 9,522,929, 10,010,099, 10,010,101, 10,081,826, 10,253,344 all of which, including the methods of production, are incorporated herein by reference).

In some embodiments, rebaudiosides may be produced by recombinantly expressing enzymes in a microbial system (e.g., a host cell) capable of producing steviol. In general, such enzymes include: an UDP-glycosyltransferase, a beta glycosidase, a rhamnosyltransferase, a copalyl diphosphate synthase (CPS), a kaurene synthase (KS) and a geranylgeranyl diphosphate to synthase (GGPPS) enzyme, and functional fragments or variants thereof. In some embodiments, this can occur in a microbial strain that expresses an endogenous isoprenoid synthesis pathway, such as the non-mevalonate (MEP) pathway or the mevalonic acid pathway (MVA). In some embodiments, the microbial system (e.g., a host cell) further expresses additional enzymes (e.g., sucrose synthase or SUS).

In some embodiments, the host cell is selected from the group consisting of Escherichia; Salmonella; Bacillus; Acinetobacter; Streptomyces; Corynebacterium; Methylosinus; Methylomonas; Rhodococcus; Pseudomonas; Rhodobacter; Synechocystis; Saccharomyces; Zygosaccharomyces; Kluyveromyces; Candida; Hansenula; Debaryomyces; Mucor; Pichia; Torulopsis; Aspergillus; Arthrobotlys; Brevibacteria; Microbacterium; Arthrobacter; Citrobacter; Klebsiella; Pantoea; Corynebacterium; Clostridium (e.g., Clostridium acetobutylicum). In some embodiments, the host cell is a cell isolated from plants selected from the group consisting of soybean; rapeseed; sunflower; cotton; corn; tobacco; alfalfa; wheat; barley; oats; sorghum; rice; broccoli; cauliflower; cabbage; parsnips; melons; carrots; celery; parsley; tomatoes; potatoes; strawberries; peanuts; grapes; grass seed crops; sugar beets; sugar cane; beans; peas; rye; flax; hardwood trees; softwood trees; forage grasses; Arabidopsis thaliana; rice (Oryza sativa); Hordeum yulgare; switchgrass (Panicum vigratum); Brachypodium spp.; Brassica spp.; and Crambe abyssinica. In some embodiments, the cell is a bacterial cell, such as E. coli, or a yeast cell, such as a Saccharomyces cell, Pichia cell, or a Yarrowia cell. In some embodiments, the cell is an algal cell or a plant cell.

In some embodiments, rebaudiosides of the formulations provided herein are produced in a reaction mixture including a start compound (e.g., any natural or synthetic compound capable of being converted into a steviol glycoside compound in a reaction catalyzed by one or more enzymes); a substrate selected from the group consisting of sucrose, uridine diphosphate (UDP) and uridine diphosphate-glucose (UDP-glucose); and the one or more enzymes, such as a UDP-glycosyltransferase.

Suitable UDP-glycosyltransferases for producing rebaudiosides in either a microbial system or an in vitro reaction mixture include any UGT known in the art as capable of catalyzing one or more reactions in the biosynthesis of steviol glycoside compounds, such as, without limitation, EUGT11 (GenBank Accession No. AC133334), HV1 (GenBank Accession No. BAJ98242.1), UGT76G1 (Genbank Accession No. AAR06912.1), UGT85C2 (GenBank Accession No. AAR06916.1), UGT74G1 (GenBank Accession No. AAR06920.1), or the functional homologs, fragments, or variants thereof.

In some embodiments, the UDP-glycosyltransferase used in any one of the methods described herein is UGT76G1, or any functional fragments or variants thereof. Uridine diphospho glycosyltransferase (UGT76G1) is a UGT with a 1,3-13-O-glucose glycosylation activity that can produce related glycoside (rebaudioside A and D). UGT76G1 also has 1,3-19-O-glucose glycosylation activity that can produce rebaudioside G from rubusoside, and rebaudioside M from rebaudioside D. Amino acid sequences of UGT76G1 and variants (e.g., UGT76G1 CP1, CP1, and L200A mutants) are provided in Table 2.

In some embodiments, the UDP-glycotransferase used in any one of the methods described herein is EUGT11, or any functional fragments or variants thereof. EUGT11 is a UGT having 1,2-19-O-glucose and 1,2-13-O-glucose glycosylation activity. EUGT11 is known to catalyze the production of stevioside to rebaudioside E and rebaudioside A to rebaudioside D. EUGT11 also has 1,2-19-O-glucose glycosylation activity. Amino acid sequences of EUGT11 and variants (e.g., EUGT11 CP1 mutant) are provided in Table 2.

In some embodiments, the UDP-glycotransferase used in any one of the methods described herein is HV1, or any functional fragments or variants thereof. HV1 is a UGT with a 1,2-19-O-glucose glycosylation activity that can produce related steviol glycosides (rebaudioside E, D and Z). HV1 also can convert Reb KA to Reb E. Amino acid sequences of HV1 and variants are provided in Table 2.

In some embodiments, the UGT used for producing the rebaudiosides as described herein comprises an amino acid sequence that is at least 70% at least 80%, at least 85%, at least 90%, at least 91%, at least 92%, at least 93%, at least 94%, at least 95%, at least 96%, at least 97%, at least 98%, at least 99% and even 100% identical to the amino acid sequence set forth in any one of SEQ ID NOs:1-7. In some embodiments, the UGT used for producing the rebaudiosides as described herein comprises an amino acid sequence that is at least 80% identical to the amino acid sequence set forth in any one of SEQ ID NOs:1-7. In some embodiments, the UGT used for producing the rebaudiosides as described herein comprises an amino acid sequence that is at least 85%, at least 90%, at least 91%, at least 92%, at least 93%, at least 94%, at least 95%, at least 96%, at least 97%, at least 98%, or at least 99% to the amino acid sequence set forth in any one of SEQ ID NOs: 1-7. In some embodiments, the UGT used for producing the rebaudiosides as described herein comprises the amino acid sequence of any one of SEQ ID NOs: 1-7.

In some embodiments, the reaction mixture further comprises additional enzymes (e.g., sucrose synthase or SUS) to improve the efficiency or modify the outcome of the overall biosynthesis of steviol glycoside compounds. For example, the additional enzyme may regenerate the UDP-glucose needed for the glycosylation reaction by converting the UDP produced from the glycosylation reaction back to UDP-glucose (using, for example, sucrose as a donor of the glucose residue), thus improving the efficiency of the glycosylation reaction.

Suitable sucrose synthase domains can be for example, an Arabidopsis sucrose synthase 1; an Arabidopsis sucrose synthase 3 and a Vigna radiate sucrose synthase. A particularly suitable sucrose synthase domain can be, for example, Arabidopsis sucrose synthase 1. A particularly suitable Arabidopsis sucrose synthase 1 is Arabidopsis thaliana sucrose synthase 1 (AtSUS1). A particularly suitable sucrose synthase 1 domain can be, for example, a sucrose synthase 1 having the amino acid sequence of SEQ ID NO: 8.

In some embodiments, the SUS used for producing the rebaudiosides as described herein comprises an amino acid sequence that is at least 70% at least 80%, at least 85%, at least 90%, at least 91%, at least 92%, at least 93%, at least 94%, at least 95%, at least 96%, at least 97%, at least 98%, at least 99% and even 100% identical to the amino acid sequence set forth in SEQ ID NO: 8. In some embodiments, the SUS used for producing the rebaudiosides as described herein comprises an amino acid sequence that is at least 80% identical to the amino acid sequence set forth in SEQ ID NO: 8. In some embodiments, the SUS used for producing the rebaudiosides as described herein comprises an amino acid sequence that is at least 85%, at least 90%, at least 91%, at least 92%, at least 93%, at least 94%, at least 95%, at least 96%, at least 97%, at least 98%, or at least 99% to the amino acid sequence set forth in SEQ ID NO: 8. In some embodiments, the SUS used for producing the rebaudiosides as described herein comprises the amino acid sequence of SEQ ID NO: 8.

Sucrose synthase catalyzes the chemical reaction between UDP-glucose and D-fructose to produce UDP and sucrose. Sucrose synthase is a glycosyltransferase. The systematic name of this enzyme class is UDP-glucose:D-fructose 2-alpha-D -glucosyltransferase. Other names in common use include UDP glucose-fructose glucosyltransferase, sucrose synthetase, sucrose-UDP glucosyltransferase, sucrose-uridine diphosphate glucosyltransferase, and uridine diphosphoglucose-fructose glucosyltransferase. Addition of the sucrose synthase to the reaction mixture that includes a uridine diphospho glycosyltransferase creates a “UGT-SUS coupling system”. In the UGT-SUS coupling system, UDP-glucose can be regenerated from UDP and sucrose, which allows for omitting the addition of extra UDP-glucose to the reaction mixture or using UDP in the reaction mixture. Suitable sucrose synthase for use in the methods described herein include Arabidopsis sucrose synthase I, an Arabidopsis sucrose synthase 3 and a Vigna radiate sucrose synthase. In some embodiments of any one of the methods or compositions provided herein, the sucrose synthase or sucrose synthase domain is an Arabidopsis thaliana sucrose synthase I. In some embodiments, the UDP-glycotransferase used in any one of the methods described herein is a UGT-sucrose synthase fusion enzyme. In some embodiments, the UDP-glycotransferase used in any one of the methods described herein is a UGT76G1-sucrose synthase fusion enzyme. In some embodiments, the UDP-glycotransferase used in any one of the methods described herein is a EUGT11-sucrose synthase fusion enzyme. In some embodiments, the UDP-glycotransferase used in any one of the methods described herein is a HV1-sucrose synthase fusion enzyme. Amino acid sequences of examples of UGT-SUS fusion enzymes are provided in Table 2.

In some embodiments, the UGT-SUS fusion enzyme used for producing the rebaudiosides as described herein comprises an amino acid sequence that is at least 70% at least 80%, at least 85%, at least 90%, at least 91%, at least 92%, at least 93%, at least 94%, at least 95%, at least 96%, at least 97%, at least 98%, at least 99% and even 100% identical to the amino acid sequence set forth in any one of SEQ ID NOs: 9-11. In some embodiments, the UGT-SUS fusion enzyme used for producing the rebaudiosides as described herein comprises an amino acid sequence that is at least 80% identical to the amino acid sequence set forth in any one of SEQ ID NOs: 9-11. In some embodiments, the UGT-SUS fusion enzyme used for producing the rebaudiosides as described herein comprises an amino acid sequence that is at least 85%, at least 90%, at least 91%, at least 92%, at least 93%, at least 94%, at least 95%, at least 96%, at least 97%, at least 98%, or at least 99% to the amino acid sequence set forth in any one of SEQ ID NOs: 9-11. In some embodiments, the UGT-SUS fusion enzyme used for producing the rebaudiosides as described herein comprises the amino acid sequence of any one of SEQ ID NOs: 9-11.

In some embodiments, the rebaudiosides are produced in a reaction mixture including a start compound (e.g., any natural or synthetic compound capable of being converted into a steviol glycoside compound in a reaction catalyzed by one or more UDP-glucosyltransferases) and a beta glucosidase. Examples of beta glucosidases for use in this method include, without limitation, beta glucosidase 1 from Pichia pastoris, beta glucosidase 2 from Pichia pastoris, beta glucosidase 3 from Pichia pastoris, beta glucosidase 4 from Pichia pastoris, or any functional variants thereof.

In some embodiments, the beta glucosidase used for producing the rebaudiosides as described herein comprises an amino acid sequence that is at least 70% at least 80%, at least 85%, at least 90%, at least 91%, at least 92%, at least 93%, at least 94%, at least 95%, at least 96%, at least 97%, at least 98%, at least 99% and even 100% identical to the amino acid sequence set forth in any one of SEQ ID NOs: 12-15. In some embodiments, the beta glucosidase used for producing the rebaudiosides as described herein comprises an amino acid sequence that is at least 80% identical to the amino acid sequence set forth in any one of SEQ ID NOs: 12-15. In some embodiments, the beta glucosidase used for producing the rebaudiosides as described herein comprises an amino acid sequence that is at least 85%, at least 90%, at least 91%, at least 92%, at least 93%, at least 94%, at least 95%, at least 96%, at least 97%, at least 98%, or at least 99% to the amino acid sequence set forth in any one of SEQ ID NOs: 12-15. In some embodiments, the beta glucosidase used for producing the rebaudiosides as described herein comprises the amino acid sequence of any one of SEQ ID NOs: 12-15.

Nucleic acid sequences encoding any one of the enzymes described herein are also provided in Table 2. As known by those skilled in the art, the nucleic acid sequence encoding enzymes can be codon optimized for expression in a suitable host organism such as, for example, bacteria and yeast.

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by Greene Publishing and Wiley-Interscience, 1987; (the entirety of each of which is hereby incorporated herein by reference).

TABLE 2
Examples of enzymes used for
synthesizing rebaudiosides

| Name | Sequences |
|---|---|
| UGT76G1 | MENKTETTVRRRRRIILFPVPFQGHINPIL |
| WT Amino | QLANVLYSKGFSITIFHTNFNKPKTSNYPH |
| Acid | FTFRFILDNDPQDERISNLPTHGPLAGMRI |
| | PIINEHGADELRRELELLMLASEEDEEVSC |
| | LITDALWYFAQSVADSLNLRRLVLMTSSLF |
| | NFHAHVSLPQFDELGYLDPDDKTRLEEQAS |
| | GFPMLKVKDIKSAYSNWQILKEILGKMIKQ |
| | TKASSGVIWNSFKELEESELETVIREIPAP |
| | SFLIPLPKHLTASSSSLLDHDRTVFQWLDQ |
| | QPPSSVLYVSFGSTSEVDEKDFLEIARGLV |
| | DSKQSFLWVVRPGFVKGSTWVEPLPDGFLG |
| | ERGRIVKWVPQQEVLAHGAIGAFWTHSGWN |
| | STLESVCEGVPMIFSDFGLDQPLNARYMSD |
| | VLKVGVYLENGWERGEIANAIRRVMVDEEG |
| | EYIRQNARVLKQKADVSLMKGGSSYESLES |
| | LVSYISSL |
| | (SEQ ID NO: 1) |
| UGT76G1 | MENKTETTVRRRRRIILFPVPFQGHINPIL |
| L200A | QLANVLYSKGFSITIFHTNFNKPKTSNYPH |
| Amino | FTFRFILDNDPQDERISNLPTHGPLAGMRI |
| Acid | PIINEHGADELRRELELLMLASEEDEEVSC |
| | LITDALWYFAQSVADSLNLRRLVLMTSSLF |
| | NFHAHVSLPQFDELGYLDPDDKTRLEEQAS |
| | GFPMLKVKDIKSAYSNWQIAKEILGKMIKQ |
| | TKASSGVIWNSFKELEESELETVIREIPAP |
| | SFLIPLPKHLTASSSSLLDHDRTVFQWLDQ |
| | QPPSSVLYVSFGSTSEVDEKDFLEIARGLV |
| | DSKQSFLWVVRPGFVKGSTWVEPLPDGFLG |
| | ERGRIVKWVPQQEVLAHGAIGAFWTHSGWN |
| | STLESVCEGVPMIFSDFGLDQPLNARYMSD |
| | VLKVGVYLENGWERGEIANAIRRVMVDEEG |
| | EYIRQNARVLKQKADVSLMKGGSSYESLES |
| | LVSYISSL |
| | (SEQ ID NO: 2) |
| UGT76G1 | MNWQILKEILGKMIKQTKASSGVIWNSFKE |
| CP1 Amino | LEESELETVIREIPAPSFLIPLPKHLTASS |
| Acid | SSLLDHDRTVFQWLDQQPPSSVLYVSFGST |
| | SEVDEKDFLEIARGLVDSKQSFLWVVRPGF |
| | VKGSTWVEPLPDGFLGERGRIVKWVPQQEV |
| | LAHGAIGAFWTHSGWNSTLESVCEGVPMIF |
| | SDFGLDQPLNARYMSDVLKVGVYLENGWER |
| | GEIANAIRRVMVDEEGEYIRQNARVLKQKA |
| | DVSLMKGGSSYESLESLVSYISSLENKTET |
| | TVRRRRRIILFPVPFQGHINPILQLANVLY |
| | SKGFSITIFHTNFNKPKTSNYPHFTFRFIL |
| | DNDPQDERISNLPTHGPLAGMRIPIINEHG |
| | ADELRRELELLMLASEEDEEVSCLITDALW |
| | YFAQSVADSLNLRRLVLMTSSLFNFHAHVS |
| | LPQFDELGYLDPDDKTRLEEQASGFPMLKV |
| | KDIKSAYS |
| | (SEQ ID NO: 3) |
| UGT76G1 | MNWQILKEILGKMIKQTKASSGVIWNSFKE |
| CP2 Amino | LEESELETVIREIPAPSFLIPLPKHLTASS |
| Acid | SSLLDHDRTVFQWLDQQPPSSVLYVSFGST |
| | SEVDEKDFLEIARGLVDSKQSFLWVVRPGF |
| | VKGSTWVEPLPDGFLGERGRIVKWVPQQEV |
| | LAHGAIGAFWTHSGWNSTLESVCEGVPMIF |
| | SDFGLDQPLNARYMSDVLKVGVYLENGWER |
| | GEIANAIRRVMVDEEGEYIRQNARVLKQKA |
| | DVSLMKGGSSYESLESLVSYISSLYKDDSG |
| | YSSSYAAAAGMENKTETTVRRRRRIILFPV |
| | PFQGHINPILQLANVLYSKGFSITIFHTNF |
| | NKPKTSNYPHFTFRFILDNDPQDERISNLP |
| | THGPLAGMRIPIINEHGADELRRELELLML |
| | ASEEDEEVSCLITDALWYFAQSVADSLNLR |
| | RLVLMTSSLFNFHAHVSLPQFDELGYLDPD |
| | DKTRLEEQASGFPMLKVKDIKSAYS |
| | (SEQ ID NO: 4) |
| EUGT11 | MDSGYSSSYAAAAGMHVVICPWLAFGHLLP |
| WT | CLDLAQRLASRGHRVSFVSTPRNISRLPPV |
| Amino | RPALAPLVAFVALPLPRVEGLPDGAESTND |
| Acid | VPHDRPDMVELHRRAFDGLAAPFSEFLGTA |
| | CADWVIVDVFHHWAAAAALEHKVPCAMMLL |
| | GSAHMIASIADRRLERAETESPAAAGQGRP |
| | AAAPTFEVARMKLIRTKGSSGMSLAERFSL |
| | TLSRSSLVVGRSCVEFEPETVPLLSTLRGK |
| | PITFLGLMPPLHEGRREDGEDATVRWLDAQ |
| | PAKSVVYVALGSEVPLGVEKVHELALGLEL |
| | AGTRFLWALRKPTGVSDADLLPAGFEERTR |
| | GRGVVATRWVPQMSILAHAAVGAFLTHCGW |
| | NSTIEGLMFGHPLIMLPIFGDQGPNARLIE |
| | AKNAGLQVARNDGDGSFDREGVAAAIRAVA |
| | VEEESSKVFQAKAKKLQEIVADMACHERYI |
| | DGFIQQLRSYKD |
| | (SEQ ID NO: 5) |
| EUGT11 | MGSSGMSLAERFSLTLSRSSLVVGRSCVEF |
| CP1 | EPETVPLLSTLRGKPITFLGLMPPLHEGRR |
| Amino | EDGEDATVRWLDAQPAKSVVYVALGSEVPL |
| Acid | GVEKVHELALGLELAGTRFLWALRKPTGVS |
| | DADLLPAGFEERTRGRGVVATRWVPQMSIL |
| | AHAAVGAFLTHCGWNSTIEGLMFGHPLIML |
| | PIFGDQGPNARLIEAKNAGLQVARNDGDGS |
| | FDREGVAAAIRAVAVEEESSKVFQAKAKKL |
| | QEIVADMACHERYIDGFIQQLRSYKDDSGY |
| | SSSYAAAAGMHVVICPWLAFGHLLPCLDLA |
| | QRLASRGHRVSFVSTPRNISRLPPVRPALA |
| | PLVAFVALPLPRVEGLPDGAESTNDVPHDR |
| | PDMVELHRRAFDGLAAPFSEFLGTACADWV |
| | IVDVFHHWAAAAALEHKVPCAMMLLGSAHM |
| | IASIADRRLERAETESPAAAGQGRPAAAPT |
| | FEVARMKLIRTK |
| | (SEQ ID NO: 6) |
| HV1 | MDGNSSSSPLHVVICPWLALGHLLPCLDIA |
| glycosyl | ERLASRGHRVSFVSTPRNIARLPPLRPAVA |
| transferase | PLVDFVALPLPHVDGLPEGAESTNDVPYDK |
| amino acid | FELHRKAFDGLAAPFSEFLRAACAEGAGSR |
| | PDWLIVDTFHHWAAAAAVENKVPCVMLLLG |
| | AATVIAGFARGVSEHAAAAVGKERPAAEAP |
| | SFETERRKLMTTQNASGMTVAERYFLTLMR |
| | SDLVAIRSCAEWEPESVAALTTLAGKPVVP |
| | LGLLPPSPEGGRGVSKEDAAVRWLDAQPAK |
| | SVVYVALGSEVPLRAEQVHELALGLELSGA |
| | RFLWALRKPTDAPDAAVLPPGFEERTRGRG |
| | LVVTGWVPQIGVLAHGAVAAFLTHCGWNST |
| | IEGLLFGHPLIMLPISSDQGPNARLMEGRK |
| | VGMQVPRDESDGSFRREDVAATVRAVAVEE |
| | DGRRVFTANAKKMQEIVADGACHERCIDGF |
| | IQQLRSYKA |
| | (SEQ ID NO: 7) |
| SUS1 WT | MANAERMITRVHSQRERLNETLVSERNEVL |
| from | ALLSRVEAKGKGILQQNQIIAEFEALPEQT |
| Arabidopsis | RKKLEGGPFFDLLKSTQEAIVLPPWVALAV |
| thaliana, | RPRPGVWEYLRVNLHALVVEELQPAEFLHF |
| Amino | KEELVDGVKNGNFTLELDFEPFNASIPRPT |
| Acid | LHKYIGNGVDFLNRHLSAKLFHDKESLLPL |
| | LKFLRLHSHQGKNLMLSEKIQNLNTLQHTL |
| | RKAEEYLAELKSETLYEEFEAKFEEIGLER |
| | GWGDNAERVLDMIRLLLDLLEAPDPCTLET |
| | FLGRVPMVFNVVILSPHGYFAQDNVLGYPD |
| | TGGQVVYILDQVRALEIEMLQRIKQQGLNI |
| | KPRILILTRLLPDAVGTTCGERLERVYDSE |
| | YCDILRVPFRTEKGIVRKWISRFEVWPYLE |
| | TYTEDAAVELSKELNGKPDLIIGNYSDGNL |
| | VASLLAHKLGVTQCTIAHALEKTKYPDSDI |
| | YWKKLDDKYHFSCQFTADIFAMNHTDFIIT |
| | STFQELAGSKETVGQYESHTAFTLPGLYRV |
| | VHGIDVFDPKFNIVSPGADMSIYFPYTEEK |
| | RRLTKFHSEIEELLYSDVENKEHLCVLKDK |
| | KKPILFTMARLDRVKNLSGLVEWYGKNTRL |
| | RELANLVVVGGDRRKESKDNEEKAEMKKMY |
| | DLIEEYKLNGQFRWISSQMDRVRNGELYRY |
| | ICDTKGAFVQPALYEAFGLTVVEAMTCGLP |
| | TFATCKGGPAEIIVHGKSGFHIDPYHGDQA |
| | ADTLADFFTKCKEDPSHWDEISKGGLQRIE |
| | EKYTWQIYSQRLLTLTGVYGFWKHVSNLDR |
| | LEARRYLEMFYALKYRPLAQAVPLAQDD |
| | (SEQ ID NO: 8) |
| UGT76G1- | MENKTETTVRRRRRIILFPVPFQGHINPIL |
| AtSUS1 | QLANVLYSKGFSITIFHTNFNKPKTSNYPH |
| fusion | FTFRFILDNDPQDERISNLPTHGPLAGMRI |
| enzyme, | PIINEHGADELRRELELLMLASEEDEEVSC |
| amino acid | LITDALWYFAQSVADSLNLRRLVLMTSSLF |
| | NFHAHVSLPQFDELGYLDPDDKTRLEEQAS |
| | GFPMLKVKDIKSAYSNWQILKEILGKMIKQ |
| | TKASSGVIWNSFKELEESELETVIREIPAP |
| | SFLIPLPKHLTASSSSLLDHDRTVFQWLDQ |
| | QPPSSVLYVSFGSTSEVDEKDFLEIARGLV |
| | DSKQSFLWVVRPGFVKGSTWVEPLPDGFLG |
| | ERGRIVKWVPQQEVLAHGAIGAFWTHSGWN |
| | STLESVCEGVPMIFSDFGLDQPLNARYMSD |
| | VLKVGVYLENGWERGEIANAIRRVMVDEEG |
| | EYIRQNARVLKQKADVSLMKGGSSYESLES |
| | LVSYISSLGSGANAERMITRVHSQRERLNE |
| | TLVSERNEVLALLSRVEAKGKGILQQNQII |
| | AEFEALPEQTRKKLEGGPFFDLLKSTQEAI |
| | VLPPWVALAVRPRPGVWEYLRVNLHALVVE |
| | ELQPAEFLHFKEELVDGVKNGNFTLELDFE |
| | PFNASIPRPTLHKYIGNGVDFLNRHLSAKL |
| | FHDKESLLPLLKFLRLHSHQGKNLMLSEKI |
| | QNLNTLQHTLRKAEEYLAELKSETLYEEFE |
| | AKFEEIGLERGWGDNAERVLDMIRLLLDLL |
| | EAPDPCTLETFLGRVPMVFNVVILSPHGYF |
| | AQDNVLGYPDTGGQVVYILDQVRALEIEML |
| | QRIKQQGLNIKPRILILTRLLPDAVGTTCG |
| | ERLERVYDSEYCDILRVPFRTEKGIVRKWI |
| | SRFEVWPYLETYTEDAAVELSKELNGKPDL |
| | IIGNYSDGNLVASLLAHKLGVTQCTIAHAL |
| | EKTKYPDSDIYWKKLDDKYHFSCQFTADIF |
| | AMNHTDFIITSTFQEIAGSKETVGQYESHT |
| | AFTLPGLYRVVHGIDVFDPKFNIVSPGADM |
| | SIYFPYTEEKRRLTKFHSEIEELLYSDVEN |
| | KEHLCVLKDKKKPILFTMARLDRVKNLSGL |
| | VEWYGKNTRLRELANLVVVGGDRRKESKDN |
| | EEKAEMKKMYDLIEEYKLNGQFRWISSQMD |
| | RVRNGELYRYICDTKGAFVQPALYEAFGLT |
| | VVEAMTCGLPTFATCKGGPAEIIVHGKSGF |
| | HIDPYHGDQAADTLADFFTKCKEDPSHWDE |
| | ISKGGLQRIEEKYTWQIYSQRLLTLTGVYG |
| | FWKHVSNLDRLEARRYLEMFYALKYRPLAQ |
| | AVPLAQDDWT |
| | (SEQ ID NO: 9) |
| EUGT11- | MDSGYSSSYAAAAGMHVVICPWLAFGHLLP |
| AtSUS1 | CLDLAQRLASRGHRVSFVSTPRNISRLPPV |
| fusion | RPALAPLVAFVALPLPRVEGLPDGAESTND |
| enzyme, | VPHDRPDMVELHRRAFDGLAAPFSEFLGTA |
| amino acid | CADWVIVDVFHHWAAAAALEHKVPCAMMLL |
| | GSAHMIASIADRRLERAETESPAAAGQGRP |
| | AAAPTFEVARMKLIRTKGSSGMSLAERFSL |
| | TLSRSSLVVGRSCVEFEPETVPLLSTLRGK |
| | PITFLGLMPPLHEGRREDGEDATVRWLDAQ |
| | PAKSVVYVALGSEVPLGVEKVHELALGLEL |
| | AGTRFLWALRKPTGVSDADLLPAGFEERTR |
| | GRGVVATRWVPQMSILAHAAVGAFLTHCGW |
| | NSTIEGLMFGHPLIMLPIFGDQGPNARLIE |
| | AKNAGLQVARNDGDGSFDREGVAAAIRAVA |
| | VEEESSKVFQAKAKKLQEIVADMACHERYI |
| | DGFIQQLRSYKDGSGANAERMITRVHSQRE |
| | RLNETLVSERNEVLALLSRVEAKGKGILQQ |
| | NQIIAEFEALPEQTRKKLEGGPFFDLLKST |
| | QEAIVLPPWVALAVRPRPGVWEYLRVNLHA |
| | LVVEELQPAEFLHFKEELVDGVKNGNFTLE |
| | LDFEPFNASIPRPTLHKYIGNGVDFLNRHL |
| | SAKLFHDKESLLPLLKFLRLHSHQGKNLML |
| | SEKIQNLNTLQHTLRKAEEYLAELKSETLY |
| | EEFEAKFEEIGLERGWGDNAERVLDMIRLL |
| | LDLLEAPDPCTLETFLGRVPMVFNVVILSP |
| | HGYFAQDNVLGYPDTGGQVVYILDQVRALE |
| | IEMLQRIKQQGLNIKPRILILTRLLPDAVG |
| | TTCGERLERVYDSEYCDILRVPFRTEKGIV |
| | RKWISRFEVWPYLETYTEDAAVELSKELNG |
| | KPDLIIGNYSDGNLVASLLAHKLGVTQCTI |
| | AHALEKTKYPDSDIYWKKLDDKYHFSCQFT |
| | ADIFAMNHTDFIITSTFQEIAGSKETVGQY |
| | ESHTAFTLPGLYRVVHGIDVFDPKFNIVSP |
| | GADMSIYFPYTEEKRRLTKFHSEIEELLYS |
| | DVENKEHLCVLKDKKKPILFTMARLDRVKN |
| | LSGLVEWYGKNTRLRELANLVVVGGDRRKE |
| | SKDNEEKAEMKKMYDLIEEYKLNGQFRWIS |
| | SQMDRVRNGELYRYICDTKGAFVQPALYEA |
| | FGLTVVEAMTCGLPTFATCKGGPAEIIVHG |
| | KSGFHIDPYHGDQAADTLADFFTKCKEDPS |
| | HWDEISKGGLQRIEEKYTWQIYSQRLLTLT |
| | GVYGFWKHVSNLDRLEARRYLEMFYALKYR |
| | PLAQAVPLAQDD |
| | (SEQ ID NO: 10) |
| HV1- | MDGNSSSSPLHVVICPWLALGHLLPCLDIA |
| AtSUS1 | ERLASRGHRVSFVSTPRNIARLPPLRPAVA |
| fusion | PLVDFVALPLPHVDGLPEGAESTNDVPYDK |
| enzyme, | FELHRKAFDGLAAPFSEFLRAACAEGAGSR |
| amino acid | PDWLIVDTFHHWAAAAAVENKVPCVMLLLG |
| | AATVIAGFARGVSEHAAAAVGKERPAAEAP |
| | SFETERRKLMTTQNASGMTVAERYFLTLMR |
| | SDLVAIRSCAEWEPESVAALTTLAGKPVVP |
| | LGLLPPSPEGGRGVSKEDAAVRWLDAQPAK |
| | SVVYVALGSEVPLRAEQVHELALGLELSGA |
| | RFLWALRKPTDAPDAAVLPPGFEERTRGRG |
| | LVVTGWVPQIGVLAHGAVAAFLTHCGWNST |
| | IEGLLFGHPLIMLPISSDQGPNARLMEGRK |
| | VGMQVPRDESDGSFRREDVAATVRAVAVEE |
| | DGRRVFTANAKKMQEIVADGACHERCIDGF |
| | IQQLRSYKAGSGANAERMITRVHSQRERLN |
| | ETLVSERNEVLALLSRVEAKGKGILQQNQI |
| | IAEFEALPEQTRKKLEGGPFFDLLKSTQEA |
| | IVLPPWVALAVRPRPGVWEYLRVNLHALVV |
| | EELQPAEFLHFKEELVDGVKNGNFTLELDF |
| | EPFNASIPRPTLHKYIGNGVDFLNRHLSAK |
| | LFHDKESLLPLLKFLRLHSHQGKNLMLSEK |
| | IQNLNTLQHTLRKAEEYLAELKSETLYEEF |
| | EAKFEEIGLERGWGDNAERVLDMIRLLLDL |
| | LEAPDPCTLETFLGRVPMVFNVVILSPHGY |
| | FAQDNVLGYPDTGGQVVYILDQVRALEIEM |
| | LQRIKQQGLNIKPRILILTRLLPDAVGTTC |
| | GERLERVYDSEYCDILRVPFRTEKGIVRKW |
| | ISRFEVWPYLETYTEDAAVELSKELNGKPD |
| | LIIGNYSDGNLVASLLAHKLGVTQCTIAHA |
| | LEKTKYPDSDIYWKKLDDKYHFSCQFTADI |
| | FAMNHTDFIITSTFQEIAGSKETVGQYESH |
| | TAFTLPGLYRVVHGIDVFDPKFNIVSPGAD |
| | MSIYFPYTEEKRRLTKFHSEIEELLYSDVE |
| | NKEHLCVLKDKKKPILFTMARLDRVKNLSG |
| | LVEWYGKNTRLRELANLVVVGGDRRKESKD |
| | NEEKAEMKKMYDLIEEYKLNGQFRWISSQM |
| | DRVRNGELYRYICDTKGAFVQPALYEAFGL |
| | TVVEAMTCGLPTFATCKGGPAEIIVHGKSG |
| | FHIDPYHGDQAADTLADFFTKCKEDPSHWD |
| | EISKGGLQRIEEKYTWQIYSQRLLTLTGVY |
| | GFWKHVSNLDRLEARRYLEMFYALKYRPLA |
| | QAVPLAQDD |
| | (SEQ ID NO: 11) |
| Beta- | MTQLDVESLIQELTLNEKVQLLSGSDFWHT |
| glucosidase | TPVRRLGIPKMRLSDGPNGVRGTKFFNGVP |
| 1 from | TACFPCGTGLGATFDKELLKEAGSLMADEA |
| Pichia | KAKAASVVLGPTANIARGPNGGRGFESFGE |
| pastoris, | DPVVNGLSSAAMINGLQGKYLAATMKHYVC |
| amino acid | NDLEMDRNCIDAQVSHRALREVYLLPFQIA |
| | VRDANPRAIMTAYNKANGEHVSQSKFLLDE |
| | VLRKEWGWDGLLMSDWFGVYDAKSSITNGL |
| | DLEMPGPPQCRVHSATDHAINSGEIHINDV |
| | DERVRSLLSLINYCHQSGVTEEDPETSDNN |
| | TPETIEKLRKISRESIVLLKDDDRNRSILP |
| | LKKSDKIAVIGNNAKQAAYCGGGSASVLSY |
| | HTTTPFDSIKSRLEDSNTPAYTIGADAYKN |
| | LPPLGPQMTDSDGKPGFDAKFFVGSPTSKD |
| | RKLIDHFQLTNSQVFLVDYYNEQIPENKEF |
| | YVDVEGQFIPEEDGTYNFGLTVFGTGRLFV |
| | DDKLVSDSSQNQTPGDSFFGLAAQEVIGSI |
| | HLVKGKAYKIKVLYGSSVTRTYEIAASVAF |
| | EGGAFTFGAAKQRNEDEELARAVEIAKAND |
| | KVVLCIGLNQDFESEGFDRPDIKIPGATNK |
| | MVSAVLKANPNTVIVNQTGTPVEMPWASDA |
| | PVILQAWFGGSEAGTAIADVLFGDYNPSGK |
| | LTVTFPLRFEDNPAYLNFQSNKQACWYGED |
| | VYVGYRYYETIDRPVLFPFGHGLSFTEFDF |
| | TDMFVRLEEENLEVEVVVRNTGKYDGAEVV |
| | QLYVAPVSPSLKRPIKELKEYAKIFLASGE |
| | AKTVHLSVPIKYATSFFDEYQKKWCSEKGE |
| | YTILLGSSSADIKVSQSITLEKTTFWKGL |
| | (SEQ ID NO: 12) |
| Beta- | MKSQLIFMALASLVASAPLEHQQQHHKHEK |
| glucosidase | RAVVTQTVTVAAGQTAAAGSAQAVVTSSAA |
| 2 from | PASVASSAAASASSSSSSYTSGASGDLSSF |
| Pichia | KDGTIKCSEFPSGDGVVSVSWLGFGGWSSI |
| pastoris, | MNLQGGTSESCENGYYCSYACEAGYSKTQW |
| amino acid | PSNQPSDGRSVGGLLCKDGLLYRSNTAFDT |
| | LCVPGKGTASVENNVSKGISICRTDYPGSE |
| | NMCVPTWVDAGNSNTLTVVDEDNYYEWQGL |
| | KTSAQYYVNNAGVSVEDGCIWGDESSGVGN |
| | WAPLVLGAGSTGGLTYLSLIPNPNNKKAPN |
| | FNVKIVATDGSSINGDCKYENGIFVGSSTD |
| | GCTVTVTSGSAKLVFY |
| | (SEQ ID NO: 13) |
| Beta- | MQVKSIVNLLLACSLAVARPLEHAHHQHDK |
| glucosidase | RGVVVVTKTIVVDGSTVEATAAAQVQEHAE |
| 3 from | TFAESTPSAVVSSSSAPSSASSASAPASSG |
| Pichia | SFSAGTKGVTYSPYQAGGGCKTAEEVASDL |
| pastoris, | SQLTGYEIIRLYGVDCNQVENVFKAKAPGQ |
| amino acid | KLFLGIFFVDAIESGVSAIASAVKSYGSWD |
| | DVHTVSVGNELVNNGEATVSQIGQYVSTAK |
| | SALRSAGFTGPVLSVDTFIAVINNPGLCDF |
| | ADEYVAVNAHAFFDGGIAASGAGDWAAEQI |
| | QRVSSACGGKDVLIVESGWPSKGDTNGAAV |
| | PSKSNQQAAVQSLGQKIGSSCIAFNAFNDY |
| | WKADGPFNAEKYWGILDS |
| | (SEQ ID NO: 14) |
| Beta- | MLSTILNIFILLLFIQASLQAPIPVVTKYV |
| glucosidase | TEGIAVVTETNVRVVTKTIPIVQVLISDGA |
| 4 from | TYTHTLTTVSTAEENGNFQPITTTSIVNKE |
| Pichia | VVVPTSVTPNTQQTRPTQVDTTQNNADTPA |
| pastoris, | APTPSPTTSSNNGVFTTYSTTRSVVTSVVV |
| amino acid | VGPDGSPIENTGQTANPTTTAPTTSTTAAR |
| | TTSSTSTTPTASSTPGGNHPRSIVYSPYSD |
| | SSQCKDATTIETDLEFIASKGISAVRIYGN |
| | DCNYLTVVLPKCASLGLKVNQGFWIGPSGV |
| | DSIDDAVQEFIQAVNGNNGFNWDLFELITV |
| | GNEAISAGYVSASSLISKIKEVSSILSSAG |
| | YTGPITTAEPPNVYEDYGDLCSTDVMSIVG |
| | VNAHSYFNTLFAASDSGSFVKSQIEVVQKA |
| | CSRSDITIIETGYPSQGATNGKNVPSKENQ |
| | KTAIFSIFEVVGTDVTILSTYDDLWKDPGP |
| | YGIEQFFGAIDLFS |
| | (SEQ ID NO: 15) |
| UGT76G1 | ATGGAGAATAAGACAGAAACAACCGTAAGA |
| WT DNA | CGGAGGCGGAGGATTATCTTGTTCCCTGTA |
| | CCATTTCAGGGCCATATTAATCCGATCCTC |
| | CAATTAGCAAACGTCCTCTACTCCAAGGGA |
| | TTTTCAATAACAATCTTCCATACTAACTTT |
| | AACAAGCCTAAAACGAGTAATTATCCTCAC |
| | TTTACATTCAGGTTCATTCTAGACAACGAC |
| | CCTCAGGATGAGCGTATCTCAAATTTACCT |
| | ACGCATGGCCCCTTGGCAGGTATGCGAATA |
| | CCAATAATCAATGAGCATGGAGCCGATGAA |
| | CTCCGTCGCGAGTTAGAGCTTCTCATGCTC |
| | GCAAGTGAGGAAGACGAGGAAGTTTCGTGC |
| | CTAATAACTGATGCGCTTTGGTACTTCGCC |
| | CAATCAGTCGCAGACTCACTGAATCTACGC |
| | CGTTTGGTCCTTATGACAAGTTCATTATTC |
| | AACTTTCACGCACATGTATCACTGCCGCAA |
| | TTTGACGAGTTGGGTTACCTGGACCCGGAT |
| | GACAAAACGCGATTGGAGGAACAAGCGTCG |
| | GGCTTCCCCATGCTGAAAGTCAAAGATATT |
| | AAGAGCGCTTATAGTAATTGGCAAATTCTG |
| | AAAGAAATTCTCGGAAAAATGATAAAGCAA |
| | ACCAAAGCGTCCTCTGGAGTAATCTGGAAC |
| | TCCTTCAAGGAGTTAGAGGAATCTGAACTT |
| | GAAACGGTCATCAGAGAAATCCCCGCTCCC |
| | TCGTTCTTAATTCCACTACCCAAGCACCTT |
| | ACTGCAAGTAGCAGTTCCCTCCTAGATCAT |
| | GACCGAACCGTGTTTCAGTGGCTGGATCAG |
| | CAACCCCCGTCGTCAGTTCTATATGTAAGC |
| | TTTGGGAGTACTTCGGAAGTGGATGAAAAG |
| | GACTTCTTAGAGATTGCGCGAGGGCTCGTG |
| | GATAGCAAACAGAGCTTCCTGTGGGTAGTG |
| | AGACCGGGATTCGTTAAGGGCTCGACGTGG |
| | GTCGAGCCGTTGCCAGATGGTTTTCTAGGG |
| | GAGAGAGGGAGAATCGTGAAATGGGTTCCA |
| | CAGCAAGAGGTTTTGGCTCACGGAGCTATA |
| | GGGGCCTTTTGGACCCACTCTGGTTGGAAT |
| | TCTACTCTTGAAAGTGTCTGTGAAGGCGTT |
| | CCAATGATATTTTCTGATTTTGGGCTTGAC |
| | CAGCCTCTAAACGCTCGCTATATGTCTGAT |
| | GTGTTGAAGGTTGGCGTGTACCTGGAGAAT |
| | GGTTGGGAAAGGGGGGAAATTGCCAACGCC |
| | ATACGCCGGGTAATGGTGGACGAGGAAGGT |
| | GAGTACATACGTCAGAACGCTCGGGTTTTA |
| | AAACAAAAAGCGGACGTCAGCCTTATGAAG |
| | GGAGGTAGCTCCTATGAATCCCTAGAATCC |
| | TTGGTAAGCTATATATCTTCGTTATAA |
| | (SEQ ID NO: 16) |
| UGT76G1 | ATGGAGAATAAGACAGAAACAACCGTAAGA |
| L200A | CGGAGGCGGAGGATTATCTTGTTCCCTGTA |
| DNA | CCATTTCAGGGCCATATTAATCCGATCCTC |
| | CAATTAGCAAACGTCCTCTACTCCAAGGGA |
| | TTTTCAATAACAATCTTCCATACTAACTTT |
| | AACAAGCCTAAAACGAGTAATTATCCTCAC |
| | TTTACATTCAGGTTCATTCTAGACAACGAC |
| | CCTCAGGATGAGCGTATCTCAAATTTACCT |
| | ACGCATGGCCCCTTGGCAGGTATGCGAATA |
| | CCAATAATCAATGAGCATGGAGCCGATGAA |
| | CTCCGTCGCGAGTTAGAGCTTCTCATGCTC |
| | GCAAGTGAGGAAGACGAGGAAGTTTCGTGC |
| | CTAATAACTGATGCGCTTTGGTACTTCGCC |
| | CAATCAGTCGCAGACTCACTGAATCTACGC |
| | CGTTTGGTCCTTATGACAAGTTCATTATTC |
| | AACTTTCACGCACATGTATCACTGCCGCAA |
| | TTTGACGAGTTGGGTTACCTGGACCCGGAT |
| | GACAAAACGCGATTGGAGGAACAAGCGTCG |
| | GGCTTCCCCATGCTGAAAGTCAAAGATATT |
| | AAGAGCGCTTATAGTAATTGGCAAATTGCG |
| | AAAGAAATTCTCGGAAAAATGATAAAGCAA |
| | ACCAAAGCGTCCTCTGGAGTAATCTGGAAC |
| | TCCTTCAAGGAGTTAGAGGAATCTGAACTT |
| | GAAACGGTCATCAGAGAAATCCCCGCTCCC |
| | TCGTTCTTAATTCCACTACCCAAGCACCTT |
| | ACTGCAAGTAGCAGTTCCCTCCTAGATCAT |
| | GACCGAACCGTGTTTCAGTGGCTGGATCAG |
| | CAACCCCCGTCGTCAGTTCTATATGTAAGC |
| | TTTGGGAGTACTTCGGAAGTGGATGAAAAG |
| | GACTTCTTAGAGATTGCGCGAGGGCTCGTG |
| | GATAGCAAACAGAGCTTCCTGTGGGTAGTG |
| | AGACCGGGATTCGTTAAGGGCTCGACGTGG |
| | GTCGAGCCGTTGCCAGATGGTTTTCTAGGG |
| | GAGAGAGGGAGAATCGTGAAATGGGTTCCA |
| | CAGCAAGAGGTTTTGGCTCACGGAGCTATA |
| | GGGGCCTTTTGGACCCACTCTGGTTGGAAT |
| | TCTACTCTTGAAAGTGTCTGTGAAGGCGTT |
| | CCAATGATATTTTCTGATTTTGGGCTTGAC |
| | CAGCCTCTAAACGCTCGCTATATGTCTGAT |
| | GTGTTGAAGGTTGGCGTGTACCTGGAGAAT |
| | GGTTGGGAAAGGGGGGAAATTGCCAACGCC |
| | ATACGCCGGGTAATGGTGGACGAGGAAGGT |
| | GAGTACATACGTCAGAACGCTCGGGTTTTA |
| | AAACAAAAAGCGGACGTCAGCCTTATGAAG |
| | GGAGGTAGCTCCTATGAATCCCTAGAATCC |
| | TTGGTAAGCTATATATCTTCGTTATAA |
| | (SEQ ID NO: 17) |
| UGT76G1 | ATGAACTGGCAAATCCTGAAAGAAATCCTG |
| CP1 DNA | GGTAAAATGATCAAACAAACCAAAGCGTCG |
| | TCGGGCGTTATCTGGAACTCCTTCAAAGAA |
| | CTGGAAGAATCAGAACTGGAAACCGTTATT |
| | CGCGAAATCCCGGCTCCGTCGTTCCTGATT |
| | CCGCTGCCGAAACATCTGACCGCGAGCAGC |
| | AGCAGCCTGCTGGATCACGACCGTACGGTC |
| | TTTCAGTGGCTGGATCAGCAACCGCCGTCA |
| | TCGGTGCTGTATGTTTCATTCGGTAGCACC |
| | TCTGAAGTCGATGAAAAAGACTTTCTGGAA |
| | ATCGCTCGCGGCCTGGTGGATAGTAAACAG |
| | TCCTTCCTGTGGGTGGTTCGTCCGGGTTTT |
| | GTGAAAGGCAGCACGTGGGTTGAACCGCTG |
| | CCGGATGGCTTCCTGGGTGAACGCGGCCGT |
| | ATTGTCAAATGGGTGCCGCAGCAAGAAGTG |
| | CTGGCACATGGTGCTATCGGCGCGTTTTGG |
| | ACCCACTCTGGTTGGAACAGTACGCTGGAA |
| | TCCGTTTGCGAAGGTGTCCCGATGATTTTC |
| | AGCGATTTTGGCCTGGACCAGCCGCTGAAT |
| | GCCCGCTATATGTCTGATGTTCTGAAAGTC |
| | GGTGTGTACCTGGAAAACGGTTGGGAACGT |
| | GGCGAAATTGCGAATGCCATCCGTCGCGTT |
| | ATGGTCGATGAAGAAGGCGAATACATTCGC |
| | CAGAACGCTCGTGTCCTGAAACAAAAAGCG |
| | GACGTGAGCCTGATGAAAGGCGGTAGCTCT |
| | TATGAATCACTGGAATCGCTGGTTAGCTAC |
| | ATCAGTTCCCTGGAAAATAAAACCGAAACC |
| | ACGGTGCGTCGCCGTCGCCGTATTATCCTG |
| | TTCCCGGTTCCGTTTCAGGGTCATATTAAC |
| | CCGATCCTGCAACTGGCGAATGTTCTGTAT |
| | TCAAAAGGCTTTTCGATCACCATCTTCCAT |
| | ACGAACTTCAACAAACCGAAAACCAGTAAC |
| | TACCCGCACTTTACGTTCCGCTTTATTCTG |
| | GATAACGACCCGCAGGATGAACGTATCTCC |
| | AATCTGCCGACCCACGGCCCGCTGGCCGGT |
| | ATGCGCATTCCGATTATCAATGAACACGGT |
| | GCAGATGAACTGCGCCGTGAACTGGAACTG |
| | CTGATGCTGGCCAGTGAAGAAGATGAAGAA |
| | GTGTCCTGTCTGATCACCGACGCACTGTGG |
| | TATTTCGCCCAGAGCGTTGCAGATTCTCTG |
| | AACCTGCGCCGTCTGGTCCTGATGACGTCA |
| | TCGCTGTTCAATTTTCATGCGCACGTTTCT |
| | CTGCCGCAATTTGATGAACTGGGCTACCTG |
| | GACCCGGATGACAAAACCCGTCTGGAAGAA |
| | CAAGCCAGTGGTTTTCCGATGCTGAAAGTC |
| | AAAGACATTAAATCCGCCTATTCGTAA |
| | (SEQ ID NO: 18) |
| UGT76G1 | ATGAACTGGCAAATCCTGAAAGAAATCCTG |
| CP2 DNA | GGTAAAATGATCAAACAAACCAAAGCGTCG |
| | TCGGGCGTTATCTGGAACTCCTTCAAAGAA |
| | CTGGAAGAATCAGAACTGGAAACCGTTATT |
| | CGCGAAATCCCGGCTCCGTCGTTCCTGATT |
| | CCGCTGCCGAAACATCTGACCGCGAGCAGC |
| | AGCAGCCTGCTGGATCACGACCGTACGGTC |
| | TTTCAGTGGCTGGATCAGCAACCGCCGTCA |
| | TCGGTGCTGTATGTTTCATTCGGTAGCACC |
| | TCTGAAGTCGATGAAAAAGACTTTCTGGAA |
| | ATCGCTCGCGGCCTGGTGGATAGTAAACAG |
| | TCCTTCCTGTGGGTGGTTCGTCCGGGTTTT |
| | GTGAAAGGCAGCACGTGGGTTGAACCGCTG |
| | CCGGATGGCTTCCTGGGTGAACGCGGCCGT |
| | ATTGTCAAATGGGTGCCGCAGCAAGAAGTG |
| | CTGGCACATGGTGCTATCGGCGCGTTTTGG |
| | ACCCACTCTGGTTGGAACAGTACGCTGGAA |
| | TCCGTTTGCGAAGGTGTCCCGATGATTTTC |
| | AGCGATTTTGGCCTGGACCAGCCGCTGAAT |
| | GCCCGCTATATGTCTGATGTTCTGAAAGTC |
| | GGTGTGTACCTGGAAAACGGTTGGGAACGT |
| | GGCGAAATTGCGAATGCCATCCGTCGCGTT |
| | ATGGTCGATGAAGAAGGCGAATACATTCGC |
| | CAGAACGCTCGTGTCCTGAAACAAAAAGCG |
| | GACGTGAGCCTGATGAAAGGCGGTAGCTCT |
| | TATGAATCACTGGAATCGCTGGTTAGCTAC |
| | ATCAGTTCCCTGTACAAAGATGACAGCGGT |
| | TATAGCAGCAGCTATGCGGCGGCGGCGGGT |
| | ATGGAAAATAAAACCGAAACCACGGTGCGT |
| | CGCCGTCGCCGTATTATCCTGTTCCCGGTT |
| | CCGTTTCAGGGTCATATTAACCCGATCCTG |
| | CAACTGGCGAATGTTCTGTATTCAAAAGGC |
| | TTTTCGATCACCATCTTCCATACGAACTTC |
| | AACAAACCGAAAACCAGTAACTACCCGCAC |
| | TTTACGTTCCGCTTTATTCTGGATAACGAC |
| | CCGCAGGATGAACGTATCTCCAATCTGCCG |
| | ACCCACGGCCCGCTGGCCGGTATGCGCATT |
| | CCGATTATCAATGAACACGGTGCAGATGAA |
| | CTGCGCCGTGAACTGGAACTGCTGATGCTG |
| | GCCAGTGAAGAAGATGAAGAAGTGTCCTGT |
| | CTGATCACCGACGCACTGTGGTATTTCGCC |
| | CAGAGCGTTGCAGATTCTCTGAACCTGCGC |
| | CGTCTGGTCCTGATGACGTCATCGCTGTTC |
| | AATTTTCATGCGCACGTTTCTCTGCCGCAA |
| | TTTGATGAACTGGGCTACCTGGACCCGGAT |
| | GACAAAACCCGTCTGGAAGAACAAGCCAGT |
| | GGTTTTCCGATGCTGAAAGTCAAAGACATT |
| | AAATCCGCCTATTCGTAA |
| | (SEQ ID NO: 19) |
| EUGT11 | ATGGATTCGGGTTACTCTTCCTCCTATGCG |
| WT | GCGGCTGCGGGTATGCACGTTGTTATCTGT |
| DNA | CCGTGGCTGGCTTTTGGTCACCTGCTGCCG |
| | TGCCTGGATCTGGCACAGCGTCTGGCTTCA |
| | CGCGGCCATCGTGTCAGCTTCGTGTCTACC |
| | CCGCGCAATATTTCGCGTCTGCCGCCGGTT |
| | CGTCCGGCACTGGCTCCGCTGGTTGCATTT |
| | GTCGCTCTGCCGCTGCCGCGCGTGGAAGGT |
| | CTGCCGGATGGTGCGGAAAGTACCAACGAC |
| | GTGCCGCATGATCGCCCGGACATGGTTGAA |
| | CTGCACCGTCGTGCATTCGATGGTCTGGCA |
| | GCACCGTTTTCCGAATTTCTGGGTACGGCG |
| | TGCGCCGATTGGGTGATCGTTGACGTCTTT |
| | CATCACTGGGCGGCGGCGGCGGCGCTGGAA |
| | CATAAAGTTCCGTGTGCAATGATGCTGCTG |
| | GGCTCAGCTCACATGATTGCGTCGATCGCA |
| | GACCGTCGCCTGGAACGTGCAGAAACCGAA |
| | AGTCCGGCTGCGGCCGGCCAGGGTCGCCCG |
| | GCAGCTGCGCCGACCTTCGAAGTGGCCCGC |
| | ATGAAACTGATTCGTACGAAAGGCAGCTCT |
| | GGTATGAGCCTGGCAGAACGCTTTAGTCTG |
| | ACCCTGTCCCGTAGTTCCCTGGTGGTTGGT |
| | CGCAGTTGCGTTGAATTTGAACCGGAAACC |
| | GTCCCGCTGCTGTCCACGCTGCGTGGTAAA |
| | CCGATCACCTTTCTGGGTCTGATGCCGCCG |
| | CTGCATGAAGGCCGTCGCGAAGATGGTGAA |
| | GACGCAACGGTGCGTTGGCTGGATGCACAG |
| | CCGGCTAAAAGCGTCGTGTATGTCGCCCTG |
| | GGCTCTGAAGTGCCGCTGGGTGTGGAAAAA |
| | GTTCACGAACTGGCACTGGGCCTGGAACTG |
| | GCTGGCACCCGCTTCCTGTGGGCACTGCGT |
| | AAACCGACGGGTGTGAGCGATGCGGACCTG |
| | CTGCCGGCCGGTTTTGAAGAACGTACCCGC |
| | GGCCGTGGTGTTGTCGCAACGCGTTGGGTC |
| | CCGCAAATGAGCATTCTGGCGCATGCCGCA |
| | GTGGGCGCCTTTCTGACCCACTGTGGTTGG |
| | AACAGCACGATCGAAGGCCTGATGTTTGGT |
| | CACCCGCTGATTATGCTGCCGATCTTCGGC |
| | GATCAGGGTCCGAACGCACGTCTGATTGAA |
| | GCGAAAAATGCCGGCCTGCAAGTTGCGCGC |
| | AACGATGGCGACGGTTCTTTCGACCGTGAG |
| | GGTGTGGCTGCGGCCATTCGCGCAGTGGCT |
| | GTTGAAGAAGAATCATCGAAAGTTTTTCAG |
| | GCGAAAGCCAAAAAACTGCAAGAAATCGTC |
| | GCGGATATGGCCTGCCACGAACGCTACATT |
| | GATGGTTTCATTCAGCAACTGCGCTCCTAC |
| | AAAGACTAA |
| | (SEQ ID NO: 20) |
| EUGT11 | ATGGGTAGCTCGGGCATGTCCCTGGCGGAA |
| CP1 | CGCTTTTCGCTGACGCTGAGTCGCTCATCC |
| DNA | CTGGTTGTTGGTCGCAGTTGTGTTGAATTT |
| | GAACCGGAAACCGTTCCGCTGCTGTCTACG |
| | CTGCGCGGCAAACCGATTACCTTCCTGGGT |
| | CTGATGCCGCCGCTGCATGAAGGCCGTCGC |
| | GAAGATGGTGAAGACGCCACGGTGCGTTGG |
| | CTGGATGCTCAGCCGGCGAAATCGGTGGTT |
| | TATGTCGCACTGGGCAGCGAAGTGCCGCTG |
| | GGTGTCGAAAAAGTGCACGAACTGGCCCTG |
| | GGCCTGGAACTGGCAGGCACCCGCTTTCTG |
| | TGGGCACTGCGTAAACCGACGGGCGTTAGC |
| | GATGCTGACCTGCTGCCGGCGGGTTTCGAA |
| | GAACGCACCCGCGGCCGTGGTGTCGTGGCC |
| | ACCCGTTGGGTGCCGCAAATGTCCATTCTG |
| | GCTCATGCGGCCGTTGGCGCATTTCTGACC |
| | CACTGCGGTTGGAACAGCACGATCGAAGGC |
| | CTGATGTTTGGTCATCCGCTGATTATGCTG |
| | CCGATCTTCGGCGATCAGGGTCCGAACGCA |
| | CGCCTGATCGAAGCCAAAAATGCAGGCCTG |
| | CAAGTTGCGCGTAACGATGGCGACGGTAGC |
| | TTTGACCGCGAAGGTGTCGCAGCTGCGATT |
| | CGTGCTGTGGCGGTTGAAGAAGAAAGCAGC |
| | AAAGTCTTCCAGGCCAAAGCGAAAAAACTG |
| | CAAGAAATCGTGGCTGATATGGCGTGTCAT |
| | GAACGCTATATTGACGGCTTTATCCAGCAA |
| | CTGCGTTCTTACAAAGATGACAGTGGCTAT |
| | AGTTCCTCATACGCCGCAGCTGCGGGTATG |
| | CATGTTGTCATTTGCCCGTGGCTGGCGTTT |
| | GGTCACCTGCTGCCGTGTCTGGATCTGGCA |
| | CAGCGCCTGGCATCTCGCGGTCACCGTGTT |
| | TCGTTCGTCAGCACCCCGCGCAATATCAGT |
| | CGTCTGCCGCCGGTTCGTCCGGCGCTGGCG |
| | CCGCTGGTTGCGTTCGTTGCACTGCCGCTG |
| | CCGCGTGTGGAAGGTCTGCCGGATGGTGCC |
| | GAATCGACCAACGACGTTCCGCATGATCGT |
| | CCGGACATGGTCGAACTGCATCGTCGCGCC |
| | TTTGATGGCCTGGCCGCACCGTTTAGCGAA |
| | TTTCTGGGTACGGCCTGCGCAGATTGGGTC |
| | ATTGTGGACGTTTTTCACCACTGGGCGGCG |
| | GCGGCGGCGCTGGAACATAAAGTGCCGTGT |
| | GCGATGATGCTGCTGGGTTCCGCCCACATG |
| | ATTGCTTCAATCGCGGATCGTCGCCTGGAA |
| | CGTGCCGAAACCGAAAGTCCGGCGGCGGCA |
| | GGCCAGGGTCGTCCGGCGGCGGCACCGACC |
| | TTTGAAGTGGCACGTATGAAACTGATTCGC |
| | ACGAAATAA |
| | (SEQ ID NO: 21) |
| HV1 | ATGGATGGTAACTCCTCCTCCTCGCCGCTG |
| glycosyl | CATGTGGTCATTTGTCCGTGGCTGGCTCTG |
| transferase | GGTCACCTGCTGCCGTGTCTGGATATTGCT |
| DNA | GAACGTCTGGCGTCACGCGGCCATCGTGTC |
| | AGTTTTGTGTCCACCCCGCGCAACATTGCC |
| | CGTCTGCCGCCGCTGCGTCCGGCTGTTGCA |
| | CCGCTGGTTGATTTCGTCGCACTGCCGCTG |
| | CCGCATGTTGACGGTCTGCCGGAGGGTGCG |
| | GAATCGACCAATGATGTGCCGTATGACAAA |
| | TTTGAACTGCACCGTAAGGCGTTCGATGGT |
| | CTGGCGGCCCCGTTTAGCGAATTTCTGCGT |
| | GCAGCTTGCGCAGAAGGTGCAGGTTCTCGC |
| | CCGGATTGGCTGATTGTGGACACCTTTCAT |
| | CACTGGGCGGCGGCGGCGGCGGTGGAAAAC |
| | AAAGTGCCGTGTGTTATGCTGCTGCTGGGT |
| | GCAGCAACGGTGATCGCTGGTTTCGCGCGT |
| | GGTGTTAGCGAACATGCGGCGGCGGCGGTG |
| | GGTAAAGAACGTCCGGCTGCGGAAGCCCCG |
| | AGTTTTGAAACCGAACGTCGCAAGCTGATG |
| | ACCACGCAGAATGCCTCCGGCATGACCGTG |
| | GCAGAACGCTATTTCCTGACGCTGATGCGT |
| | AGCGATCTGGTTGCCATCCGCTCTTGCGCA |
| | GAATGGGAACCGGAAAGCGTGGCAGCACTG |
| | ACCACGCTGGCAGGTAAACCGGTGGTTCCG |
| | CTGGGTCTGCTGCCGCCGAGTCCGGAAGGC |
| | GGTCGTGGCGTTTCCAAAGAAGATGCTGCG |
| | GTCCGTTGGCTGGACGCACAGCCGGCAAAG |
| | TCAGTCGTGTACGTCGCACTGGGTTCGGAA |
| | GTGCCGCTGCGTGCGGAACAAGTTCACGAA |
| | CTGGCACTGGGCCTGGAACTGAGCGGTGCT |
| | CGCTTTCTGTGGGCGCTGCGTAAACCGACC |
| | GATGCACCGGACGCCGCAGTGCTGCCGCCG |
| | GGTTTCGAAGAACGTACCCGCGGCCGTGGT |
| | CTGGTTGTCACGGGTTGGGTGCCGCAGATT |
| | GGCGTTCTGGCTCATGGTGCGGTGGCTGCG |
| | TTTCTGACCCACTGTGGCTGGAACTCTACG |
| | ATCGAAGGCCTGCTGTTCGGTCATCCGCTG |
| | ATTATGCTGCCGATCAGCTCTGATCAGGGT |
| | CCGAATGCGCGCCTGATGGAAGGCCGTAAA |
| | GTCGGTATGCAAGTGCCGCGTGATGAATCA |
| | GACGGCTCGTTTCGTCGCGAAGATGTTGCC |
| | GCAACCGTCCGCGCCGTGGCAGTTGAAGAA |
| | GACGGTCGTCGCGTCTTCACGGCTAACGCG |
| | AAAAAGATGCAAGAAATTGTGGCCGATGGC |
| | GCATGCCACGAACGTTGTATTGACGGTTTT |
| | ATCCAGCAACTGCGCAGTTACAAGGCGTAA |
| | (SEQ ID NO: 22) |
| SUS1 WT | ATGGCAAACGCTGAACGTATGATTACCCGT |
| from | GTCCACTCCCAACGCGAACGCCTGAACGAA |
| Arabidopsis | ACCCTGGTGTCGGAACGCAACGAAGTTCTG |
| thaliana, | GCACTGCTGAGCCGTGTGGAAGCTAAGGGC |
| DNA | AAAGGTATTCTGCAGCAAAACCAGATTATC |
| | GCGGAATTTGAAGCCCTGCCGGAACAAACC |
| | CGCAAAAAGCTGGAAGGCGGTCCGTTTTTC |
| | GATCTGCTGAAATCTACGCAGGAAGCGATC |
| | GTTCTGCCGCCGTGGGTCGCACTGGCAGTG |
| | CGTCCGCGTCCGGGCGTTTGGGAATATCTG |
| | CGTGTCAACCTGCATGCACTGGTGGTTGAA |
| | GAACTGCAGCCGGCTGAATTTCTGCACTTC |
| | AAGGAAGAACTGGTTGACGGCGTCAAAAAC |
| | GGTAATTTTACCCTGGAACTGGATTTTGAA |
| | CCGTTCAATGCCAGTATCCCGCGTCCGACG |
| | CTGCATAAATATATTGGCAACGGTGTGGAC |
| | TTTCTGAATCGCCATCTGAGCGCAAAGCTG |
| | TTCCACGATAAAGAATCTCTGCTGCCGCTG |
| | CTGAAATTCCTGCGTCTGCATAGTCACCAG |
| | GGCAAGAACCTGATGCTGTCCGAAAAAATT |
| | CAGAACCTGAATACCCTGCAACACACGCTG |
| | CGCAAGGCGGAAGAATACCTGGCCGAACTG |
| | AAAAGTGAAACCCTGTACGAAGAATTCGAA |
| | GCAAAGTTCGAAGAAATTGGCCTGGAACGT |
| | GGCTGGGGTGACAATGCTGAACGTGTTCTG |
| | GATATGATCCGTCTGCTGCTGGACCTGCTG |
| | GAAGCACCGGACCCGTGCACCCTGGAAACG |
| | TTTCTGGGTCGCGTGCCGATGGTTTTCAAC |
| | GTCGTGATTCTGTCCCCGCATGGCTATTTT |
| | GCACAGGACAATGTGCTGGGTTACCCGGAT |
| | ACCGGCGGTCAGGTTGTCTATATTCTGGAT |
| | CAAGTTCGTGCGCTGGAAATTGAAATGCTG |
| | CAGCGCATCAAGCAGCAAGGCCTGAACATC |
| | AAACCGCGTATTCTGATCCTGACCCGTCTG |
| | CTGCCGGATGCAGTTGGTACCACGTGCGGT |
| | GAACGTCTGGAACGCGTCTATGACAGCGAA |
| | TACTGTGATATTCTGCGTGTCCCGTTTCGC |
| | ACCGAAAAGGGTATTGTGCGTAAATGGATC |
| | AGTCGCTTCGAAGTTTGGCCGTATCTGGAA |
| | ACCTACACGGAAGATGCGGCCGTGGAACTG |
| | TCCAAGGAACTGAATGGCAAACCGGACCTG |
| | ATTATCGGCAACTATAGCGATGGTAATCTG |
| | GTCGCATCTCTGCTGGCTCATAAACTGGGT |
| | GTGACCCAGTGCACGATTGCACACGCTCTG |
| | GAAAAGACCAAATATCCGGATTCAGACATC |
| | TACTGGAAAAAGCTGGATGACAAATATCAT |
| | TTTTCGTGTCAGTTCACCGCGGACATTTTT |
| | GCCATGAACCACACGGATTTTATTATCACC |
| | AGTACGTTCCAGGAAATCGCGGGCTCCAAA |
| | GAAACCGTGGGTCAATACGAATCACATACC |
| | GCCTTCACGCTGCCGGGCCTGTATCGTGTG |
| | GTTCACGGTATCGATGTTTTTGACCCGAAA |
| | TTCAATATTGTCAGTCCGGGCGCGGATATG |
| | TCCATCTATTTTCCGTACACCGAAGAAAAG |
| | CGTCGCCTGACGAAATTCCATTCAGAAATT |
| | GAAGAACTGCTGTACTCGGACGTGGAAAAC |
| | AAGGAACACCTGTGTGTTCTGAAAGATAAA |
| | AAGAAACCGATCCTGTTTACCATGGCCCGT |
| | CTGGATCGCGTGAAGAATCTGTCAGGCCTG |
| | GTTGAATGGTATGGTAAAAACACGCGTCTG |
| | CGCGAACTGGCAAATCTGGTCGTGGTTGGC |
| | GGTGACCGTCGCAAGGAATCGAAAGATAAC |
| | GAAGAAAAGGCTGAAATGAAGAAAATGTAC |
| | GATCTGATCGAAGAATACAAGCTGAACGGC |
| | CAGTTTCGTTGGATCAGCTCTCAAATGGAC |
| | CGTGTGCGCAATGGCGAACTGTATCGCTAC |
| | ATTTGCGATACCAAGGGTGCGTTTGTTCAG |
| | CCGGCACTGTACGAAGCTTTCGGCCTGACC |
| | GTCGTGGAAGCCATGACGTGCGGTCTGCCG |
| | ACCTTTGCGACGTGTAAAGGCGGTCCGGCC |
| | GAAATTATCGTGCATGGCAAATCTGGTTTC |
| | CATATCGATCCGTATCACGGTGATCAGGCA |
| | GCTGACACCCTGGCGGATTTCTTTACGAAG |
| | TGTAAAGAAGACCCGTCACACTGGGATGAA |
| | ATTTCGAAGGGCGGTCTGCAACGTATCGAA |
| | GAAAAATATACCTGGCAGATTTACAGCCAA |
| | CGCCTGCTGACCCTGACGGGCGTCTACGGT |
| | TTTTGGAAACATGTGTCTAATCTGGATCGC |
| | CTGGAAGCCCGTCGCTATCTGGAAATGTTT |
| | TACGCACTGAAGTATCGCCCGCTGGCACAA |
| | GCCGTTCCGCTGGCACAGGACGACTAA |
| | (SEQ ID NO: 23) |
| UGT76G1- | ATGGAGAATAAGACAGAAACAACCGTAAGA |
| AtSUS1 | CGGAGGCGGAGGATTATCTTGTTCCCTGTA |
| fusion | CCATTTCAGGGCCATATTAATCCGATCCTC |
| enzyme, | CAATTAGCAAACGTCCTCTACTCCAAGGGA |
| DNA | TTTTCAATAACAATCTTCCATACTAACTTT |
| | AACAAGCCTAAAACGAGTAATTATCCTCAC |
| | TTTACATTCAGGTTCATTCTAGACAACGAC |
| | CCTCAGGATGAGCGTATCTCAAATTTACCT |
| | ACGCATGGCCCCTTGGCAGGTATGCGAATA |
| | CCAATAATCAATGAGCATGGAGCCGATGAA |
| | CTCCGTCGCGAGTTAGAGCTTCTCATGCTC |
| | GCAAGTGAGGAAGACGAGGAAGTTTCGTGC |
| | CTAATAACTGATGCGCTTTGGTACTTCGCC |
| | CAATCAGTCGCAGACTCACTGAATCTACGC |
| | CGTTTGGTCCTTATGACAAGTTCATTATTC |
| | AACTTTCACGCACATGTATCACTGCCGCAA |
| | TTTGACGAGTTGGGTTACCTGGACCCGGAT |
| | GACAAAACGCGATTGGAGGAACAAGCGTCG |
| | GGCTTCCCCATGCTGAAAGTCAAAGATATT |
| | AAGAGCGCTTATAGTAATTGGCAAATTCTG |
| | AAAGAAATTCTCGGAAAAATGATAAAGCAA |
| | ACCAAAGCGTCCTCTGGAGTAATCTGGAAC |
| | TCCTTCAAGGAGTTAGAGGAATCTGAACTT |
| | GAAACGGTCATCAGAGAAATCCCCGCTCCC |
| | TCGTTCTTAATTCCACTACCCAAGCACCTT |
| | ACTGCAAGTAGCAGTTCCCTCCTAGATCAT |
| | GACCGAACCGTGTTTCAGTGGCTGGATCAG |
| | CAACCCCCGTCGTCAGTTCTATATGTAAGC |
| | TTTGGGAGTACTTCGGAAGTGGATGAAAAG |
| | GACTTCTTAGAGATTGCGCGAGGGCTCGTG |
| | GATAGCAAACAGAGCTTCCTGTGGGTAGTG |
| | AGACCGGGATTCGTTAAGGGCTCGACGTGG |
| | GTCGAGCCGTTGCCAGATGGTTTTCTAGGG |
| | GAGAGAGGGAGAATCGTGAAATGGGTTCCA |
| | CAGCAAGAGGTTTTGGCTCACGGAGCTATA |
| | GGGGCCTTTTGGACCCACTCTGGTTGGAAT |
| | TCTACTCTTGAAAGTGTCTGTGAAGGCGTT |
| | CCAATGATATTTTCTGATTTTGGGCTTGAC |
| | CAGCCTCTAAACGCTCGCTATATGTCTGAT |
| | GTGTTGAAGGTTGGCGTGTACCTGGAGAAT |
| | GGTTGGGAAAGGGGGGAAATTGCCAACGCC |
| | ATACGCCGGGTAATGGTGGACGAGGAAGGT |
| | GAGTACATACGTCAGAACGCTCGGGTTTTA |
| | AAACAAAAAGCGGACGTCAGCCTTATGAAG |
| | GGAGGTAGCTCCTATGAATCCCTAGAATCC |
| | TTGGTAAGCTATATATCTTCGTTAGGTTCT |
| | GGTGCAAACGCTGAACGTATGATAACGCGC |
| | GTCCACAGCCAACGTGAGCGTTTGAACGAA |
| | ACGCTTGTTTCTGAGAGAAACGAAGTCCTT |
| | GCCTTGCTTTCCAGGGTTGAAGCCAAAGGT |
| | AAAGGTATTTTACAACAAAACCAGATCATT |
| | GCTGAATTCGAAGCTTTGCCTGAACAAACC |
| | CGGAAGAAACTTGAAGGTGGTCCTTTCTTT |
| | GACCTTCTCAAATCCACTCAGGAAGCAATT |
| | GTGTTGCCACCATGGGTTGCTCTAGCTGTG |
| | AGGCCAAGGCCTGGTGTTTGGGAATACTTA |
| | CGAGTCAATCTCCATGCTCTTGTCGTTGAA |
| | GAACTCCAACCTGCTGAGTTTCTTCATTTC |
| | AAGGAAGAACTCGTTGATGGAGTTAAGAAT |
| | GGTAATTTCACTCTTGAGCTTGATTTCGAG |
| | CCATTCAATGCGTCTATCCCTCGTCCAACA |
| | CTCCACAAATACATTGGAAATGGTGTTGAC |
| | TTCCTTAACCGTCATTTATCGGCTAAGCTC |
| | TTCCATGACAAGGAGAGTTTGCTTCCATTG |
| | CTTAAGTTCCTTCGTCTTCACAGCCACCAG |
| | GGCAAGAACCTGATGTTGAGCGAGAAGATT |
| | CAGAACCTCAACACTCTGCAACACACCTTG |
| | AGGAAAGCAGAAGAGTATCTAGCAGAGCTT |
| | AAGTCCGAAACACTGTATGAAGAGTTTGAG |
| | GCCAAGTTTGAGGAGATTGGTCTTGAGAGG |
| | GGATGGGGAGACAATGCAGAGCGTGTCCTT |
| | GACATGATACGTCTTCTTTTGGACCTTCTT |
| | GAGGCGCCTGATCCTTGCACTCTTGAGACT |
| | TTTCTTGGAAGAGTACCAATGGTGTTCAAC |
| | GTTGTGATCCTCTCTCCACATGGTTACTTT |
| | GCTCAGGACAATGTTCTTGGTTACCCTGAC |
| | ACTGGTGGACAGGTTGTTTACATTCTTGAT |
| | CAAGTTCGTGCTCTGGAGATAGAGATGCTT |
| | CAACGTATTAAGCAACAAGGACTCAACATT |
| | AAACCAAGGATTCTCATTCTAACTCGACTT |
| | CTACCTGATGCGGTAGGAACTACATGCGGT |
| | GAACGTCTCGAGAGAGTTTATGATTCTGAG |
| | TACTGTGATATTCTTCGTGTGCCCTTCAGA |
| | ACAGAGAAGGGTATTGTTCGCAAATGGATC |
| | TCAAGGTTCGAAGTCTGGCCATATCTAGAG |
| | ACTTACACCGAGGATGCTGCGGTTGAGCTA |
| | TCGAAAGAATTGAATGGCAAGCCTGACCTT |
| | ATCATTGGTAACTACAGTGATGGAAATCTT |
| | GTTGCTTCTTTATTGGCTCACAAACTTGGT |
| | GTCACTCAGTGTACCATTGCTCATGCTCTT |
| | GAGAAAACAAAGTACCCGGATTCTGATATC |
| | TACTGGAAGAAGCTTGACGACAAGTACCAT |
| | TTCTCATGCCAGTTCACTGCGGATATTTTC |
| | GCAATGAACCACACTGATTTCATCATCACT |
| | AGTACTTTCCAAGAAATTGCTGGAAGCAAA |
| | GAAACTGTTGGGCAGTATGAAAGCCACACA |
| | GCCTTTACTCTTCCCGGATTGTATCGAGTT |
| | GTTCACGGGATTGATGTGTTTGATCCCAAG |
| | TTCAACATTGTCTCTCCTGGTGCTGATATG |
| | AGCATCTACTTCCCTTACACAGAGGAGAAG |
| | CGTAGATTGACTAAGTTCCACTCTGAGATC |
| | GAGGAGCTCCTCTACAGCGATGTTGAGAAC |
| | AAAGAGCACTTATGTGTGCTCAAGGACAAG |
| | AAGAAGCCGATTCTCTTCACAATGGCTAGG |
| | CTTGATCGTGTCAAGAACTTGTCAGGTCTT |
| | GTTGAGTGGTACGGGAAGAACACCCGCTTG |
| | CGTGAGCTAGCTAACTTGGTTGTTGTTGGA |
| | GGAGACAGGAGGAAAGAGTCAAAGGACAAT |
| | GAAGAGAAAGCAGAGATGAAGAAAATGTAT |
| | GATCTCATTGAGGAATACAAGCTAAACGGT |
| | CAGTTCAGGTGGATCTCCTCTCAGATGGAC |
| | CGGGTAAGGAACGGTGAGCTGTACCGGTAC |
| | ATCTGTGACACCAAGGGTGCTTTTGTCCAA |
| | CCTGCATTATATGAAGCCTTTGGGTTAACT |
| | GTTGTGGAGGCTATGACTTGTGGTTTACCG |
| | ACTTTCGCCACTTGCAAAGGTGGTCCAGCT |
| | GAGATCATTGTGCACGGTAAATCGGGTTTC |
| | CACATTGACCCTTACCATGGTGATCAGGCT |
| | GCTGATACTCTTGCTGATTTCTTCACCAAG |
| | TGTAAGGAGGATCCATCTCACTGGGATGAG |
| | ATCTCAAAAGGAGGGCTTCAGAGGATTGAG |
| | GAGAAATACACTTGGCAAATCTATTCACAG |
| | AGGCTCTTGACATTGACTGGTGTGTATGGA |
| | TTCTGGAAGCATGTCTCGAACCTTGACCGT |
| | CTTGAGGCTCGCCGTTACCTTGAAATGTTC |
| | TATGCATTGAAGTATCGCCCATTGGCTCAG |
| | GCTGTTCCTCTTGCACAAGATGATTGA |
| | (SEQ ID NO: 24) |
| EUGT11- | ATGGATTCGGGTTACTCTTCCTCCTATGCG |
| AtSUS1 | GCGGCTGCGGGTATGCACGTTGTTATCTGT |
| fusion | CCGTGGCTGGCTTTTGGTCACCTGCTGCCG |
| enzyme, | TGCCTGGATCTGGCACAGCGTCTGGCTTCA |
| DNA | CGCGGCCATCGTGTCAGCTTCGTGTCTACC |
| | CCGCGCAATATTTCGCGTCTGCCGCCGGTT |
| | CGTCCGGCACTGGCTCCGCTGGTTGCATTT |
| | GTCGCTCTGCCGCTGCCGCGCGTGGAAGGT |
| | CTGCCGGATGGTGCGGAAAGTACCAACGAC |
| | GTGCCGCATGATCGCCCGGACATGGTTGAA |
| | CTGCACCGTCGTGCATTCGATGGTCTGGCA |
| | GCACCGTTTTCCGAATTTCTGGGTACGGCG |
| | TGCGCCGATTGGGTGATCGTTGACGTCTTT |
| | CATCACTGGGCGGCGGCGGCGGCGCTGGAA |
| | CATAAAGTTCCGTGTGCAATGATGCTGCTG |
| | GGCTCAGCTCACATGATTGCGTCGATCGCA |
| | GACCGTCGCCTGGAACGTGCAGAAACCGAA |
| | AGTCCGGCTGCGGCCGGCCAGGGTCGCCCG |
| | GCAGCTGCGCCGACCTTCGAAGTGGCCCGC |
| | ATGAAACTGATTCGTACGAAAGGCAGCTCT |
| | GGTATGAGCCTGGCAGAACGCTTTAGTCTG |
| | ACCCTGTCCCGTAGTTCCCTGGTGGTTGGT |
| | CGCAGTTGCGTTGAATTTGAACCGGAAACC |
| | GTCCCGCTGCTGTCCACGCTGCGTGGTAAA |
| | CCGATCACCTTTCTGGGTCTGATGCCGCCG |
| | CTGCATGAAGGCCGTCGCGAAGATGGTGAA |
| | GACGCAACGGTGCGTTGGCTGGATGCACAG |
| | CCGGCTAAAAGCGTCGTGTATGTCGCCCTG |
| | GGCTCTGAAGTGCCGCTGGGTGTGGAAAAA |
| | GTTCACGAACTGGCACTGGGCCTGGAACTG |
| | GCTGGCACCCGCTTCCTGTGGGCACTGCGT |
| | AAACCGACGGGTGTGAGCGATGCGGACCTG |
| | CTGCCGGCCGGTTTTGAAGAACGTACCCGC |
| | GGCCGTGGTGTTGTCGCAACGCGTTGGGTC |
| | CCGCAAATGAGCATTCTGGCGCATGCCGCA |
| | GTGGGCGCCTTTCTGACCCACTGTGGTTGG |
| | AACAGCACGATCGAAGGCCTGATGTTTGGT |
| | CACCCGCTGATTATGCTGCCGATCTTCGGC |
| | GATCAGGGTCCGAACGCACGTCTGATTGAA |
| | GCGAAAAATGCCGGCCTGCAAGTTGCGCGC |
| | AACGATGGCGACGGTTCTTTCGACCGTGAG |
| | GGTGTGGCTGCGGCCATTCGCGCAGTGGCT |
| | GTTGAAGAAGAATCATCGAAAGTTTTTCAG |
| | GCGAAAGCCAAAAAACTGCAAGAAATCGTC |
| | GCGGATATGGCCTGCCACGAACGCTACATT |
| | GATGGTTTCATTCAGCAACTGCGCTCCTAC |
| | AAAGACGGTTCTGGTGCAAACGCTGAACGT |
| | ATGATAACGCGCGTCCACAGCCAACGTGAG |
| | CGTTTGAACGAAACGCTTGTTTCTGAGAGA |
| | AACGAAGTCCTTGCCTTGCTTTCCAGGGTT |
| | GAAGCCAAAGGTAAAGGTATTTTACAACAA |
| | AACCAGATCATTGCTGAATTCGAAGCTTTG |
| | CCTGAACAAACCCGGAAGAAACTTGAAGGT |
| | GGTCCTTTCTTTGACCTTCTCAAATCCACT |
| | CAGGAAGCAATTGTGTTGCCACCATGGGTT |
| | GCTCTAGCTGTGAGGCCAAGGCCTGGTGTT |
| | TGGGAATACTTACGAGTCAATCTCCATGCT |
| | CTTGTCGTTGAAGAACTCCAACCTGCTGAG |
| | TTTCTTCATTTCAAGGAAGAACTCGTTGAT |
| | GGAGTTAAGAATGGTAATTTCACTCTTGAG |
| | CTTGATTTCGAGCCATTCAATGCGTCTATC |
| | CCTCGTCCAACACTCCACAAATACATTGGA |
| | AATGGTGTTGACTTCCTTAACCGTCATTTA |
| | TCGGCTAAGCTCTTCCATGACAAGGAGAGT |
| | TTGCTTCCATTGCTTAAGTTCCTTCGTCTT |
| | CACAGCCACCAGGGCAAGAACCTGATGTTG |
| | AGCGAGAAGATTCAGAACCTCAACACTCTG |
| | CAACACACCTTGAGGAAAGCAGAAGAGTAT |
| | CTAGCAGAGCTTAAGTCCGAAACACTGTAT |
| | GAAGAGTTTGAGGCCAAGTTTGAGGAGATT |
| | GGTCTTGAGAGGGGATGGGGAGACAATGCA |
| | GAGCGTGTCCTTGACATGATACGTCTTCTT |
| | TTGGACCTTCTTGAGGCGCCTGATCCTTGC |
| | ACTCTTGAGACTTTTCTTGGAAGAGTACCA |
| | ATGGTGTTCAACGTTGTGATCCTCTCTCCA |
| | CATGGTTACTTTGCTCAGGACAATGTTCTT |
| | GGTTACCCTGACACTGGTGGACAGGTTGTT |
| | TACATTCTTGATCAAGTTCGTGCTCTGGAG |
| | ATAGAGATGCTTCAACGTATTAAGCAACAA |
| | GGACTCAACATTAAACCAAGGATTCTCATT |
| | CTAACTCGACTTCTACCTGATGCGGTAGGA |
| | ACTACATGCGGTGAACGTCTCGAGAGAGTT |
| | TATGATTCTGAGTACTGTGATATTCTTCGT |
| | GTGCCCTTCAGAACAGAGAAGGGTATTGTT |
| | CGCAAATGGATCTCAAGGTTCGAAGTCTGG |
| | CCATATCTAGAGACTTACACCGAGGATGCT |
| | GCGGTTGAGCTATCGAAAGAATTGAATGGC |
| | AAGCCTGACCTTATCATTGGTAACTACAGT |
| | GATGGAAATCTTGTTGCTTCTTTATTGGCT |
| | CACAAACTTGGTGTCACTCAGTGTACCATT |
| | GCTCATGCTCTTGAGAAAACAAAGTACCCG |
| | GATTCTGATATCTACTGGAAGAAGCTTGAC |
| | GACAAGTACCATTTCTCATGCCAGTTCACT |
| | GCGGATATTTTCGCAATGAACCACACTGAT |
| | TTCATCATCACTAGTACTTTCCAAGAAATT |
| | GCTGGAAGCAAAGAAACTGTTGGGCAGTAT |
| | GAAAGCCACACAGCCTTTACTCTTCCCGGA |
| | TTGTATCGAGTTGTTCACGGGATTGATGTG |
| | TTTGATCCCAAGTTCAACATTGTCTCTCCT |
| | GGTGCTGATATGAGCATCTACTTCCCTTAC |
| | ACAGAGGAGAAGCGTAGATTGACTAAGTTC |
| | CACTCTGAGATCGAGGAGCTCCTCTACAGC |
| | GATGTTGAGAACAAAGAGCACTTATGTGTG |
| | CTCAAGGACAAGAAGAAGCCGATTCTCTTC |
| | ACAATGGCTAGGCTTGATCGTGTCAAGAAC |
| | TTGTCAGGTCTTGTTGAGTGGTACGGGAAG |
| | AACACCCGCTTGCGTGAGCTAGCTAACTTG |
| | GTTGTTGTTGGAGGAGACAGGAGGAAAGAG |
| | TCAAAGGACAATGAAGAGAAAGCAGAGATG |
| | AAGAAAATGTATGATCTCATTGAGGAATAC |
| | AAGCTAAACGGTCAGTTCAGGTGGATCTCC |
| | TCTCAGATGGACCGGGTAAGGAACGGTGAG |
| | CTGTACCGGTACATCTGTGACACCAAGGGT |
| | GCTTTTGTCCAACCTGCATTATATGAAGCC |
| | TTTGGGTTAACTGTTGTGGAGGCTATGACT |
| | TGTGGTTTACCGACTTTCGCCACTTGCAAA |
| | GGTGGTCCAGCTGAGATCATTGTGCACGGT |
| | AAATCGGGTTTCCACATTGACCCTTACCAT |
| | GGTGATCAGGCTGCTGATACTCTTGCTGAT |
| | TTCTTCACCAAGTGTAAGGAGGATCCATCT |
| | CACTGGGATGAGATCTCAAAAGGAGGGCTT |
| | CAGAGGATTGAGGAGAAATACACTTGGCAA |
| | ATCTATTCACAGAGGCTCTTGACATTGACT |
| | GGTGTGTATGGATTCTGGAAGCATGTCTCG |
| | AACCTTGACCGTCTTGAGGCTCGCCGTTAC |
| | CTTGAAATGTTCTATGCATTGAAGTATCGC |
| | CCATTGGCTCAGGCTGTTCCTCTTGCACAA |
| | GATGATTGA |
| | (SEQ ID NO: 25) |
| HV1- | ATGGATGGTAACTCCTCCTCCTCGCCGCTG |
| AtSUS1 | CATGTGGTCATTTGTCCGTGGCTGGCTCTG |
| fusion | GGTCACCTGCTGCCGTGTCTGGATATTGCT |
| enzyme, | GAACGTCTGGCGTCACGCGGCCATCGTGTC |
| DNA | AGTTTTGTGTCCACCCCGCGCAACATTGCC |
| | CGTCTGCCGCCGCTGCGTCCGGCTGTTGCA |
| | CCGCTGGTTGATTTCGTCGCACTGCCGCTG |
| | CCGCATGTTGACGGTCTGCCGGAGGGTGCG |
| | GAATCGACCAATGATGTGCCGTATGACAAA |
| | TTTGAACTGCACCGTAAGGCGTTCGATGGT |
| | CTGGCGGCCCCGTTTAGCGAATTTCTGCGT |
| | GCAGCTTGCGCAGAAGGTGCAGGTTCTCGC |
| | CCGGATTGGCTGATTGTGGACACCTTTCAT |
| | CACTGGGCGGCGGCGGCGGCGGTGGAAAAC |
| | AAAGTGCCGTGTGTTATGCTGCTGCTGGGT |
| | GCAGCAACGGTGATCGCTGGTTTCGCGCGT |
| | GGTGTTAGCGAACATGCGGCGGCGGCGGTG |
| | GGTAAAGAACGTCCGGCTGCGGAAGCCCCG |
| | AGTTTTGAAACCGAACGTCGCAAGCTGATG |
| | ACCACGCAGAATGCCTCCGGCATGACCGTG |
| | GCAGAACGCTATTTCCTGACGCTGATGCGT |
| | AGCGATCTGGTTGCCATCCGCTCTTGCGCA |
| | GAATGGGAACCGGAAAGCGTGGCAGCACTG |
| | ACCACGCTGGCAGGTAAACCGGTGGTTCCG |
| | CTGGGTCTGCTGCCGCCGAGTCCGGAAGGC |
| | GGTCGTGGCGTTTCCAAAGAAGATGCTGCG |
| | GTCCGTTGGCTGGACGCACAGCCGGCAAAG |
| | TCAGTCGTGTACGTCGCACTGGGTTCGGAA |
| | GTGCCGCTGCGTGCGGAACAAGTTCACGAA |
| | CTGGCACTGGGCCTGGAACTGAGCGGTGCT |
| | CGCTTTCTGTGGGCGCTGCGTAAACCGACC |
| | GATGCACCGGACGCCGCAGTGCTGCCGCCG |
| | GGTTTCGAAGAACGTACCCGCGGCCGTGGT |
| | CTGGTTGTCACGGGTTGGGTGCCGCAGATT |
| | GGCGTTCTGGCTCATGGTGCGGTGGCTGCG |
| | TTTCTGACCCACTGTGGCTGGAACTCTACG |
| | ATCGAAGGCCTGCTGTTCGGTCATCCGCTG |
| | ATTATGCTGCCGATCAGCTCTGATCAGGGT |
| | CCGAATGCGCGCCTGATGGAAGGCCGTAAA |
| | GTCGGTATGCAAGTGCCGCGTGATGAATCA |
| | GACGGCTCGTTTCGTCGCGAAGATGTTGCC |
| | GCAACCGTCCGCGCCGTGGCAGTTGAAGAA |
| | GACGGTCGTCGCGTCTTCACGGCTAACGCG |
| | AAAAAGATGCAAGAAATTGTGGCCGATGGC |
| | GCATGCCACGAACGTTGTATTGACGGTTTT |
| | ATCCAGCAACTGCGCAGTTACAAGGCGGGT |
| | TCTGGTGCAAACGCTGAACGTATGATAACG |
| | CGCGTCCACAGCCAACGTGAGCGTTTGAAC |
| | GAAACGCTTGTTTCTGAGAGAAACGAAGTC |
| | CTTGCCTTGCTTTCCAGGGTTGAAGCCAAA |
| | GGTAAAGGTATTTTACAACAAAACCAGATC |
| | ATTGCTGAATTCGAAGCTTTGCCTGAACAA |
| | ACCCGGAAGAAACTTGAAGGTGGTCCTTTC |
| | TTTGACCTTCTCAAATCCACTCAGGAAGCA |
| | ATTGTGTTGCCACCATGGGTTGCTCTAGCT |
| | GTGAGGCCAAGGCCTGGTGTTTGGGAATAC |
| | TTACGAGTCAATCTCCATGCTCTTGTCGTT |
| | GAAGAACTCCAACCTGCTGAGTTTCTTCAT |
| | TTCAAGGAAGAACTCGTTGATGGAGTTAAG |
| | AATGGTAATTTCACTCTTGAGCTTGATTTC |
| | GAGCCATTCAATGCGTCTATCCCTCGTCCA |
| | ACACTCCACAAATACATTGGAAATGGTGTT |
| | GACTTCCTTAACCGTCATTTATCGGCTAAG |
| | CTCTTCCATGACAAGGAGAGTTTGCTTCCA |
| | TTGCTTAAGTTCCTTCGTCTTCACAGCCAC |
| | CAGGGCAAGAACCTGATGTTGAGCGAGAAG |
| | ATTCAGAACCTCAACACTCTGCAACACACC |
| | TTGAGGAAAGCAGAAGAGTATCTAGCAGAG |
| | CTTAAGTCCGAAACACTGTATGAAGAGTTT |
| | GAGGCCAAGTTTGAGGAGATTGGTCTTGAG |
| | AGGGGATGGGGAGACAATGCAGAGCGTGTC |
| | CTTGACATGATACGTCTTCTTTTGGACCTT |
| | CTTGAGGCGCCTGATCCTTGCACTCTTGAG |
| | ACTTTTCTTGGAAGAGTACCAATGGTGTTC |
| | AACGTTGTGATCCTCTCTCCACATGGTTAC |
| | TTTGCTCAGGACAATGTTCTTGGTTACCCT |
| | GACACTGGTGGACAGGTTGTTTACATTCTT |
| | GATCAAGTTCGTGCTCTGGAGATAGAGATG |
| | CTTCAACGTATTAAGCAACAAGGACTCAAC |
| | ATTAAACCAAGGATTCTCATTCTAACTCGA |
| | CTTCTACCTGATGCGGTAGGAACTACATGC |
| | GGTGAACGTCTCGAGAGAGTTTATGATTCT |
| | GAGTACTGTGATATTCTTCGTGTGCCCTTC |
| | AGAACAGAGAAGGGTATTGTTCGCAAATGG |
| | ATCTCAAGGTTCGAAGTCTGGCCATATCTA |
| | GAGACTTACACCGAGGATGCTGCGGTTGAG |
| | CTATCGAAAGAATTGAATGGCAAGCCTGAC |
| | CTTATCATTGGTAACTACAGTGATGGAAAT |
| | CTTGTTGCTTCTTTATTGGCTCACAAACTT |
| | GGTGTCACTCAGTGTACCATTGCTCATGCT |
| | CTTGAGAAAACAAAGTACCCGGATTCTGAT |
| | ATCTACTGGAAGAAGCTTGACGACAAGTAC |
| | CATTTCTCATGCCAGTTCACTGCGGATATT |
| | TTCGCAATGAACCACACTGATTTCATCATC |
| | ACTAGTACTTTCCAAGAAATTGCTGGAAGC |
| | AAAGAAACTGTTGGGCAGTATGAAAGCCAC |
| | ACAGCCTTTACTCTTCCCGGATTGTATCGA |
| | GTTGTTCACGGGATTGATGTGTTTGATCCC |
| | AAGTTCAACATTGTCTCTCCTGGTGCTGAT |
| | ATGAGCATCTACTTCCCTTACACAGAGGAG |
| | AAGCGTAGATTGACTAAGTTCCACTCTGAG |
| | ATCGAGGAGCTCCTCTACAGCGATGTTGAG |
| | AACAAAGAGCACTTATGTGTGCTCAAGGAC |
| | AAGAAGAAGCCGATTCTCTTCACAATGGCT |
| | AGGCTTGATCGTGTCAAGAACTTGTCAGGT |
| | CTTGTTGAGTGGTACGGGAAGAACACCCGC |
| | TTGCGTGAGCTAGCTAACTTGGTTGTTGTT |
| | GGAGGAGACAGGAGGAAAGAGTCAAAGGAC |
| | AATGAAGAGAAAGCAGAGATGAAGAAAATG |
| | TATGATCTCATTGAGGAATACAAGCTAAAC |
| | GGTCAGTTCAGGTGGATCTCCTCTCAGATG |
| | GACCGGGTAAGGAACGGTGAGCTGTACCGG |
| | TACATCTGTGACACCAAGGGTGCTTTTGTC |
| | CAACCTGCATTATATGAAGCCTTTGGGTTA |
| | ACTGTTGTGGAGGCTATGACTTGTGGTTTA |
| | CCGACTTTCGCCACTTGCAAAGGTGGTCCA |
| | GCTGAGATCATTGTGCACGGTAAATCGGGT |
| | TTCCACATTGACCCTTACCATGGTGATCAG |
| | GCTGCTGATACTCTTGCTGATTTCTTCACC |
| | AAGTGTAAGGAGGATCCATCTCACTGGGAT |
| | GAGATCTCAAAAGGAGGGCTTCAGAGGATT |
| | GAGGAGAAATACACTTGGCAAATCTATTCA |
| | CAGAGGCTCTTGACATTGACTGGTGTGTAT |
| | GGATTCTGGAAGCATGTCTCGAACCTTGAC |
| | CGTCTTGAGGCTCGCCGTTACCTTGAAATG |
| | TTCTATGCATTGAAGTATCGCCCATTGGCT |
| | CAGGCTGTTCCTCTTGCACAAGATGATTAA |
| | (SEQ ID NO: 26) |
| Beta- | ATGACCCAACTGGATGTGGAGAGCCTGATT |
| glucosidase | CAAGAGCTGACCCTGAACGAAAAGGTGCAA |
| 1 from | CTGCTGAGCGGTAGCGACTTCTGGCATACC |
| Pichia | ACCCCGGTTCGTCGTCTGGGCATCCCGAAG |
| pastoris, | ATGCGTCTGAGCGACGGTCCGAACGGCGTT |
| DNA | CGTGGTACCAAATTCTTTAACGGTGTTCCG |
| | ACCGCGTGCTTCCCGTGCGGTACCGGTCTG |
| | GGCGCGACCTTTGACAAGGAACTGCTGAAA |
| | GAGGCGGGTAGCCTGATGGCGGATGAAGCG |
| | AAAGCGAAAGCGGCGAGCGTGGTTCTGGGT |
| | CCGACCGCGAACATTGCGCGTGGTCCGAAC |
| | GGTGGCCGTGGCTTCGAGAGCTTCGGCGAG |
| | GACCCGGTGGTTAACGGTCTGAGCAGCGCG |
| | GCGATGATCAACGGCCTGCAGGGCAAGTAC |
| | ATTGCGGCGACCATGAAACACTATGTTTGC |
| | AACGATCTGGAAATGGACCGTAACTGCATT |
| | GACGCGCAAGTTAGCCACCGTGCGCTGCGT |
| | GAGGTGTACCTGCTGCCGTTCCAAATCGCG |
| | GTGCGTGATGCGAACCCGCGTGCGATTATG |
| | ACCGCGTATAACAAGGCGAACGGCGAACAC |
| | GTTAGCCAGAGCAAATTCCTGCTGGACGAA |
| | GTGCTGCGTAAGGAGTGGGGCTGGGATGGT |
| | CTGCTGATGAGCGACTGGTTTGGTGTTTAC |
| | GATGCGAAAAGCAGCATCACCAACGGCCTG |
| | GACCTGGAGATGCCGGGTCCGCCGCAGTGC |
| | CGTGTGCACAGCGCGACCGATCACGCGATC |
| | AACAGCGGCGAAATCCACATTAACGATGTT |
| | GACGAGCGTGTGCGTAGCCTGCTGAGCCTG |
| | ATTAACTACTGCCACCAAAGCGGTGTTACC |
| | GAGGAAGATCCGGAAACCAGCGACAACAAC |
| | ACCCCGGAAACCATCGAGAAGCTGCGTAAA |
| | ATCAGCCGTGAGAGCATTGTGCTGCTGAAG |
| | GACGATGACCGTAACCGTAGCATTCTGCCG |
| | CTGAAGAAAAGCGACAAAATCGCGGTTATT |
| | GGTAACAACGCGAAACAAGCGGCGTATTGC |
| | GGTGGCGGTAGCGCGAGCGTGCTGAGCTAT |
| | CACACCACCACCCCGTTCGACAGCATCAAG |
| | AGCCGTCTGGAAGATAGCAACACCCCGGCG |
| | TACACCATTGGTGCGGACGCGTATAAAAAC |
| | CTGCCGCCGCTGGGTCCGCAAATGACCGAT |
| | AGCGACGGCAAGCCGGGTTTTGATGCGAAA |
| | TTCTTTGTTGGCAGCCCGACCAGCAAGGAT |
| | CGTAAACTGATCGACCACTTCCAGCTGACC |
| | AACAGCCAAGTTTTTCTGGTGGACTACTAT |
| | AACGAACAGATCCCGGAAAACAAGGAGTTC |
| | TACGTTGACGTGGAGGGTCAATTTATTCCG |
| | GAGGAAGATGGCACCTATAACTTCGGTCTG |
| | ACCGTGTTTGGTACCGGCCGTCTGTTCGTT |
| | GATGACAAACTGGTTAGCGACAGCAGCCAG |
| | AACCAAACCCCGGGCGATAGCTTCTTTGGT |
| | CTGGCGGCGCAGGAAGTGATCGGCAGCATT |
| | CACCTGGTGAAGGGTAAAGCGTACAAGATC |
| | AAAGTTCTGTATGGCAGCAGCGTGACCCGT |
| | ACCTACGAAATTGCGGCGAGCGTTGCGTTT |
| | GAGGGCGGTGCGTTCACCTTTGGTGCGGCG |
| | AAACAGCGTAACGAAGACGAGGAAATCGCG |
| | CGTGCGGTGGAGATTGCGAAGGCGAACGAC |
| | AAAGTGGTTCTGTGCATCGGCCTGAACCAA |
| | GATTTCGAAAGCGAGGGTTTTGATCGTCCG |
| | GACATCAAGATTCCGGGCGCGACCAACAAA |
| | ATGGTTAGCGCGGTGCTGAAGGCGAACCCG |
| | AACACCGTTATTGTGAACCAGACCGGTACC |
| | CCGGTTGAGATGCCGTGGGCGAGCGATGCG |
| | CCGGTGATCCTGCAAGCGTGGTTTGGCGGT |
| | AGCGAGGCGGGTACCGCGATTGCGGATGTT |
| | CTGTTTGGCGACTACAACCCGAGCGGCAAG |
| | CTGACCGTGACCTTCCCGCTGCGTTTTGAG |
| | GATAACCCGGCGTACCTGAACTTCCAGAGC |
| | AACAAACAAGCGTGCTGGTATGGCGAAGAC |
| | GTTTACGTGGGTTATCGTTACTATGAGACC |
| | ATCGATCGTCCGGTGCTGTTCCCGTTTGGT |
| | CACGGCCTGAGCTTCACCGAGTTCGATTTT |
| | ACCGACATGTTTGTTCGTCTGGAGGAAGAG |
| | AACCTGGAAGTTGAGGTGGTTGTGCGTAAC |
| | ACCGGCAAGTACGACGGTGCGGAAGTGGTG |
| | CAGCTGTATGTTGCGCCGGTTAGCCCGAGC |
| | CTGAAACGTCCGATCAAGGAACTGAAAGAG |
| | TACGCGAAAATTTTCCTGGCGAGCGGTGAA |
| | GCGAAGACCGTTCACCTGAGCGTGCCGATC |
| | AAATACGCGACCAGCTTCTTTGATGAGTAT |
| | CAAAAGAAATGGTGCAGCGAAAAGGGCGAG |
| | TATACCATTCTGCTGGGTAGCAGCAGCGCG |
| | GACATCAAAGTTAGCCAAAGCATCACCCTG |
| | GAAAAAACCACCTTCTGGAAAGGTCTGTAA |
| | (SEQ ID NO: 27) |
| Beta- | ATGAAAAGCCAGCTGATCTTTATGGCTTTG |
| glucosidase | GCCTCCCTTGTAGCAAGTGCACCGCTGGAA |
| 2 from | CACCAGCAGCAGCATCATAAACATGAGAAA |
| Pichia | CGCGCCGTAGTTACGCAGACAGTAACTGTT |
| pastoris, | GCGGCGGGCCAGACAGCAGCAGCGGGTTCC |
| DNA | GCCCAGGCAGTTGTTACCTCAAGCGCGGCG |
| | CCAGCATCCGTTGCTTCAAGTGCGGCCGCG |
| | TCTGCTAGCTCATCTTCTTCCAGCTATACC |
| | TCTGGCGCTTCAGGCGATCTTAGTAGTTTC |
| | AAAGATGGTACTATTAAATGTTCAGAATTC |
| | CCATCAGGGGATGGCGTGGTGTCCGTCTCT |
| | TGGTTAGGCTTCGGCGGCTGGTCTAGTATT |
| | ATGAATCTGCAGGGTGGTACTTCAGAGAGT |
| | TGTGAGAACGGCTATTATTGTTCATATGCA |
| | TGTGAAGCCGGTTATAGCAAAACACAGTGG |
| | CCATCTAACCAGCCGTCAGATGGGAGATCA |
| | GTGGGAGGGTTGCTGTGTAAAGATGGCCTG |
| | TTATATCGCTCCAATACAGCGTTCGATACA |
| | TTATGTGTGCCTGGAAAAGGTACAGCATCC |
| | GTGGAGAATAATGTGTCTAAAGGTATTTCC |
| | ATTTGTAGAACGGATTATCCGGGGTCTGAA |
| | AACATGTGCGTCCCGACGTGGGTCGATGCC |
| | GGTAACTCAAACACCTTGACAGTGGTAGAT |
| | GAAGATAATTATTATGAATGGCAGGGCCTT |
| | AAAACTAGTGCTCAGTATTATGTGAATAAC |
| | GCCGGTGTTAGTGTTGAAGATGGGTGCATC |
| | TGGGGCGATGAGTCCAGCGGCGTTGGAAAC |
| | TGGGCGCCGTTGGTTTTGGGGGCCGGTTCC |
| | ACGGGGGGTCTGACCTATCTGTCTCTGATT |
| | CCGAATCCAAACAACAAAAAAGCACCGAAT |
| | TTTAACGTAAAAATCGTGGCCACGGATGGA |
| | AGTTCAATTAACGGAGATTGCAAATATGAA |
| | AATGGGATCTTTGTCGGTTCTTCAACCGAT |
| | GGCTGCACGGTAACTGTTACCTCAGGTAGT |
| | GCAAAACTGGTTTTTTATTAA |
| | (SEQ ID NO: 28) |
| Beta- | ATGCAGGTTAAATCTATTGTTAATTTACTG |
| glucosidase | CTTGCCTGTTCCTTGGCTGTGGCGCGTCCG |
| 3 from | TTGGAACACGCTCACCATCAGCATGATAAA |
| Pichia | CGCGGCGTTGTAGTAGTAACGAAAACCATC |
| pastoris, | GTCGTTGATGGTAGCACAGTTGAGGCTACC |
| DNA | GCCGCTGCTCAGGTGCAGGAGCATGCAGAA |
| | ACCTTTGCAGAATCAACCCCGTCAGCCGTC |
| | GTTTCCAGTTCATCCGCCCCTTCATCAGCA |
| | AGCTCAGCTTCCGCTCCAGCTAGTTCAGGT |
| | TCTTTTTCAGCTGGTACCAAAGGCGTGACA |
| | TATTCTCCATATCAGGCCGGTGGTGGGTGT |
| | AAAACAGCGGAAGAAGTGGCATCCGATCTG |
| | TCACAGCTTACCGGTTATGAAATTATTCGG |
| | CTTTATGGCGTAGATTGCAACCAGGTTGAG |
| | AACGTGTTTAAAGCCAAAGCCCCTGGCCAG |
| | AAACTTTTTTTGGGTATCTTTTTTGTGGAT |
| | GCCATCGAGTCTGGCGTATCAGCTATCGCA |
| | AGTGCCGTTAAATCCTATGGTTCTTGGGAT |
| | GATGTACACACTGTATCTGTTGGCAACGAG |
| | CTGGTGAACAATGGCGAAGCCACTGTTAGC |
| | CAGATTGGACAGTATGTTAGTACGGCCAAA |
| | TCAGCCTTACGCTCTGCCGGTTTCACAGGG |
| | CCAGTATTGTCTGTTGATACTTTTATTGCA |
| | GTGATTAACAATCCGGGGCTGTGTGATTTC |
| | GCGGATGAATATGTTGCTGTGAACGCCCAT |
| | GCGTTCTTCGATGGGGGTATTGCTGCCTCA |
| | GGGGGGGGCGATTGGGCGGCAGAGCAGATC |
| | CAGCGCGTCTCCAGTGCGTGCGGCGGGAAA |
| | GATGTCTTAATTGTAGAAAGCGGTTGGCCG |
| | TCTAAAGGAGATACGAACGGCGCCGCAGTG |
| | CCGTCAAAATCCAATCAGCAGGCTGCAGTC |
| | CAGAGTCTTGGCCAGAAAATTGGGAGCTCA |
| | TGCATTGCCTTTAACGCATTTAATGATTAT |
| | TGGAAAGCCGATGGTCCGTTCAACGCCGAA |
| | AAATATTGGGGGATCCTTGATAGTTAA |
| | (SEQ ID NO: 29) |
| Beta- | ATGCTGTCCACAATTCTGAATATTTTTATT |
| glucosidase | CTTCTGTTATTCATCCAGGCGTCTCTTCAG |
| 4 from | GCGCCTATTCCGGTGGTGACCAAATATGTG |
| Pichia | ACCGAAGGTATTGCCGTTGTGACTGAAACC |
| pastoris, | AATGTGCGGGTTGTTACTAAAACCATTCCG |
| DNA | ATTGTGCAGGTGCTGATCTCCGATGGTGCA |
| | ACCTATACTCATACCCTGACGACAGTGTCA |
| | ACGGCGGAAGAAAATGGCAACTTCCAGCCT |
| | ATTACCACGACATCTATTGTCAACAAAGAA |
| | GTTGTAGTACCAACAAGCGTAACCCCGAAT |
| | ACCCAGCAGACGCGTCCGACCCAGGTAGAT |
| | ACCACACAGAACAATGCGGATACACCAGCG |
| | GCGCCTACACCATCACCTACTACTAGTTCA |
| | AACAACGGCGTGTTCACCACATATTCCACA |
| | ACACGTAGCGTAGTCACTAGTGTAGTCGTA |
| | GTCGGACCGGATGGAAGCCCTATTGAAAAT |
| | ACTGGACAGACAGCAAACCCTACTACAACT |
| | GCCCCAACTACAAGCACTACTGCTGCCCGG |
| | ACCACAAGCAGTACGTCCACCACACCTACC |
| | GCTAGCTCTACGCCAGGAGGTAATCATCCA |
| | CGTAGCATCGTCTATTCTCCATATTCCGAT |
| | AGCAGTCAGTGTAAAGATGCGACAACGATC |
| | GAAACCGATCTTGAGTTCATTGCCTCTAAA |
| | GGCATCAGCGCGGTACGTATTTATGGCAAT |
| | GATTGTAACTATCTTACAGTTGTTTTGCCT |
| | AAATGTGCCAGTCTGGGATTAAAAGTGAAT |
| | CAGGGCTTTTGGATTGGTCCAAGTGGAGTA |
| | GATAGCATCGATGATGCAGTACAGGAGTTT |
| | ATTCAGGCAGTCAACGGCAACAACGGCTTT |
| | AATTGGGATTTATTCGAATTAATTACCGTC |
| | GGAAACGAAGCAATCAGTGCCGGTTATGTT |
| | TCAGCGAGCTCCCTGATTTCCAAAATTAAA |
| | GAAGTATCTAGCATTCTGAGCTCCGCGGGT |
| | TATACTGGTCCAATTACCACAGCCGAACCG |
| | CCTAACGTATATGAGGATTATGGCGATCTG |
| | TGCTCAACCGATGTAATGTCCATCGTGGGT |
| | GTAAACGCGCATTCCTATTTTAATACCCTT |
| | TTTGCGGCCTCCGATTCAGGTTCATTTGTG |
| | AAATCACAGATCGAAGTAGTCCAGAAAGCA |
| | TGCTCACGTTCCGATATTACTATTATTGAA |
| | ACCGGGTATCCGTCCCAGGGAGCTACCAAT |
| | GGAAAAAACGTTCCTAGTAAAGAGAATCAG |
| | AAAACAGCGATTTTTTCAATCTTTGAGGTC |
| | GTTGGAACAGATGTAACTATTCTTAGTACT |
| | TATGATGATTTGTGGAAAGATCCTGGACCG |
| | TATGGGATTGAACAGTTTTTTGGTGCGATC |
| | GATCTTTTTTCTTAA |
| | (SEQ ID NO: 30) |

### Rebaudioside A

Rebaudioside A is a steviol glycoside produced in Stevia plants. Rebaudioside A has the molecular formula C₄₄H₇₀O₂₃ and the IUPAC name, 13-[(2-O-β-D-glucopyranosyl -3-O-β-D-glucopyranosyl-β-D-glucopyranosyl)oxy]-ent-kaur-16-en-19-oic acid β-D -glucopyranosyl ester.

Rebaudioside A may be purified from Stevia leaf extracts, or recombinantly or synthetically produced. In some embodiments, rebaudioside A is produced via covalently coupling a glucose to stevioside by UGT76G1 or UGT76G1-SUS fusion enzyme. In some embodiments, rebaudioside A is produced from a reaction mixture comprising stevioside, substrates selected from the group consisting of sucrose, uridine diphosphate (UDP), uridine diphosphate-glucose (UDP-glucose), and combinations thereof, and UGT76G1 (e.g., SEQ ID NO: 1) or UGT76G1-SUS fusion enzyme (e.g., SEQ ID NO: 9), with or without additional sucrose synthase (e.g., SEQ ID NO: 8).

In some embodiments, rebaudioside A is produced via covalently coupling a glucose to rebaudioside D by EUGT11, HV1, EUGT11-SUS fusion enzyme, or HV1-SUS fusion enzyme. In some embodiments, rebaudioside A is produced from a reaction mixture comprising rebaudioside D, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

In some embodiments, rebaudioside A is produced via removing a glucosyl group from Reb I (at position C19) or Reb D (at position C13) by a beta-glucosidase. In some embodiments, rebaudioside A is produced from a reaction mixture comprising Reb I or Reb D and a beta glucosidase.

### Rebaudioside D

Rebaudioside D has the molecular formula C₅₀H₈₀O₂₈ and the IUPAC name, [4,5-dihydroxy-6-(hydroxymethyl)-3-[3,4,5-trihydroxy-6-(hydroxymethypoxan-2-yl]oxyoxan -2-yl]13-[5-hydroxy-6-(hydroxymethyl)-3,4-bis[[3,4,5-trihydroxy-6-(hydroxymethyl)oxan-2-yl]oxy]oxan-2-yl]oxy-5,9-dimethyl-14-methylidenetetracyclo[11.2.1.0^{1,10}.0^{4,9}]hexadecane-5-carboxylate.

In some embodiments, rebaudioside D is produced via covalently coupling a glucose to rebaudioside E UGT76G1 or aa UTG76G1-SUS fusion enzyme. In some embodiments, rebaudioside D is produced from a reaction mixture comprising rebaudioside E, substrates selected from the group consisting of sucrose, uridine diphosphate (UDP), uridine diphosphate-glucose (UDP-glucose), and combinations thereof, and UGT76G1 (e.g., SEQ ID NO: 1) or UGT76G1-SUS fusion enzyme (e.g., SEQ ID NO: 9), with or without additional sucrose synthase (e.g., SEQ ID NO: 8).

In some embodiments, rebaudioside D is produced via covalently coupling a glucose to rebaudioside A by EUGT11, HV1, EUGT11-SUS fusion enzyme, or HV1-SUS fusion enzyme. In some embodiments, rebaudioside D is produced from a reaction mixture comprising rebaudioside A, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

In some embodiments, rebaudioside D is produced via covalently coupling two glucoses to stevioside. For example, a glucose is covalently coupled to the stevioside to produce rebaudioside A and/or rebaudioside E. A glucose can then be covalently coupled to the rebaudioside A and/or rebaudioside E to produce rebaudioside D. In some embodiments, rebaudioside D is produced by a reaction mixture comprising stevioside; a substrate selected from the group consisting of sucrose, uridine diphosphate (UDP), uridine diphosphate-glucose (UDP-glucose), and combinations thereof; and a combination of UGT76G1 (e.g., SEQ ID NO: 1) or UGT76G1-SUS fusion enzyme (e.g., SEQ ID NO: 9) and HV1 (e.g., SEQ ID NO: 7) or HV1-SUS fusion enzyme (e.g., SEQ ID NO: 11), with or without additional sucrose synthase (e.g., SEQ ID NO: 8).

### Rebaudioside E

Rebaudioside E is a steviol glycoside produced in Stevia plants. Rebaudioside E has the molecular formula C₄₄H₇₀O₂₃ and the IUPAC name, [(2S,3R,4S,5S,6R)-4,5-dihydroxy -6-(hydroxymethyl)-3-[(2S,3R,4S,5S,6R)-3,4,5-trihydroxy-6-(hydroxymethyl)oxan-2-yl]oxyoxan-2-yl] (1R,4S,5R,9S,10R,13S)-13-[(2S,3R,4S,5S,6R)-4,5-dihydroxy-6-(hydroxymethyl)-3-[(2S,3R,4S,5S,6R)-3,4,5-trihydroxy-6-(hydroxymethyl)oxan-2-yl]oxyoxan -2-yl]oxy-5,9-dimethyl-14-methylidenetetracyclo[11.2.1.0^{1,10}.0^{4,9}]hexadecane-5-carboxylate.

Rebaudioside E may be purified from Stevia leaf extracts, or recombinantly or synthetically produced. In some embodiments, rebaudioside E is produced via covalently coupling one or more glucoses to stevioside, rubusoside, or rebaudioside KA by an UDP-glycosyltransferase selected from the group consisting of HV1, EUGT11, UGT76G1, a HV1-SUS fusion enzyme, a EUGT11-SUS fusion enzyme, and a UTG76G1-SUS fusion enzyme.

In some embodiments, rebaudioside E is produced via covalently coupling a glucose to rebaudioside KA by the HV1, EUGT11, HV1-SUS fusion enzyme, or EUGT11-SUS fusion enzyme. In some embodiments, rebaudioside E is produced from a reaction mixture comprising rebaudioside KA, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

In some embodiments, rebaudioside E is produced via covalently coupling a glucose to stevioside by the HV1, EUGT11, HV1-SUS fusion enzyme, or EUGT11-SUS fusion enzyme. In some embodiments, rebaudioside E is produced from a reaction mixture comprising stevioside, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

In some embodiments, rebaudioside E is produced via covalently coupling two glucoses to rubusoside by the HV1, EUGT11, HV1-SUS fusion enzyme, or EUGT11-SUS fusion enzyme. For example, a glucose is covalently coupled to the rubusoside to produce rebaudioside KA. A glucose can then be covalently coupled to the rebaudioside KA to produce rebaudioside E. In some embodiments, rebaudioside E is produced from a reaction mixture comprising rubusoside, substrates selected from the group consisting of sucrose, uridine diphosphate (UDP), uridine diphosphate-glucose (UDP-glucose), and combinations thereof, and EUGT11 (e.g., SEQ ID NO: 5), HV1 (e.g., SEQ ID NO: 7), EUGT11-SUS fusion enzyme (e.g., SEQ ID NO: 10), or HV1-SUS fusion enzyme (e.g., SEQ ID NO: 11), with or without additional sucrose synthase (e.g., SEQ ID NO: 8).

### Rebaudioside M

Rebaudioside M has the molecular formula C₅₆H₉₀O₃₃ and the IUPAC name, 13-[(2-O-β-D-glucopyranosyl-3-O-β-D-glucopyranosyl-β-D-glucopyranosyl)oxy] ent-kaur-16-en -19-oic acid-[(2-O-β-D-glucopyranosyl-3-O-β-D-glucopyranosyl-β-D-glucopyranosyl)ester.

In some embodiments, rebaudioside M is produced via covalently coupling one or more glucoses to stevioside, rebaudioside A, rebaudioside E, or rebaudioside D by an UDP-glycosyltransferase selected from the group consisting of HV1, UGT76G1, a HV1-SUS fusion enzyme, and a UTG76G1-SUS fusion enzyme.

In some embodiments, rebaudioside M is produced via covalently coupling a glucose to rebaudioside D by the UGT76G1 or UGT76G1-SUS fusion enzyme. In some embodiments, rebaudioside M is produced from a reaction mixture comprising rebaudioside D, substrates selected from the group consisting of sucrose, uridine diphosphate (UDP), uridine diphosphate-glucose (UDP-glucose), and combinations thereof, and UGT76G1 (e.g., SEQ ID No: 1) or UGT76G1-SUS fusion enzyme (e.g., SEQ ID NO: 9), with or without additional sucrose synthase (e.g., SEQ ID NO: 8).

For example, a glucose is covalently coupled to the stevioside to produce rebaudioside A and/or rebaudioside E. A glucose can then be covalently coupled to the rebaudioside A and/or rebaudioside E to produce rebaudioside D, and a glucose can then be covalently coupled to the rebaudioside D to produce rebaudioside M.

### Rebaudioside I

Rebaudioside I has the molecular formula C₅₀H₈₀O₂₈ and the IUPAC name, 13-[(2-O-β-D-glucopyranosyl-3-O-β-D-glucopyranosyl)-β-D-glucopyranosypoxy]-ent-kaur-16-en-19oic acid-(3-O-β-D-glucopyranosyl)-β-D-glucopyranosyl), ester.

In some embodiments, rebaudioside I is produced via covalently coupling a glucose to a steviol glycoside (e.g., rebaudioside A) by an UGT76G1, a UTG76G1-SUS fusion enzyme, or UGT76G1 variants such as UGT76G1 CP1, UGT76G1 CP2, and UGT76G1 L200A. In some embodiments, rebaudioside I produced by a reaction mixture comprising a steviol glycoside (e.g., rebaudioside A); a substrate selected from the group consisting of sucrose, uridine diphosphate (UDP), and uridine diphosphate-glucose (UDP-glucose); and UGT76G1 (e.g., SEQ ID No: 1), UGT76G1-SUS fusion enzyme (e.g., SEQ ID NO: 9), UTG76G1 CP1 variant (e.g., SEQ ID NO: 3), UTG76G1 CP2 variant (e.g., SEQ ID NO: 4), or UTG76G1 L200A variant (e.g., SEQ ID NO: 2), with or without additional sucrose synthase (e.g., SEQ ID NO: 8).

### Steviol Glycoside Formulations

Provided herein, in some aspects, are steviol glycoside formulations containing a combination of rebaudiosides that provide a taste profile similar to sugar throughout the entire taste profile, from onset of sweetness to sweetness linger, as determined through, e.g., the use of a panel of tasters, by means of a sensory evaluation, as well as evaluation of its physical characteristics and capacity to replace a food/feed stuff made with a full or normal complement of sucrose.

Where initial sensory testing of some blends in non-carbonated beverages detected slightly less sweet than full sugar product and some bitterness in aftertaste, the steviol glycoside formulations described herein were re-balanced to increase sweetness and reduce bitterness.

Measuring the perceived sweetness of a solution is typically done by calculating its sucrose equivalence. The sucrose equivalence value is the standard used to measure sweetness as compared to the baseline standard of sucrose—table sugar. All sweeteners, including sugarless and high intensity sweeteners, are measured against sucrose. Sucrose equivalence may be defined as the amount of sweetener required to impart the comparable or equivalent level of sweetness perceived from a given amount of sucrose. One method of measuring the perceived sweetness of a solution is to match it with a stock sucrose solution of known concentration. For example, the blend of interest is added at a predetermined concentration to a water solution. A number of expert panel members then taste the solution and compare it to a battery of stock sucrose solutions ranging from 0.5% to 10% at increments of 0.5%. Each panel member decides which sucrose solution is equally sweet in comparison to the solution containing the test blend. The formulations provided herein were designed to provide a taste sensation equivalent to those same food products using sucrose.

In some embodiments, the steviol glycoside formulation comprises rebaudioside A, rebaudioside D, rebaudioside E, and rebaudioside M. In some embodiments, the steviol glycoside formulation comprises rebaudioside A, rebaudioside D, rebaudioside E, rebaudioside M, and rebaudioside I. In some embodiments, the steviol glycoside formulation consists essentially of rebaudioside A, rebaudioside D, rebaudioside E, and rebaudioside M. In some embodiments, the steviol glycoside formulation consists essentially of rebaudioside A, rebaudioside D, rebaudioside E, rebaudioside M, and rebaudioside I.

In some embodiments, the steviol glycoside formulation consists essentially of about 40-60 wt. % rebaudioside A (e.g., about 40 wt. %, about 45 wt. %, about 50 wt. %, about 55 wt. %, or about 60 wt. %), about 15-30 wt. % rebaudioside E (e.g., about 15 wt. %, about 20 wt. %, about 25 wt. %, or about 30 wt. %), about 10-17 wt. % rebaudioside D (e.g., about 10 wt. %, about 15 wt. %, or about 17 wt. %), and/or about 5-10 wt. % rebaudioside M (e.g., about 5 wt. %, about 8 wt. %, or about 10 wt. %). “wt. %” means the % of the weight of the particular anhydrous rebaudioside of the weight of all anhydrous rebaudiosides in the formulation.

In some embodiments, the steviol glycoside formulation described herein consists essentially of rebaudioside A, rebaudioside D, rebaudioside E, and rebaudioside M, and comprises about 40-60 wt. %, 40-55 wt. %, 40-50 wt. %, 40-45 wt. %, 46-60 wt. %, 45-55 wt. %, 45-50 wt. %, 50-60 wt. %, 50-55 wt. %, or 55-60 wt. %, of rebaudioside A. In some embodiments, the steviol glycoside formulation described herein consists essentially of rebaudioside A, rebaudioside D, rebaudioside E, and rebaudioside M and comprises about 15-30 wt. %, 15-25 wt. %, 15-20 wt. %, 20-30 wt. %, 20-25 wt. %, or 25-30 wt. % of rebaudioside E. In some embodiments, the steviol glycoside formulation described herein consists essentially of rebaudioside A, rebaudioside D, rebaudioside E, and rebaudioside M and comprises 10-17 wt. %, 10-15 wt. %, or 15-17 wt. % of rebaudioside D. In some embodiments, the steviol glycoside formulation described herein consists essentially of rebaudioside A, rebaudioside D, rebaudioside E, and rebaudioside M and comprises 5-10 wt. %, 5-8 wt. %, or 8-10 wt. % of rebaudioside M. In some embodiments, the steviol glycoside formulation described herein comprises 5-10 wt. %, 5-8 wt. %, or 8-10 wt. % of rebaudioside M. In some embodiments, the steviol glycoside formulation described herein consists essentially of about 58.33 wt. % of rebaudioside A, about 8.33 wt. % of rebaudioside M, about 16.67 wt. % of rebaudioside D, and about 16.67 wt. % of rebaudioside E.

In some embodiments, the steviol glycoside formulation consists essentially of about 40-60 wt. % rebaudioside A (e.g., about 40 wt. %, about 45 wt. %, about 50 wt. %, about 55 wt. %, or about 60 wt. %), about 15-30 wt. % rebaudioside E (e.g., about 15 wt. %, about 20 wt. %, about 25 wt. %, or about 30 wt. %), about 10-17 wt. % rebaudioside D (e.g., about 10 wt. %, about 15 wt. %, or about 17 wt. %), about 5-10 wt. % rebaudioside M (e.g., about 5 wt. %, about 8 wt. %, or about 10 wt. %), and/or about 2-8 wt. % rebaudioside I. In some embodiments, the steviol glycoside formulation described herein consists essentially of rebaudioside A, rebaudioside D, rebaudioside E, and rebaudioside M and comprises about 40-60 wt. %, 40-55 wt. %, 40-50 wt. %, 40-45 wt. %, 46-60 wt. %, 45-55 wt. %, 45-50 wt. %, 50-60 wt. %, 50-55 wt. %, or 55-60 wt. %, of rebaudioside A. In some embodiments, the steviol glycoside formulation described herein consists essentially of rebaudioside A, rebaudioside D, rebaudioside E, and rebaudioside M and comprises about 15-30 wt. %, 15-25 wt. %, 15-20 wt. %, 20-30 wt. %, 20-25 wt. %, or 25-30 wt. % of rebaudioside E. In some embodiments, the steviol glycoside formulation described herein consists essentially of rebaudioside A, rebaudioside D, rebaudioside E, and rebaudioside M and comprises about 10-17 wt. %, 10-15 wt. %, or 15-17 wt. % of rebaudioside D. In some embodiments, the steviol glycoside formulation described herein consists essentially of rebaudioside A, rebaudioside D, rebaudioside E, and rebaudioside M and comprises about 5-10 wt. %, 5-8 wt. %, or 8-10 wt. % of rebaudioside M. In some embodiments, the steviol glycoside formulation described herein consists essentially of rebaudioside A, rebaudioside D, rebaudioside E, and rebaudioside M and comprises about 2-8 wt. %, 5-8 wt. %, or 2-5 wt. % of rebaudioside I. In some embodiments, the steviol glycoside formulation described herein consists essentially of about 54.69 wt. % of rebaudioside A, about 7.04 wt. % of rebaudioside M, about 14.66 wt. % of rebaudioside D, about 19.21 wt. % of rebaudioside E, and about 4.4 wt. % of rebaudioside I.

In some embodiments, the steviol glycoside formulation consists essentially of rebaudioside A, rebaudioside D, rebaudioside E, and rebaudioside M, wherein rebaudioside A is present in a concentration of about 300-600 ppm (e.g., about 300 ppm, about 350 ppm, about 400 ppm, about 450 ppm, about 500 ppm, about 550 ppm, or about 600 ppm), rebaudioside E is present in a concentration of about 50-200 ppm (e.g., about 50 ppm, about 100 pm, about 150 ppm, or about 200 ppm), rebaudioside D is present in a concentration of about 50-200 ppm (e.g., about 50 ppm, about 100 pm, about 150 ppm, or about 200 ppm), and/or rebaudioside M is present in a concentration of about 200-500 ppm (e.g., about 200 ppm, about 250 ppm, about 300 ppm, about 350 ppm, about 400 ppm, about 450 ppm, or about 500 ppm). In some embodiments, the steviol glycoside formulation consists essentially of rebaudioside A, rebaudioside D, rebaudioside E, and rebaudioside M, wherein rebaudioside A is present in a concentration of about 300-600 ppm, 300-550 ppm, 300-500 ppm, 300-450 ppm, 300-400 ppm, 300-350 ppm, 350-600 ppm, 350-550 ppm, 350-500 ppm, 350-450 ppm, 350-400 ppm, 400-600 ppm, 400-550 ppm, 400-500 ppm, 400-450 ppm, 450-600 ppm, 450-550 ppm, 450-500 ppm, 500-600 ppm, 500-550 ppm, or 550-600 ppm. In some embodiments, the steviol glycoside formulation consists essentially of rebaudioside A, rebaudioside D, rebaudioside E, and rebaudioside M, wherein rebaudioside E is present in a concentration of about 50-200 ppm, 50-150 ppm, 50-100 ppm, 100-200 ppm, 100-150 ppm, or 150-200 ppm. In some embodiments, the steviol glycoside formulation consists essentially of rebaudioside A, rebaudioside D, rebaudioside E, and rebaudioside M, wherein rebaudioside D is present in a concentration of about 50-200 ppm, 50-150 ppm, 50-100 ppm, 100-200 ppm, 100-150 ppm, or 150-200 ppm. In some embodiments, the steviol glycoside formulation consists essentially of rebaudioside A, rebaudioside D, rebaudioside E, and rebaudioside M, wherein rebaudioside M is present in a concentration of about 200-500 ppm, 200-450 ppm, 200-400 ppm, 200-350 ppm, 200-300 ppm, 200-250 ppm, 250-500 ppm, 250-450 ppm, 250-400 ppm, 250-350 ppm, 250-300 ppm, 300-500 ppm, 300-450 ppm, 300-400 ppm, 300-350 ppm, 350-500 ppm, 350-400 ppm, 400-500 ppm, 400-450 ppm, or 450-500 ppm. In some embodiments, the steviol glycoside formulation consists essentially of rebaudioside A, rebaudioside D, rebaudioside E, and rebaudioside M, wherein rebaudioside A is present in a concentration of about 500 ppm, rebaudioside M is present in a concentration of about 350 ppm, rebaudioside D is present in a concentration of about 100 ppm, and rebaudioside E is present in a concentration of about 100 ppm.

In some embodiments, the steviol glycoside formulation consists essentially of rebaudioside A, rebaudioside D, rebaudioside E, and rebaudioside M, wherein rebaudioside A is present in a concentration of about 200-500 ppm (e.g., about 200 ppm, about 250 ppm, about 300 ppm, about 350 ppm, about 400 ppm, about 450 ppm, or about 500 ppm), rebaudioside E is present in a concentration of about 50-300 ppm (e.g., about 50 ppm, about 100 pm, about 150 ppm, about 200 ppm, about 250 ppm, or about 300 ppm), rebaudioside D is present in a concentration of about 50-300 ppm (e.g., about 50 ppm, about 100 pm, about 150 ppm, about 200 ppm, about 250 ppm, or about 300 ppm), rebaudioside M is present in a concentration of about 5-100 ppm (e.g., about 5 ppm, about 50 ppm, or about 100 ppm), and/or rebaudioside I is present in a concentration of about 5-50 ppm (e.g., about 5 ppm or about 50 ppm). In some embodiments, the steviol glycoside formulation consists essentially of rebaudioside A, rebaudioside D, rebaudioside E, and rebaudioside M, wherein rebaudioside A is present in a concentration of about 200-500 ppm, 200-450 ppm, 200-400 ppm, 200-350 ppm, 200-300 ppm, 200-250 ppm, 250-500 ppm, 250-450 ppm, 250-400 ppm, 250-350 ppm, 250-300 ppm, 300-500 ppm, 300-450 ppm, 300-400 ppm, 300-350 ppm, 350-500 ppm, 350-400 ppm, 400-500 ppm, 400-450 ppm, or 450-500 ppm. In some embodiments, the steviol glycoside formulation consists essentially of rebaudioside A, rebaudioside D, rebaudioside E, and rebaudioside M, wherein rebaudioside E is present in a concentration of about 50-300 ppm, 50-250 ppm, 50-150 ppm, 50-100 ppm, 100-300 ppm, 100-250 ppm, 100-200 ppm, 100-150 ppm, 150-300 ppm, 150-250 ppm, 150-200 ppm, 200-300 ppm, 200-250 ppm, or 250-300 ppm. In some embodiments, the steviol glycoside formulation consists essentially of rebaudioside A, rebaudioside D, rebaudioside E, and rebaudioside M, wherein rebaudioside D is present in a concentration of about 50-300 ppm, 50-250 ppm, 50-150 ppm, 50-100 ppm, 100-300 ppm, 100-250 ppm, 100-200 ppm, 100-150 ppm, 150-300 ppm, 150-250 ppm, 150-200 ppm, 200-300 ppm, 200-250 ppm, or 250-300 ppm. In some embodiments, the steviol glycoside formulation consists essentially of rebaudioside A, rebaudioside D, rebaudioside E, and rebaudioside M, wherein rebaudioside M is present in a concentration of about 5-100 ppm, 1-50 ppm, or 50-100 ppm. In some embodiments, the steviol glycoside formulation consists essentially of rebaudioside A, rebaudioside D, rebaudioside E, and rebaudioside M, wherein rebaudioside I is present in a concentration of about 5-50 ppm, 5-25 ppm, or 25-50 ppm. In some embodiments, the steviol glycoside formulation consists essentially of rebaudioside A, rebaudioside D, rebaudioside E, and rebaudioside M, wherein rebaudioside A is present in a concentration of about 373 ppm, rebaudioside M is present in a concentration of about 48 ppm, rebaudioside D is present in a concentration of about 100 ppm, rebaudioside E is present in a concentration of about 131 ppm, and rebaudioside I is present in a concentration of about 30 ppm.

In some embodiments, the steviol glycoside formulation consisting essentially of rebaudioside A, rebaudioside E, rebaudioside D and rebaudioside M, wherein Reb A is present in an amount of about 300-600 ppm (e.g., about 300 ppm, about 350 ppm, about 400 ppm, about 450 ppm, about 500 ppm, about 550 ppm, or about 600 ppm); Reb E is present in an amount of from about 50-250 ppm (e.g., about 50 ppm, about 100 pm, about 150 ppm, about 200 ppm, or about 250 ppm); Reb D is present in an amount of about 10-200 ppm (e.g., about 10 ppm, about 50 ppm, about 100 pm, about 150 ppm, or about 200 ppm); and/or Reb M is present in an amount of about 10-150 ppm (e.g., about 10 ppm, about 50 ppm, about 100 pm, or about 150 ppm). In some embodiments, the steviol glycoside formulation consisting essentially of rebaudioside A, rebaudioside E, rebaudioside D, rebaudioside M, and rebaudioside I, wherein Reb A is present in an amount of about 300-600 ppm (e.g., about 300 ppm, about 350 ppm, about 400 ppm, about 450 ppm, about 500 ppm, about 550 ppm, or about 600 ppm); Reb E is present in an amount of from about 50-250 ppm (e.g., about 50 ppm, about 100 pm, about 150 ppm, about 200 ppm, or about 250 ppm); Reb D is present in an amount of about 10-200 ppm (e.g., about 10 ppm, about 50 ppm, about 100 pm, about 150 ppm, or about 200 ppm); Reb M is present in an amount of about 10-150 ppm (e.g., about 10 ppm, about 50 ppm, about 100 ppm, or about 150 ppm); and/or Reb I is present in an amount of about 1-50 ppm (e.g., about 1 ppm, about 10 ppm, about 25 ppm, or about 50 ppm).

### Orally Consumable Products

The steviol glycoside formulations described herein provide more consistent and more stable low or non-caloric sweetening compositions not previously available for food or feed manufacturers. The use of the steviol glycoside formulations described herein have been found to maintain the sensory qualities, the shelf-life and the solubility profile of orally consumable products. Any one of the steviol glycoside formulations described herein can be used for the production of baked goods, dairy products, spreads, margarines, sports products, nutrition bars and infant formulas, feed, aquaculture, nutraceuticals and medicinal products. In each, the enhanced nutritional content or off-flavors can be masked with the steviol glycoside formulations described herein.

In some embodiments, any one of the steviol glycoside formulations described herein may be used for creating or enhancing a sweetening effect of an orally consumable products. In some embodiments, methods of creating or enhancing a sweetening effect of an orally consumable product comprises adding an amount of any one of the steviol glycoside formulations described herein sufficient to produce the desired degree of sweetness to the orally consumable product.

Accordingly, other aspects of the present disclosure provide orally consumable products comprising any one of the steviol glycoside formulations described herein. In some embodiments, the orally consumable product is selected from the group consisting of a food composition, a beverage product, a dietary supplement, a nutraceutical, an edible gel mix, an edible gel composition, a pharmaceutical composition, a dental and oral hygiene composition, and an animal feed.

In some embodiments, the orally consumable product comprising any one of the steviol glycoside formulations described herein is a dental and oral hygiene composition. Examples of suitable dental and oral hygiene compositions can be, for example, toothpastes, tooth polishes, dental floss, mouthwashes, mouth rinses, dentrifices, mouth sprays, mouth refreshers, plaque rinses, dental pain relievers, and the like. In some embodiments, the dental and oral hygiene composition is a toothpaste. In some embodiments, in a dental and oral hygiene composition, the steviol glycoside formulation is present in a concentration of about 50-800 ppm (e.g., about 50 ppm, about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, or about 800 ppm). In some embodiments, in a dental and oral hygiene composition, the steviol glycoside formulation is present in the range of about 0.0003% to about 1.0% (e.g., about 0.0003%, about 0.0005%, about 0.001%, about 0.005%, about 0.1%, about 0.2%, about 0.3%, about 0.4%, about 0.5%, about 0.6%, about 0.7%, about 0.8%, about 0.9%, or about 1.0%) by weight of the total weight of the dental and oral hygiene composition.

In some embodiments, the orally consumable product comprising any one of the steviol glycoside formulations described herein is a pharmaceutical composition. In some embodiments, the pharmaceutical composition comprises any one of the steviol glycoside formulations described herein, and further comprises one or more pharmaceutically acceptable excipients. In some embodiments, pharmaceutical compositions of the present disclosure can be used to formulate pharmaceutical drugs containing one or more active agents that exert a biological effect. Accordingly, in some embodiments, pharmaceutical compositions of the present disclosure can contain one or more active agents that exert a biological effect. Suitable active agents are well known in the art (e.g., The Physician's Desk Reference). Such compositions can be prepared according to procedures well known in the art, for example, as described in Remington's Pharmaceutical Sciences, Mack Publishing Co., Easton, Pa., USA.

In some embodiments, in a pharmaceutical composition, the steviol glycoside formulation is present in a concentration of about 50-800 ppm (e.g., about 50 ppm, about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, or about 800 ppm). In some embodiments, in a pharmaceutical composition, the steviol glycoside formulation is present in the range of about 0.0004% to about 1.25% (e.g., about 0.0004%, about 0.0005%, about 0.001%, about 0.005%, about 0.1%, about 0.2%, about 0.3%, about 0.4%, about 0.5%, about 0.6%, about 0.7%, about 0.8%, about 0.9%, about 1.0%, about 1.1%, about 1.2%, or about 1.25%) by weight of the total weight of the pharmaceutical composition.

In some embodiments, the orally consumable product comprising any one of the steviol glycoside formulations described herein is a beverage (e.g., a carbonated beverage product or a non-carbonated beverage product). The beverage can also be, for example, a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, or an energy drink, isotonic drinks, low-calorie drinks, zero-calorie drinks, vegetable juices, juice drinks, dairy drinks, yoghurt drinks, alcohol beverages, and powdered beverages.

In some embodiments, the beverage of the present disclosure comprises any one of the steviol glycoside formulations described herein, and further comprises one or more beverage ingredients such as, for example, acidulants, fruit juices and/or vegetable juices, pulp, etc., flavorings, coloring, preservatives, vitamins, minerals, electrolytes, erythritol, tagatose, glycerine, and carbon dioxide. The beverages described herein may be provided in any suitable form, such as a beverage concentrate and a carbonated, ready-to-drink beverage.

In certain embodiments, the beverages of the present disclosure can have any of numerous different specific formulations or constitutions. The formulation of a beverage of the present disclosure can vary to a certain extent, depending upon such factors as the product's intended market segment, its desired nutritional characteristics, flavor profile, and the like. For example, in certain embodiments, it can generally be an option to add further ingredients to the formulation of a particular beverage product. For example, additional (i.e., more and/or other) sweeteners can be added, flavorings, electrolytes, vitamins, fruit juices or other fruit products, tastents, masking agents and the like, flavor enhancers, and/or carbonation typically may be added to any such formulations to vary the taste, mouthfeel, nutritional characteristics, etc.

In some embodiments, in a beverage, the steviol glycoside formulation is present in a concentration of about 65-800 ppm (e.g., about 65 ppm, about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, or about 800 ppm). In some embodiments, in a beverage, the steviol glycoside formulation is present in the range of about 0.0005% to about 1% (e.g., about 0.0005%, about 0.001%, about 0.005%, about 0.1%, about 0.2%, about 0.3%, about 0.4%, about 0.5%, about 0.6%, about 0.7%, about 0.8%, about 0.9%, or about 1.0%) by weight of the total weight of the beverage.

In some embodiments, the orally consumable product comprising any one of the steviol glycoside formulations described herein is a food composition. A “food composition” refers to any solid or liquid ingestible material that can, but need not, have a nutritional value and be intended for consumption by humans and animals. Examples of suitable food product compositions can be, for example, confectionary compositions, such as candies, mints, fruit flavored drops, cocoa products, chocolates, and the like; condiments, such as ketchup, mustard, mayonnaise, and the like; chewing gums; cereal compositions; baked goods, such as breads, cakes, pies, cookies, and the like; dairy products, such as milk, cheese, cream, ice cream, sour cream, yogurt, sherbet, and the like; tabletop sweetener compositions; soups; stews; convenience foods; meats, such as ham, bacon, sausages, jerky, and the like; gelatins and gelatin-like products such as jams, jellies, preserves, and the like; fruits; vegetables; egg products; icings; syrups including molasses; snacks; nut meats and nut products; and animal feed. Other non-limiting examples of food compositions include bakery products, cookies, biscuits, baking mixes, cereals, confectioneries, candies, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables, herbs, spices and seasonings, natural and synthetic flavors, and flavor enhancers, such as monosodium glutamate, prepared packaged products, such as dietetic sweeteners, liquid sweeteners, granulated flavor mixes, pet foods, livestock feed, tobacco, and materials for baking applications, such as powdered baking mixes for the preparation of breads, cookies, cakes, pancakes, donuts and the like.

In some embodiments, the food composition is selected from the group consisting of spreads, margarines, sports products, nutrition bars, infant formulas, mayonnaise, confectionary composition, a condiment, a chewing gum, a cereal composition, a baked good, a dairy product, and a tabletop sweetener composition. In some embodiments, the food composition is a food composition included in Table 3. In some embodiments, the food composition is a yogurt. In some embodiments, the food composition is frozen. In some embodiments, the food composition is ice cream.

TABLE 3
Examples of food compositions

| | DAIRY | | PREPARED | OIL BASED | SNACK |
|---|---|---|---|---|---|
| BEVERAGES | PRODUCTS | BAKING | FOODS | PRODUCTS | FOODS |
| Soy milks | Cheeses | Breads | Entrees | Salad | Granola |
| Smoothies | Cream | Rolls | Side Dishes | Dressing | Cereals |
| Fruit Juices | Cheeses | Cakes | Soups | Mayonnaise | Snack/ |
| Dairy Drinks | Sour Cream | Pastries | Sauces | Margarine/ | Nutritional |
| | Yogurt | Cookies | Processed | Spreads | Bars |
| | Yogurt | Crackers | Meats | Shortening | Confectionary |
| | Drinks | Muffins | Processed | | |
| | Non-Dairy | | Fish | | |
| | Creamers | | Pet Foods | | |
| | Dips | | | | |

Food compositions described herein include any preparations or compositions which are suitable for consumption and are used for nutrition or enjoyment purposes. They are generally products which are intended to be eaten by humans or animals and introduced into the body through the mouth, to remain there for a certain time and then either be eaten (e.g. ready-to-eat foodstuffs or feeds, see also herein below) or removed (e.g. chewing gums). Such products include any substances or products which in the processed, partially processed or unprocessed state are to be ingested by humans or animals. They also include substances which are added to orally consumable products during their manufacture, preparation or treatment and which are intended to be introduced into the human or animal oral cavity.

The food compositions according to the disclosure also include substances which in the unchanged, treated or prepared state are to be swallowed by a human or animal and then digested; in this respect, the orally consumable products according to the disclosure also include casings, coatings or other encapsulations which are to be swallowed at the same time or which may be expected to be swallowed. The expression “food composition” covers ready-to-eat foodstuffs, beverages and feeds, that is to say foodstuffs, beverages or feeds that are already complete in terms of the substances that are important for the taste. The expressions “ready-to-eat foodstuff” and “ready-to-eat feed” also include drinks as well as solid or semi-solid ready-to-eat foodstuffs or feeds. Examples which may be mentioned are frozen products, which must be thawed and heated to eating temperature before they are eaten. Products such as yoghurt or ice-cream as well as chewing gums or hard caramels are also included among the ready-to-eat foodstuffs or feeds of the current disclosure. SAME WITH THE ABOVE TWO PARAGRAPHS.

In some embodiments, in a food composition, the steviol glycoside formulation is present in a concentration of about 50-700 ppm (e.g., about 50 ppm, about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, or about 700 ppm). In some embodiments, in a food composition, the steviol glycoside formulation is present in the range of about 0.0005% to about 1% (e.g., about 0.0005%, about 0.001%, about 0.005%, about 0.1%, about 0.2%, about 0.3%, about 0.4%, about 0.5%, about 0.6%, about 0.7%, about 0.8%, about 0.9%, or about 1.0%) by weight of the total weight of the food composition.

In some embodiments, the orally consumable product comprising any one of the steviol glycoside formulations described herein is an animal feed product for livestock, companion animals and/or aquaculture. In some embodiments, the livestock is cattle, swine and/or poultry. In some embodiments, in an animal feed product, the steviol glycoside formulation is present in a concentration of about 50-800 ppm (e.g., about 50 ppm, about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, or about 800 ppm). In some embodiments, the animal feed product further comprises a hydrocolloid or erythritol.

In some embodiments, any one of the orally consumable products described herein further comprises a component selected from the group consisting of sucrose, aroma compounds, flavoring compounds and mixtures thereof. In some embodiments, any one of the orally consumable products described herein further comprises tocopherols in an amount of at least about 5 ppm. In some embodiments, any one of the orally consumable products described herein further comprises at least one stabilizing agent selected from the group consisting of citric acid, sodium benzoate, t-butyl hydroquinone, ascorbyl palmitate, propyl gallate, and combinations thereof. In some embodiments, any one of the orally consumable products described herein further comprises a moisture containing ingredient. In some embodiments, the moisture ingredient is an emulsion. In some embodiments, any one of the orally consumable products described herein further comprises a chelating agent.

In some embodiments, any one of the orally consumable products described herein can also have at least one additional sweetener. The at least one additional sweetener can be a natural high intensity sweetener, for example. The additional sweetener can be selected from a Stevia extract, a steviol glycoside, stevioside, rebaudioside B, rebaudioside C, rebaudioside D, rebaudioside F, dulcoside A, rubusoside, steviolbioside, sucrose, high fructose corn syrup, fructose, glucose, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, mogroside IV, siamenoside I, mogroside V, monatin, thaumatin, monellin, brazzein, L-alanine, glycine, Lo Han Guo, hernandulcin, phyllodulcin, trilobtain, and combinations thereof. In some embodiments, any one of the orally consumable products described herein does not have a sweetener in addition to a steviol glycoside formulation provided herein.

In some embodiments, any one of the orally consumable products described herein can also have at least one additive. The additive can be, for example, a carbohydrate, a polyol, an amino acid or salt thereof, a polyamino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements can be, for example, nutrients, vitamins, minerals, fiber, fatty acids, herbs, botanicals, amino acids, and metabolites.

As used herein, “nutraceutical(s)” refers to compounds, which includes any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.). Any suitable nutraceutical known in the art may be used. In some embodiments, nutraceuticals can be used as supplements to food and beverages and as pharmaceutical formulations for enteral or parenteral applications which may be solid formulations, such as capsules or tablets, or liquid formulations, such as solutions or suspensions.

In some embodiments, dietary supplements and nutraceuticals can further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels can be used in a number of applications. For example, gels can be used in foods, paints, and adhesives. Gels that can be eaten are referred to as “edible gel compositions.” Edible gel compositions typically are eaten as snacks, as desserts, as a part of staple foods, or along with staple foods. Examples of suitable edible gel compositions can be, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids can be, for example, water, dairy fluids, dairy analogue fluids, juices, alcohol, alcoholic beverages, and combinations thereof. Examples of suitable dairy fluids can be, for example, milk, cultured milk, cream, fluid whey, and mixtures thereof. Examples of suitable dairy analogue fluids can be, for example, soy milk and non-dairy coffee whitener.

As used herein, the term “gelling ingredient” refers to any material that can form a colloidal system within a liquid medium. Examples of suitable gelling ingredients can be, for example, gelatin, alginate, carageenan, gum, pectin, konjac, agar, food acid, rennet, starch, starch derivatives, and combinations thereof. It is well known to those in the art that the amount of gelling ingredient used in an edible gel mix or an edible gel composition can vary considerably depending on a number of factors such as, for example, the particular gelling ingredient used, the particular fluid base used, and the desired properties of the gel.

Gel mixes and gel compositions of the present disclosure can be prepared by any suitable method known in the art. In some embodiments, edible gel mixes and edible gel compositions of the present disclosure can be prepared using other ingredients in addition to the gelling agent. Examples of other suitable ingredients can be, for example, a food acid, a salt of a food acid, a buffering system, a bulking agent, a sequestrant, a cross-linking agent, one or more flavors, one or more colors, and combinations thereof.

In certain embodiments that can be combined with any of the preceding embodiments, the orally consumable products can further include one or more additives selected from a carbohydrate, a polyol, an amino acid or salt thereof, a poly-amino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

The compositions can be used “as-is” or in combination with other sweeteners, flavors and food ingredients. For use in domestic applications, particularly as a replacement for sugar in beverage sweetening, it is desirable in some embodiments that the compositions according to the present disclosure include a bulking agent so that an equivalent sweetness to that provided by, for example, a teaspoonful of sugar is provided by an amount which can conveniently be handled. Any suitable soluble and edible material can be used, for example, a carbohydrate such as sucrose itself, especially transformed sugar of low density, dextrose, or sorbitol or a dextrin such as spray-dried maltodextrin. While the substances will add to the caloric value of the composition, the total will still be considerably smaller than that of the amount of sugar providing an equivalent sweetness. Alternatively, the sweetening composition may be prepared in a tablet form.

Compositions provided herein are usually stable at pH values in the range of from 2 to 10, especially 3 to 8. Dry compositions, such as powders, granules or tablets can be stable indefinitely when stored under dry conditions at room temperature. Compositions in the form of aqueous solutions can be stable indefinitely when frozen. If a preservative such as benzoic acid or its salts, sulphur dioxide or sodium meta-bisulphite is added to such a composition, it may be stored almost indefinitely at room temperature. The compositions therefore can have a long shelf-life when incorporated into soft drinks or fruit juices, or other similar food compositions containing preservatives. The limitation on the use of sugar may also positively contribute to the long shelf-life of the products provided herein.

### Food Additives Flavor Enhancement or Aroma Enhancement

Food compositions comprising the inventive formulations provided herein may further comprise components selected from the group consisting of additional sweeteners or sweet-tasting compounds, aroma compounds, flavoring compounds, and their mixtures. Such additives may also specifically include hydrocolloids such as pectins, gelatin, carrageenan, or gums (Arabic, guar, locust bean) for dressings, jams, jellies, confections and the like. Other additives to food, feed or beverage compositions include chelating agents whose addition is designed to protect against enzymatic reactions and may specifically include ethylenediaminetetraacetic acid (EDTA).

Aroma compounds and flavor enhancing agents are well known in the art can be added to the compositions provided herein. These flavoring agents can be chosen from synthetic flavoring liquids and/or oils derived from plants leaves, flowers, or fruits. Representative flavoring liquids include: artificial, natural or synthetic fruit flavors such as eucalyptus, lemon, orange, banana, grape, lime, apricot, and grapefruit oils, fruit essences including apple, strawberry, cherry, orange, pineapple, and so forth, bean- and nut-derived flavors such as coffee, cocoa, cola, hazelnut, peanut or almond, and root-derived flavors such as licorice or ginger.

The following examples illustrate various embodiments of the disclosure. It will be understood that the disclosure is not limited to the materials, proportions, conditions and procedures set forth in the examples, which are only illustrative.

### EXAMPLES

### Example 1

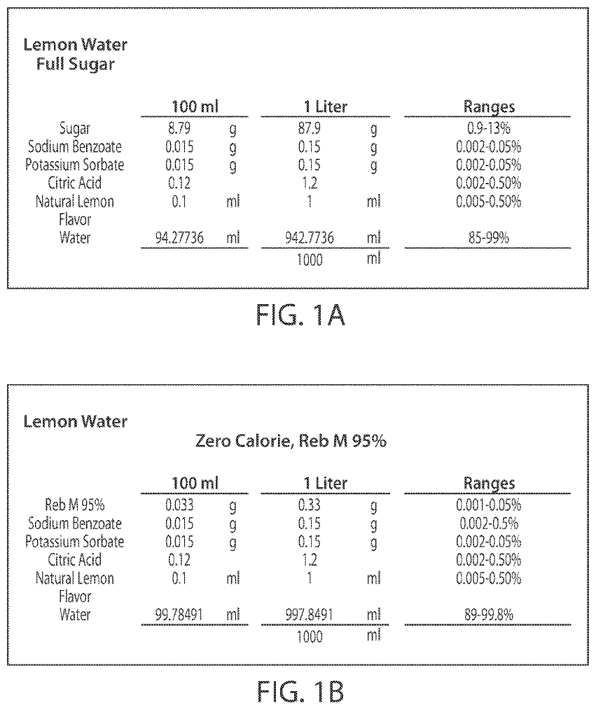

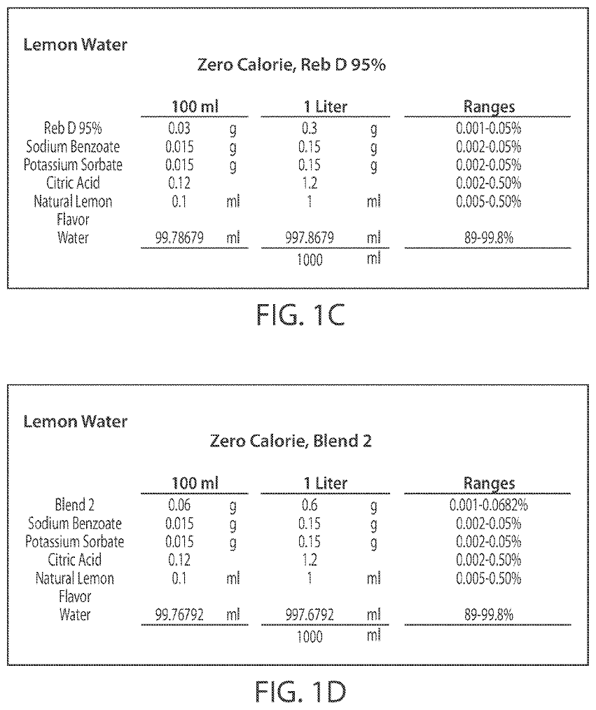

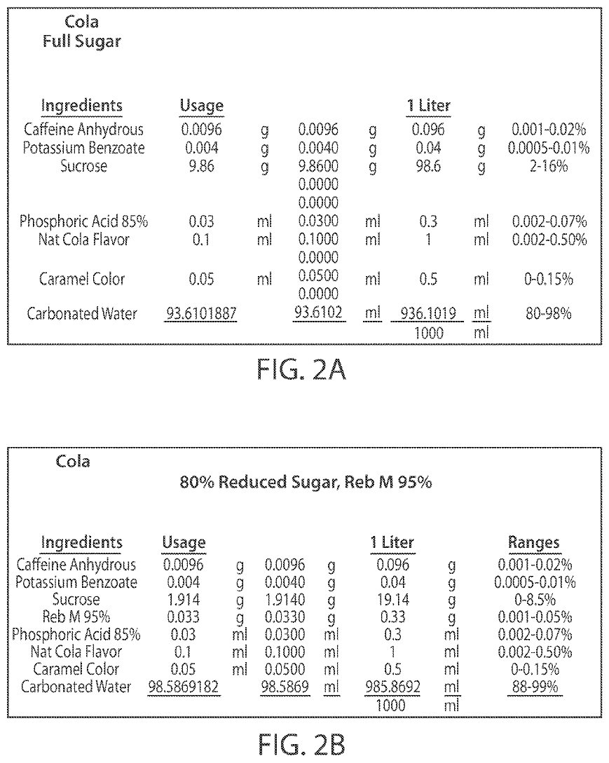

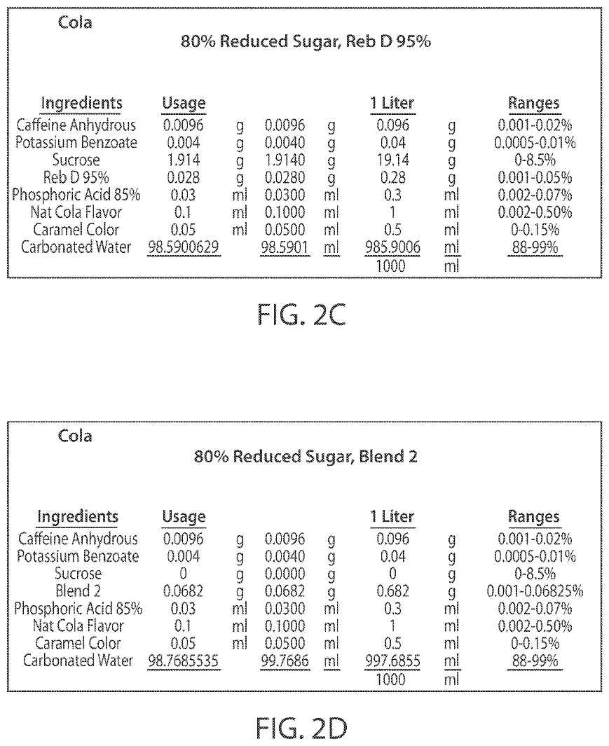

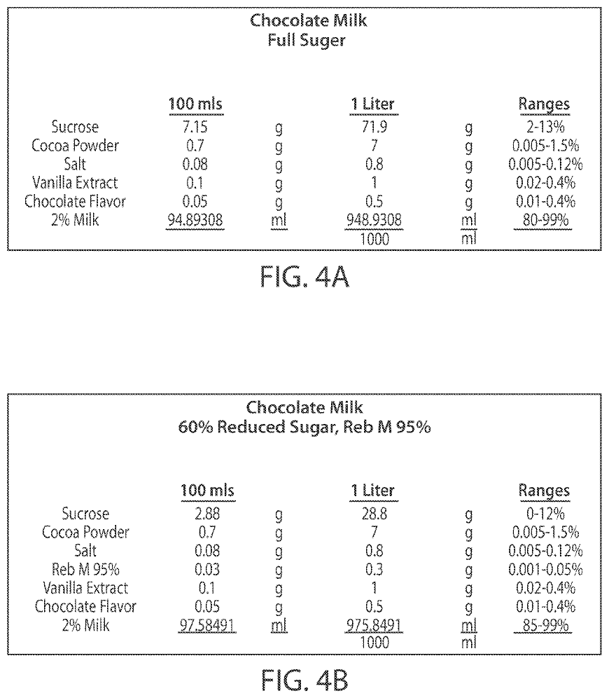

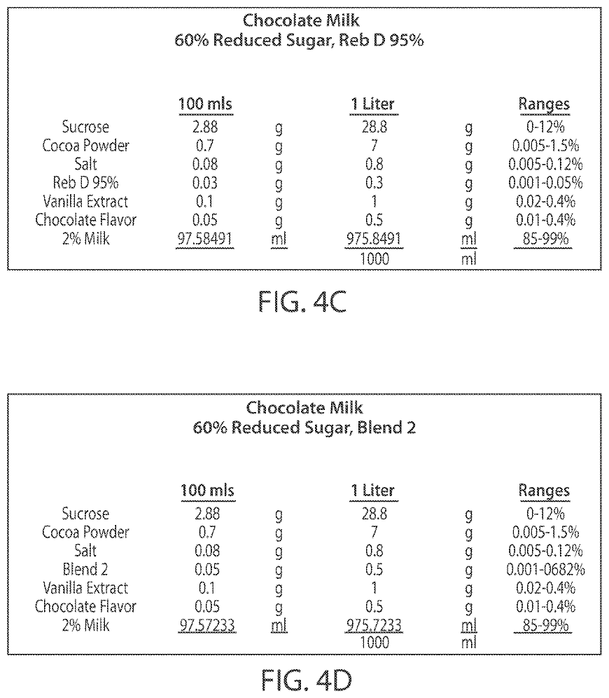

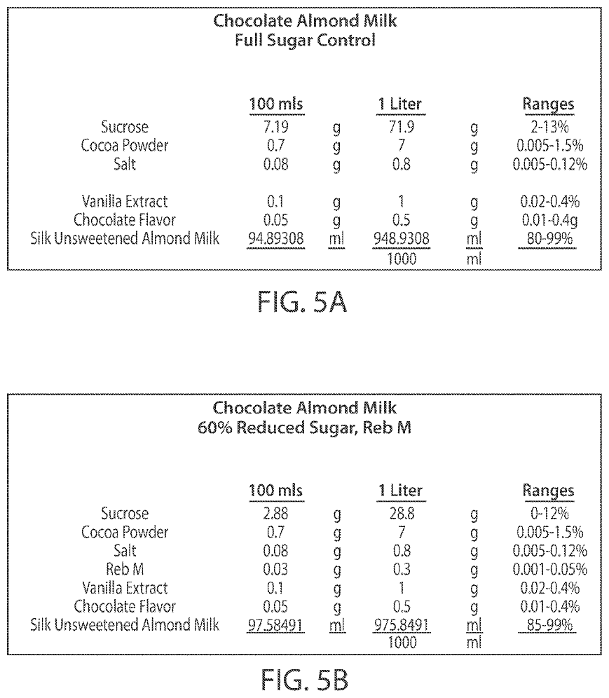

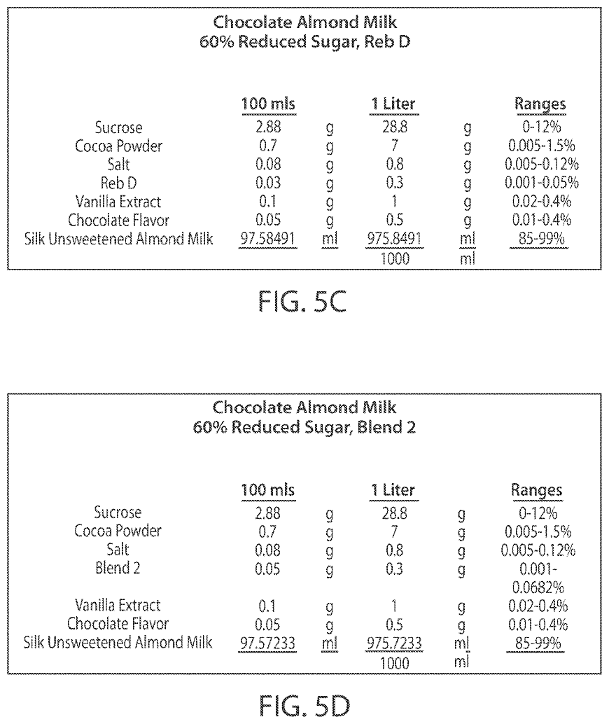

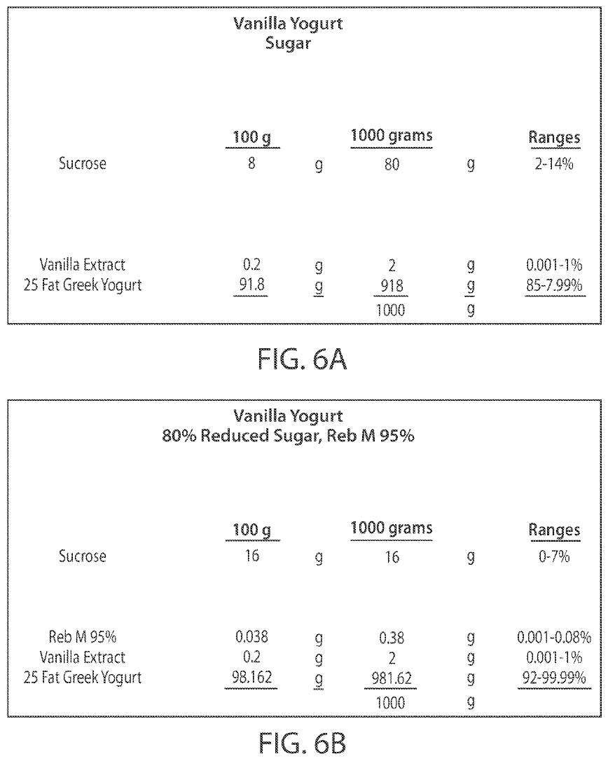

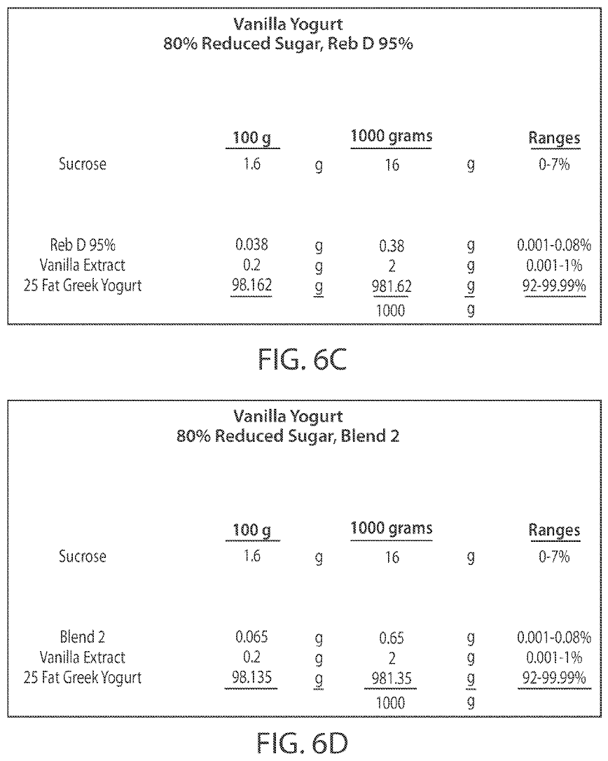

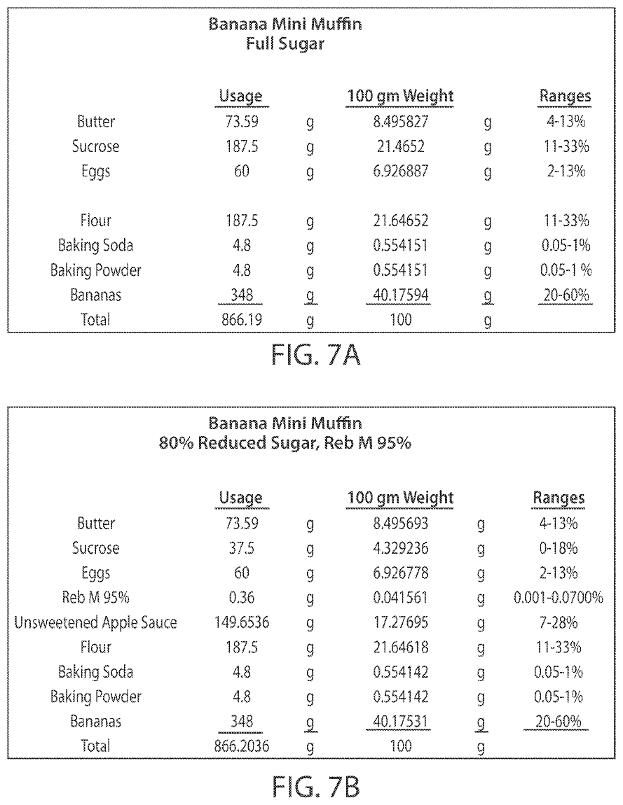

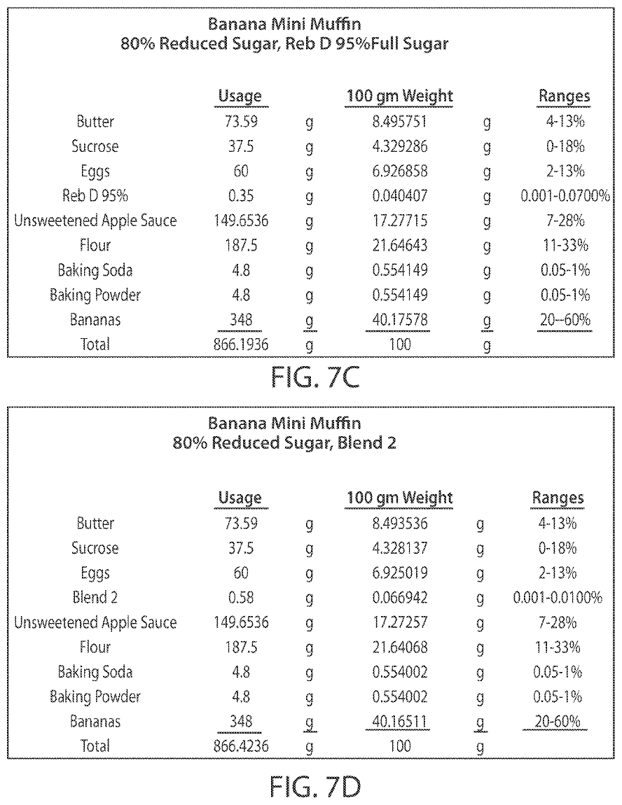

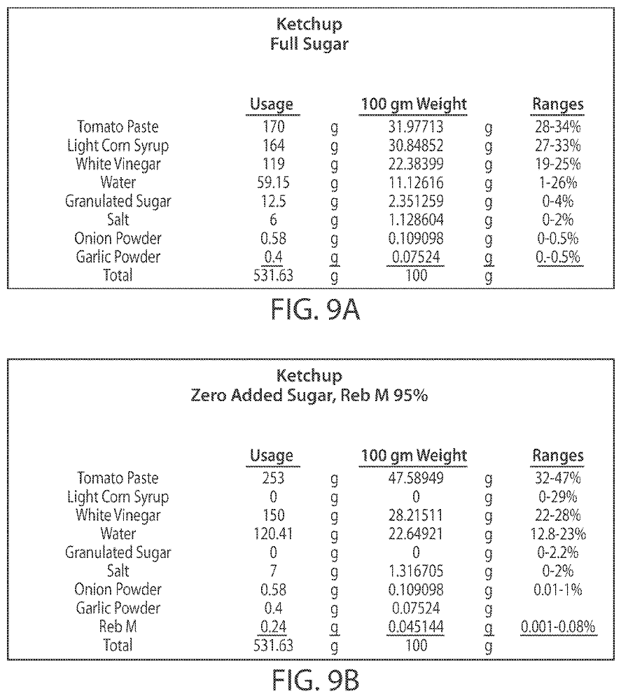

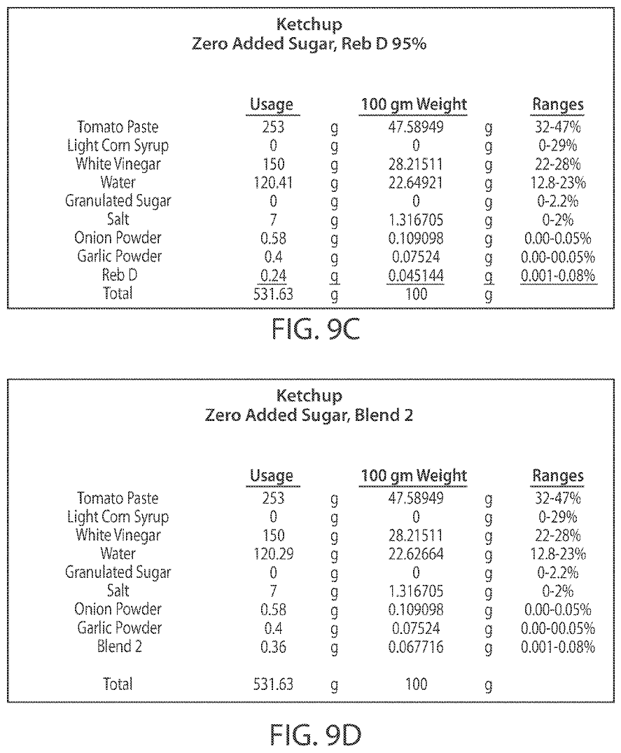

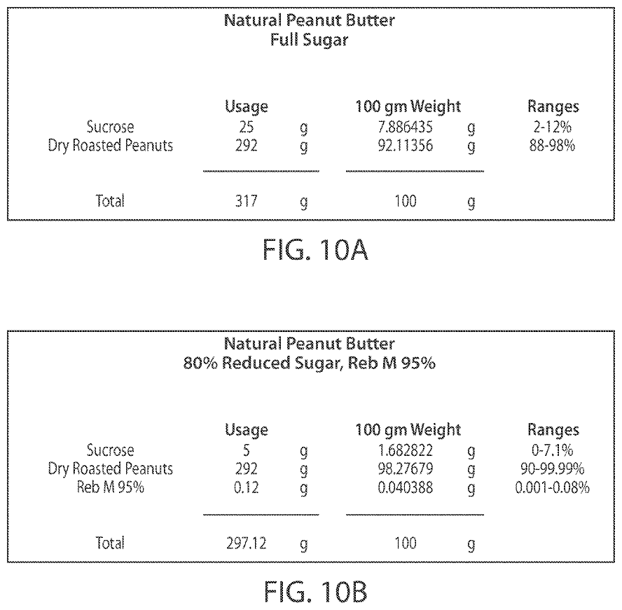

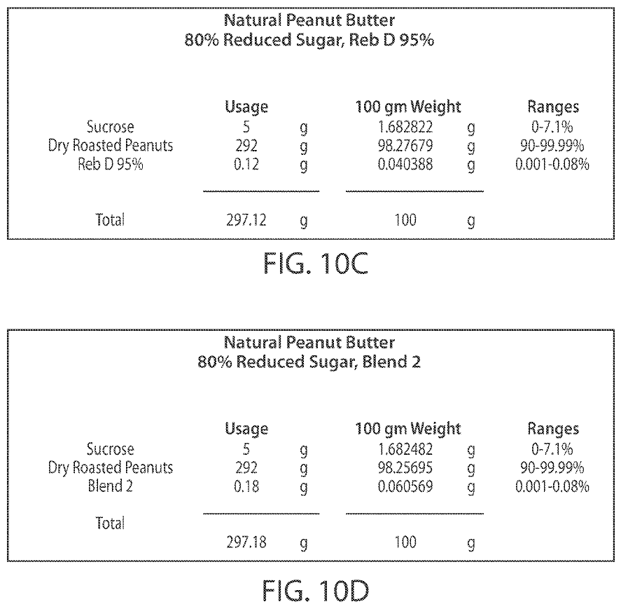

According to the present disclosure, the creation of food products incorporating the sweetening blends provided herein can be provided in several food category (FIGS. 1A-10D). Food or beverages that can contain the inventive rebaudioside formulations include baked goods and baked good mixes (e.g., cakes, brownies, muffins, cookies, pastries, pies, and pie crusts), shortening and oil products (e.g., margarines, salad dressings and mayonnaise), companion animal feed, dairy products and artificial dairy products (e.g., butter, ice cream and other fat-containing frozen desserts, yogurt, and cheeses, including natural cheeses, processed cheeses, cream cheese, cottage cheese, cheese foods and cheese spread, milk, cream, sour cream, buttermilk, and coffee creamer), meat products (e.g., hamburgers, hot dogs, wieners, sausages, bologna and other luncheon meats, canned meats, including pasta/meat products, stews, sandwich spreads, and canned fish), meat analogs, tofu, and various kinds of protein spreads, sweet goods and confections (e.g., candies, chocolates, chocolate confections, frostings, and icings, syrups, cream fillings, and fruit fillings), nut butters and various kinds of soups, dips, sauces and gravies. Each of the above examples comprise different embodiments of the current disclosure. The formulations of the present disclosure are generally completely soluble in water and may be used in hot or cold food and beverages to give a sweetness equivalent to that of sugar.

The preferred formulations the present disclosure were developed with the appropriate level of a particular rebaudioside blend in order to deliver the targeted sweetness levels on a per serving basis. The amount added varied between different applications due to the differences in serving size. For ease of addition and in order to achieve homogeneous distribution at the desired dilution in edible materials, the compositions were formulated in the conventional manner with solid or liquid non-toxic carrier or diluents. For example, solid compositions may take the form of tablets or powders using edible solid carriers such as maltodextrins, starch or nutritive proteins (e.g. soy protein); or the formulations provided herein may be fixed with sucrose to provide a “fortified” sugar. Liquid compositions may take the form of aqueous solutions or of suspensions in other non-toxic liquids such as aqueous ethanol, glycerol and edible oils, and may be used, for example, for spraying.

### Soy Milk

Soymilk can be prepared in different ways. In one example, a sweetening rebaudioside formulation is folded into full-fatted soy flour or added separately as needed. The soymilk is formulated by first dissolving the soy flour into water, mixing, and processing to inactivate the enzymes. The soy base is filtered to remove additional solids and degassed. The remaining ingredients are added and mixed, and the product is homogenized in a two-stage homogenizer, then processed through an Ultra High Temperature (UHT) thermal processing unit. The resulting product is packed and refrigerated with a typical shelf life of 12 weeks.

Following is a formulation as provided in Table 4. See also FIG. 12 for a process flow diagram. Note that the rebaudioside blends can be used instead of the sucrose listed. Given their potency as a sweetener, they could require a fraction of the total amount of sucrose otherwise needed and could act as a complete replacement.

TABLE 4

| Vanilla Soymilk | % |
|---|---|
| Water | 88.122 |
| Enriched Soy Flour | 6.786 |
| Full Fat Soymilk. | 0.600 |
| *Sucrose - or Rebaudioside | 3.400 |
| Blend at a fraction of total sugar | |
| Carrageenan | 0.022 |
| Cellulose Gum | 0.350 |
| Salt | 0.040 |
| Calcium Carbonate | 0.350 |
| Natural and Artificial Flavors | 0.330 |
| TOTAL | 100.000 |

The example used can also be applied to different types of homogenization and thermal processing units (direct steam, indirect steam, etc.). Different soymilk flavors, including plain, chocolate, apple, orange, berry, etc. can be prepared in the same manner.

The resulting product was found to have acceptable flavor and mouth “feel” properties in comparison to soymilk made from flour processed the same way but without a rebaudioside blend as provided herein.

Another example is to use isolated soy protein and to add a rebaudioside mixture to the isolate in lieu of sucrose. Following is a formulation as provided in Table 5.

TABLE 5

| Vanilla Soymilk | % |
|---|---|
| Water | 88.058 |
| *Sucrose or Rebaudioside | 3.500 |
| Blend at a fraction of total sugar | |
| Isolated Soy Protein | 2.700 |
| Maltodextrin | 3.500 |
| 11% Soybean Oil | 1.500 |
| Carrageenan | 0.022 |
| Cellulose gum | 0.350 |
| Salt | 0.040 |
| Natural & Artificial Flavors | 0.330 |
| TOTAL | 100.000 |

The example provided above can also be applied to different types of homogenization and thermal processing units (direct steam, indirect steam, etc.). Different soymilk flavors, including plain, chocolate, apple, orange, berry, etc. can be prepared in the same manner.

### Animal Feed

To make the animal feed (e.g., see FIG. 14), combine all ingredients in a very large pan and add the cooled cooked white rice. Blend 9 eggs in a blender with shells, thoroughly homogenize egg and shells and add to mix. Mix well and completely with your hands until all the ingredients are thoroughly combined. The ingredients are enough for about three meat loafs. Cook meat loafs in over preheated to 350 F for about 1 hour or until done. The meatloaf can be broken into desirable amounts dependent upon dog to be fed and refrigerate/freeze the rest.

### Example 2

### Margarine Type Spreads

A typical margarine process is that the water, salt, sodium benzoate, and butter flavor are mixed as an aqueous phase. A milk ingredient, such as whey powder, sodium caseinate, or milk powder, is added to the aqueous phase. The oils, lecithin, mono and diglycerides, vitamins, and sweeteners, including a Stevia blend as provided herein, are mixed, combined with the aqueous phase, and mixed. The mixed emulsion is passed through a series of scraped surface heat exchangers, pin mixers, and resting tubes to achieve a desired fill temperature and consistency.

### Example 3

### Cookie Dough

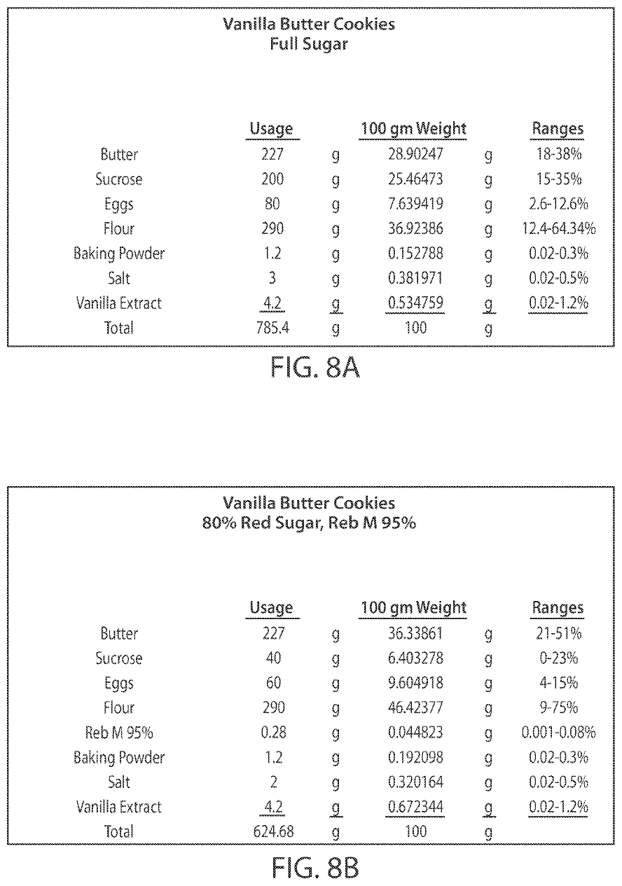

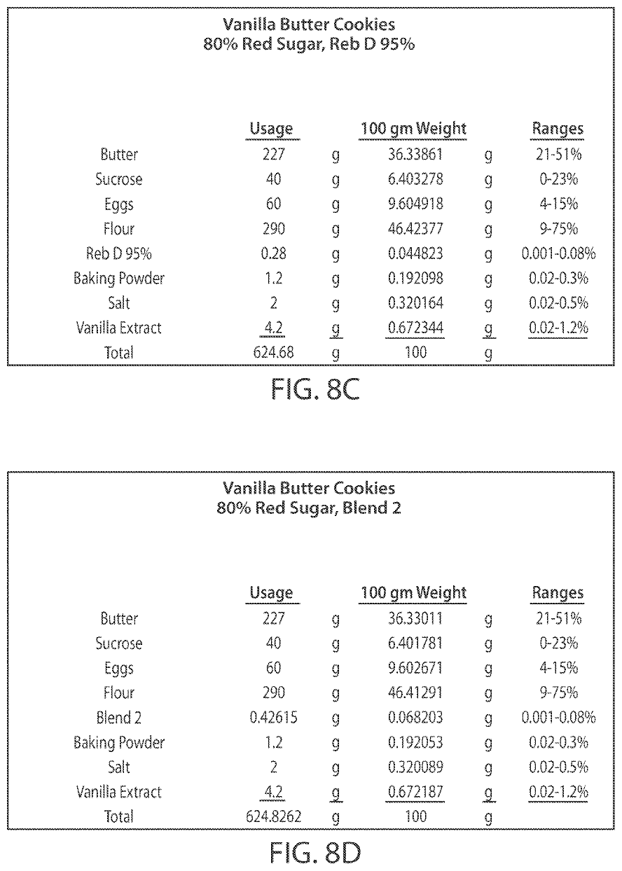

disclosure sweetener blend as provided herein can also be incorporated into food products, including cookies. The recipe for vanilla butter cookies is provided in FIGS. 8A-8D for such utilization.

### Example 4

### Reduction or Replacement of Sugar in Beverages

The lemon water examples shown in FIGS. 1B-1D are created based upon the lemon water shown in FIG. 1A (control), which is a typical lemon water constituting lemon drinks and comprises sugar (in an amount of 8.79 g), natural lemon, water, and preservatives sodium benzoate and citric acid in a total volume of 100 ml. As shown in FIG. 1B, Rebaudioside M is added in an amount of 0.033 g. The other components are present in the same amounts in the total volume of 100 ml, with the exception of the added sugar in the example shown in FIG. 1A.

As shown in FIG. 1C, another exemplary formulation is created similarly to that of FIG. 1B, with Rebaudioside M being replaced by Rebaudioside D, and again added in an amount of 0.033 g, with the other components present in the same amounts in the total volume of 100 ml.

### Example 5

### Use of Preferred Embodiments of the Present Formulations in Lemon Water, Replacing Sugar

FIG. 1D shows one of the formulations provided herein replacing the components sugar, Rebaudioside M, and Rebaudioside D in the previous examples (FIGS. 1A-1C).

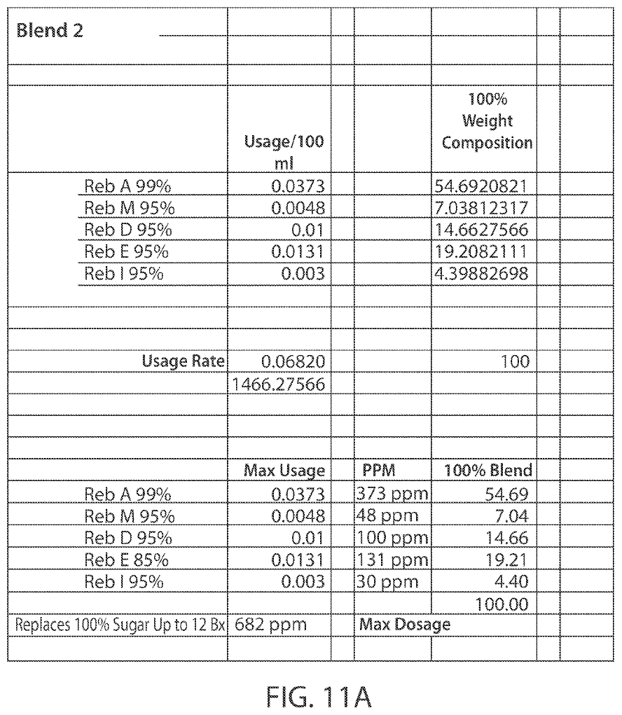

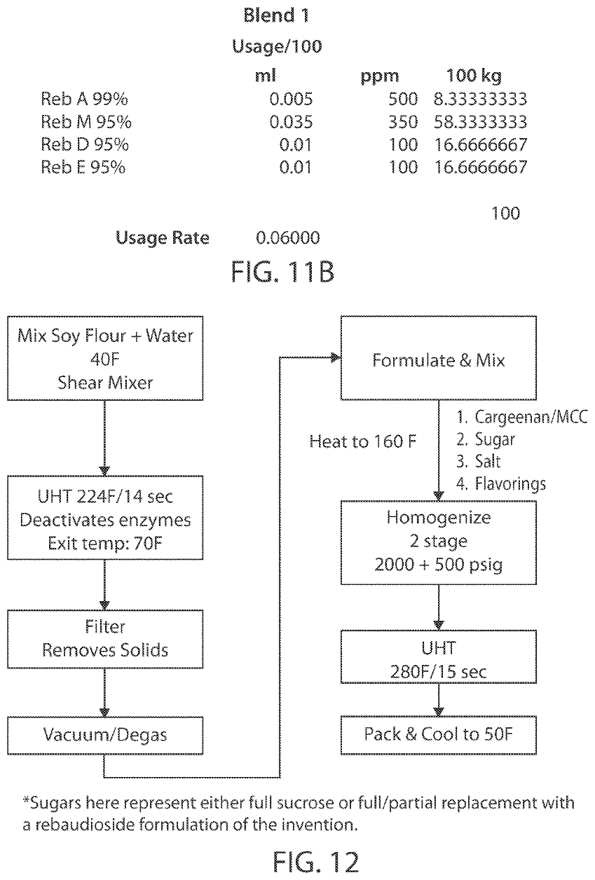

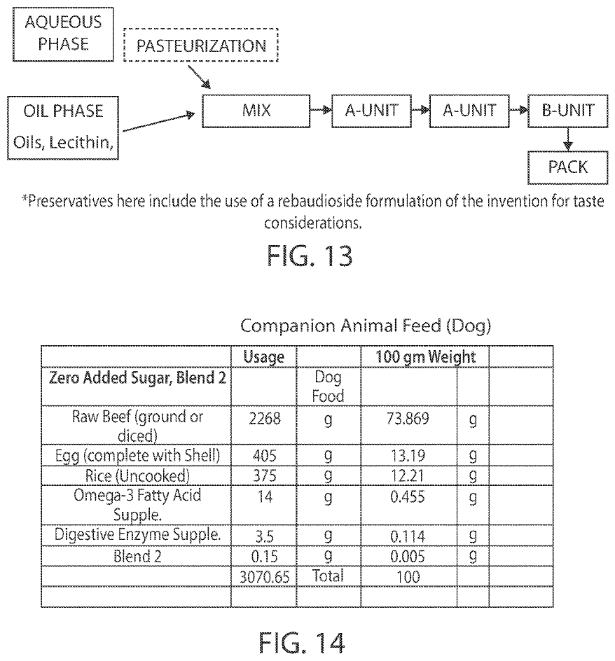

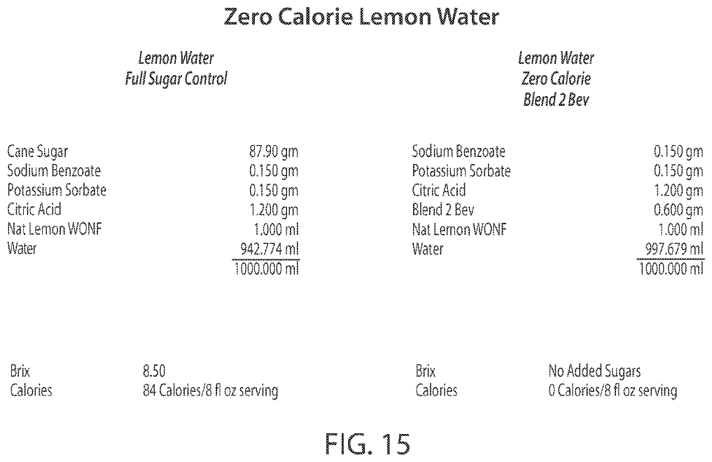

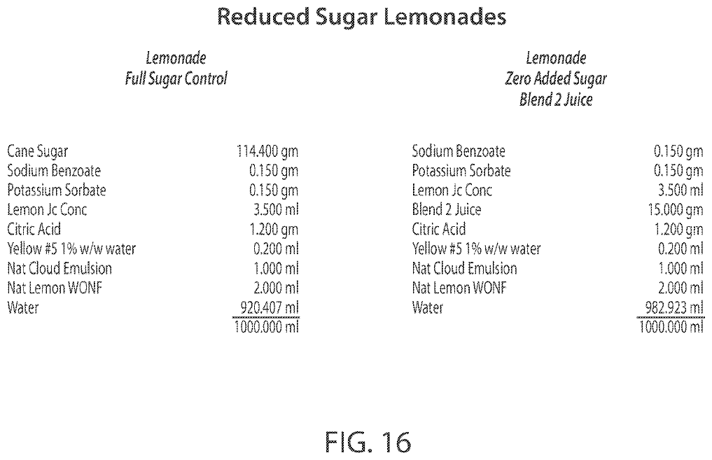

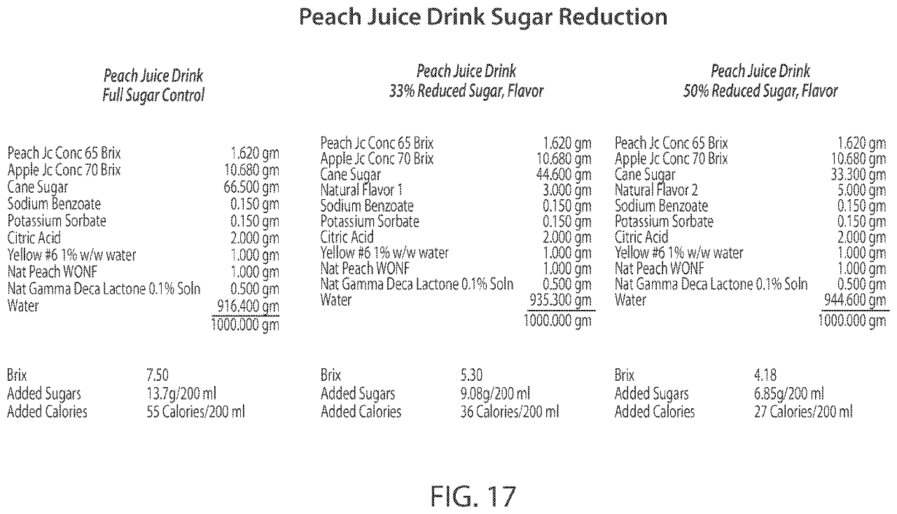

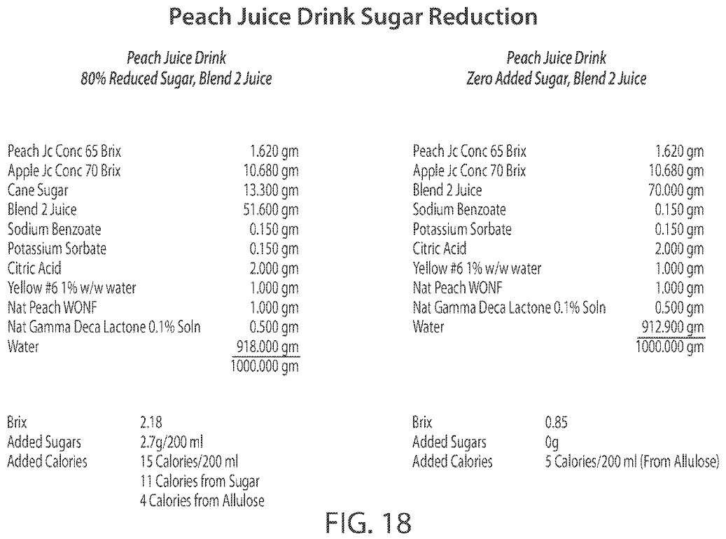

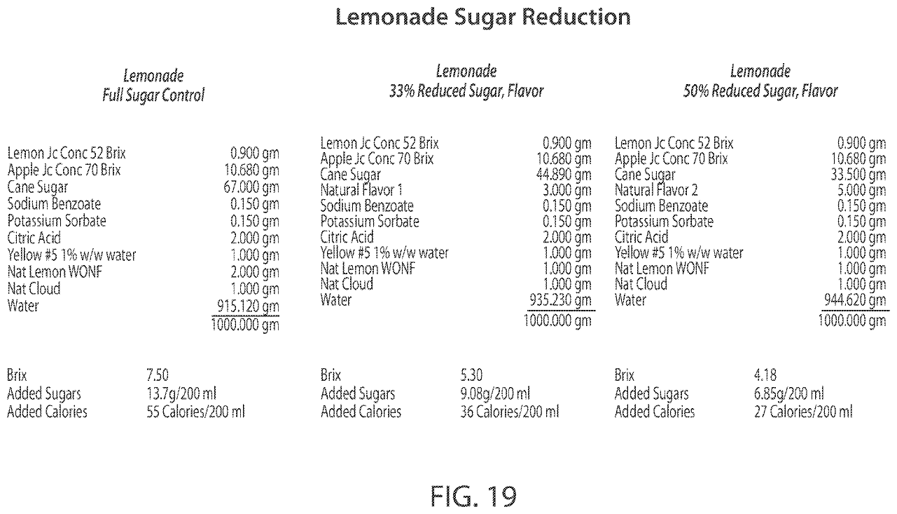

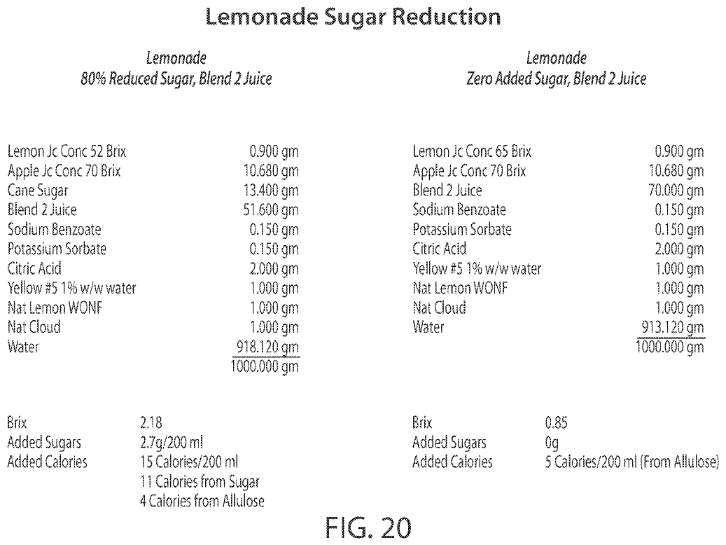

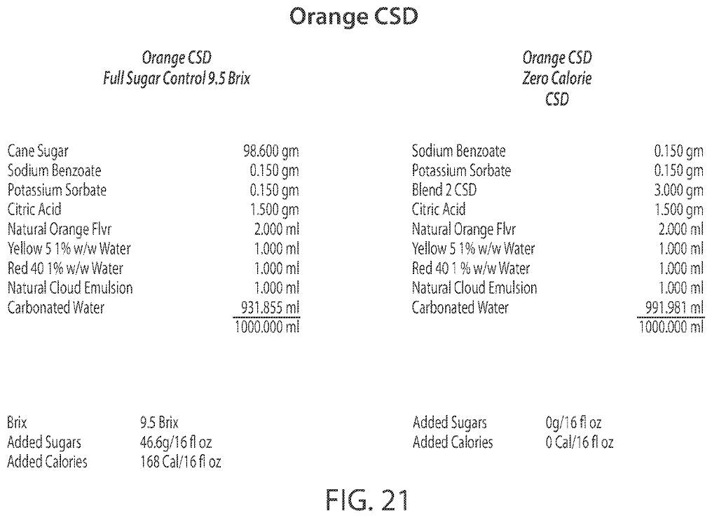

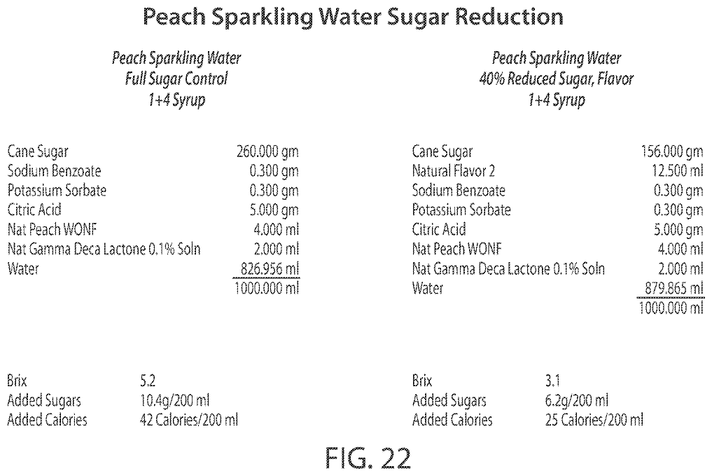

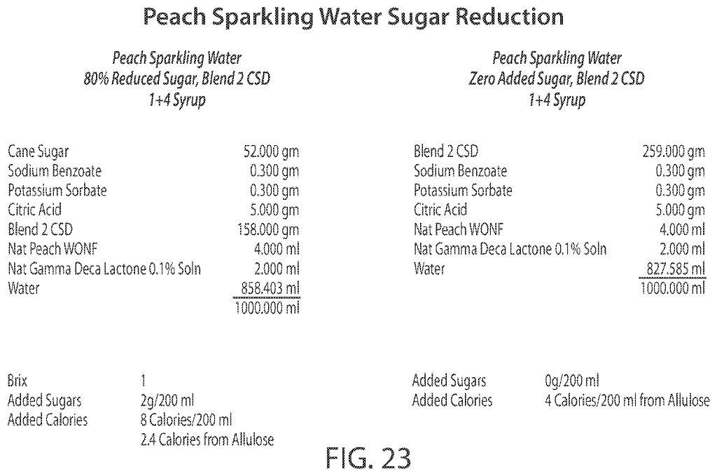

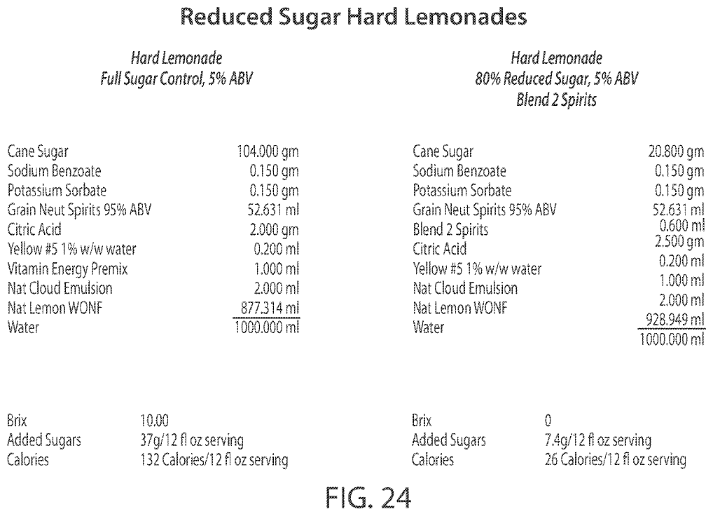

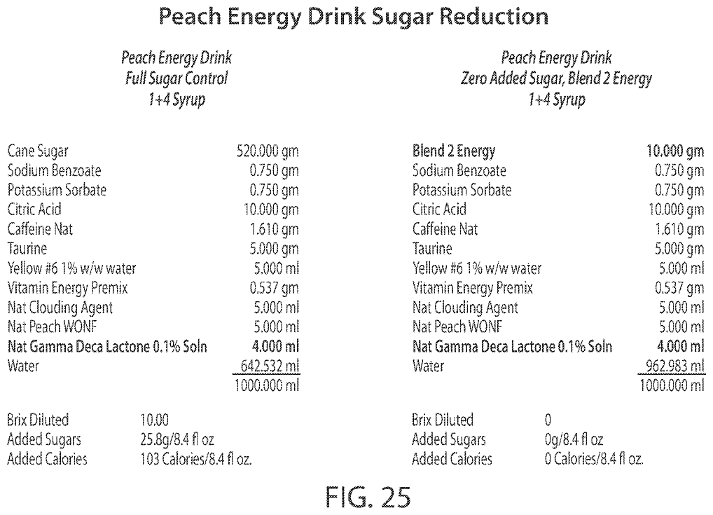

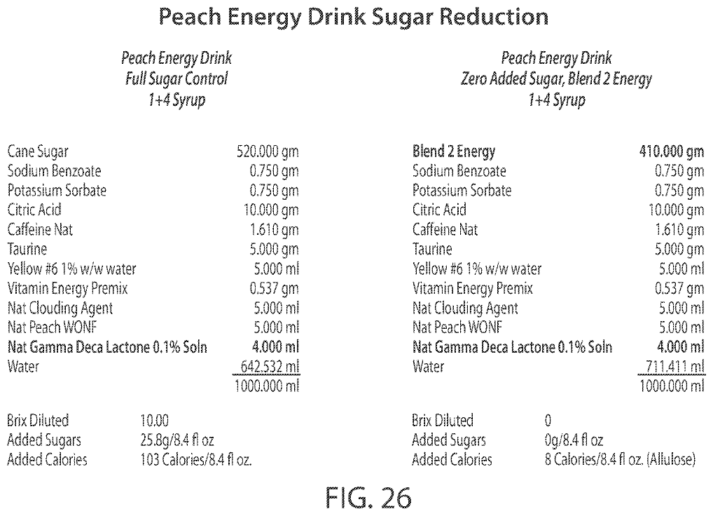

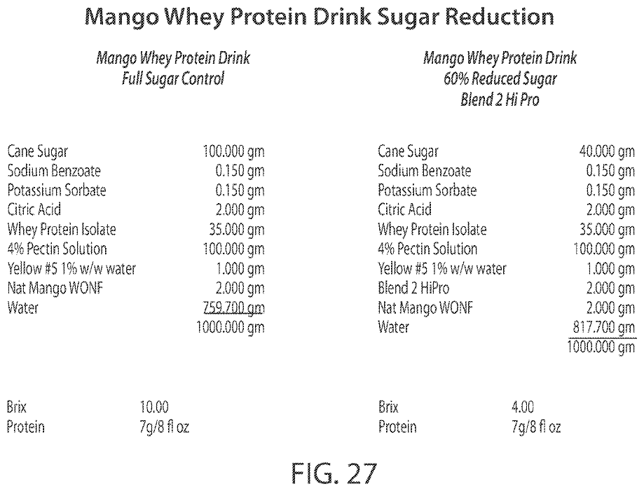

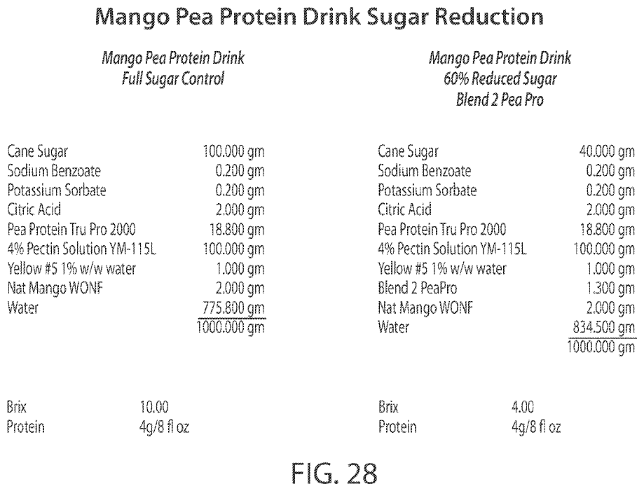

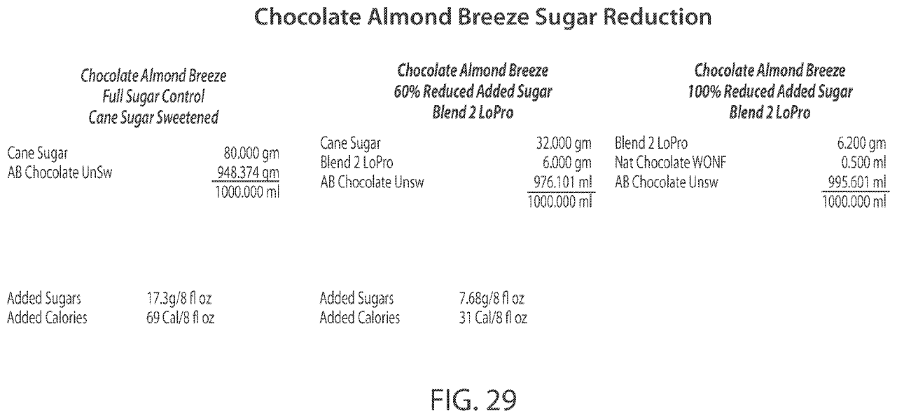

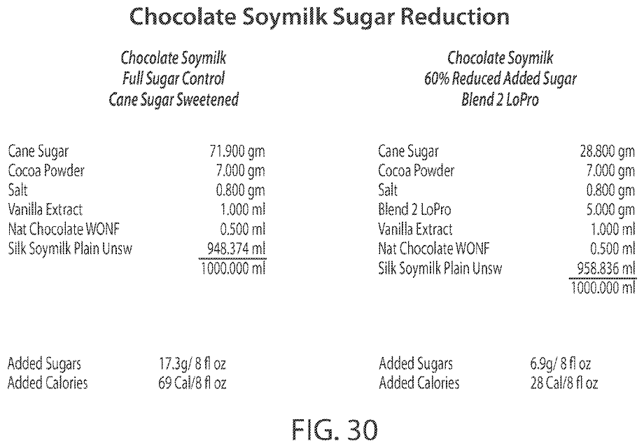

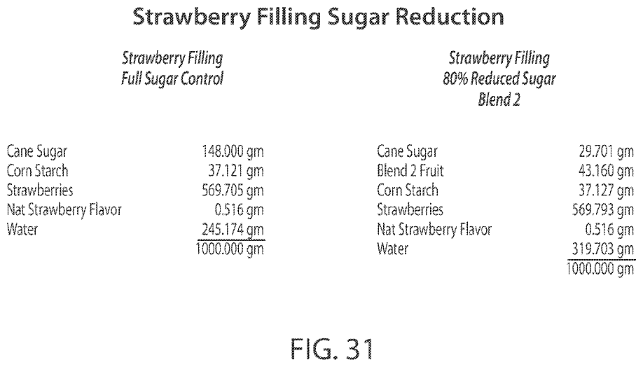

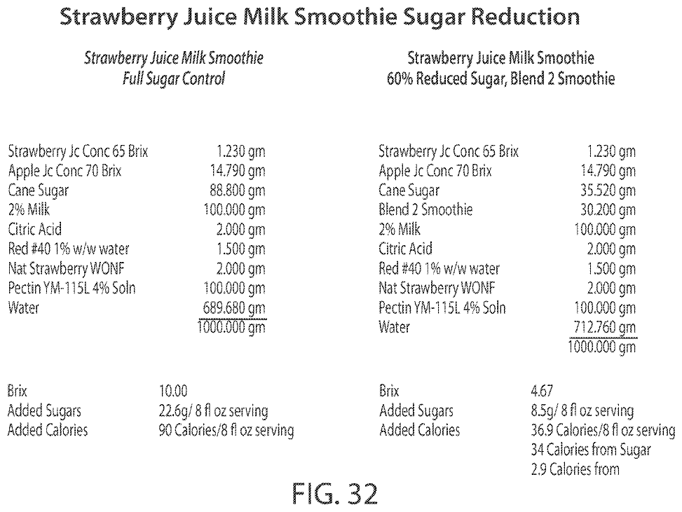

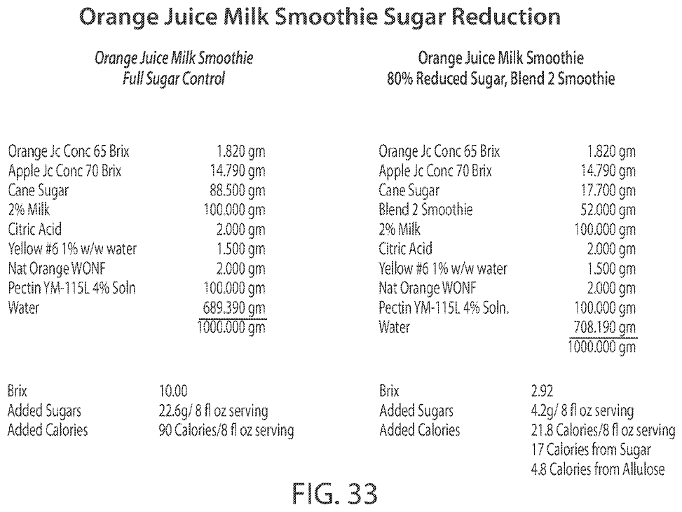

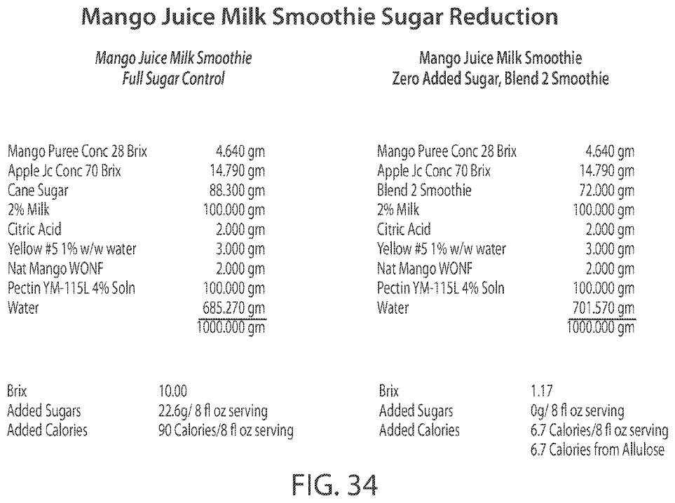

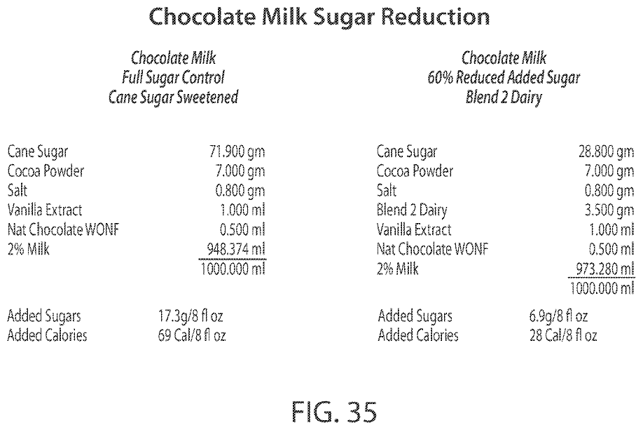

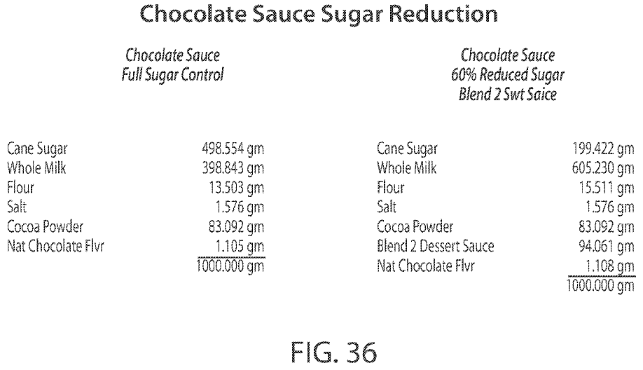

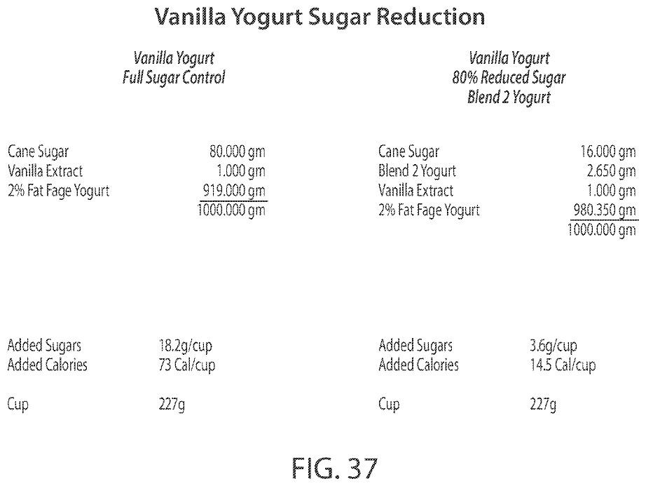

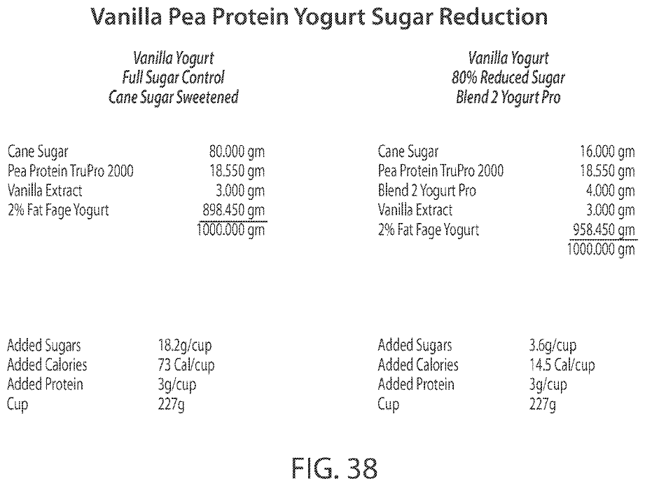

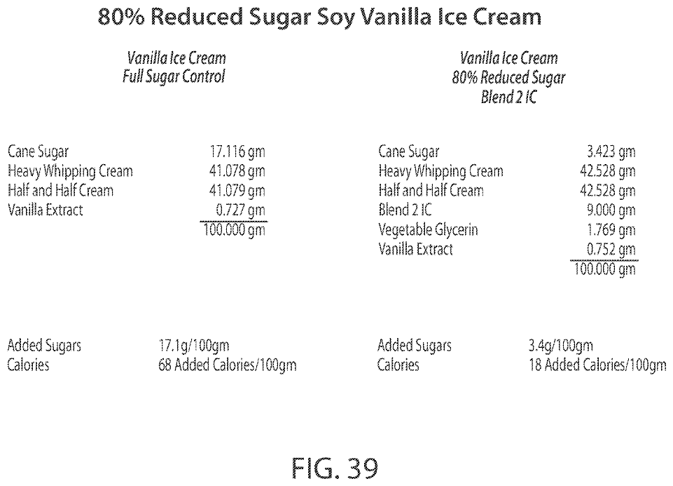

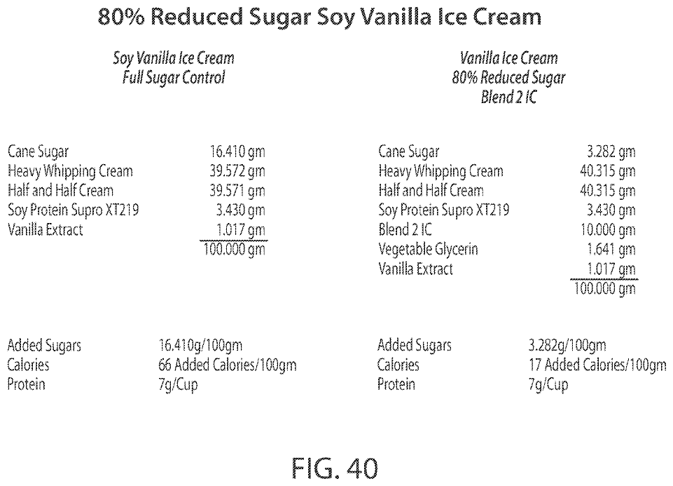

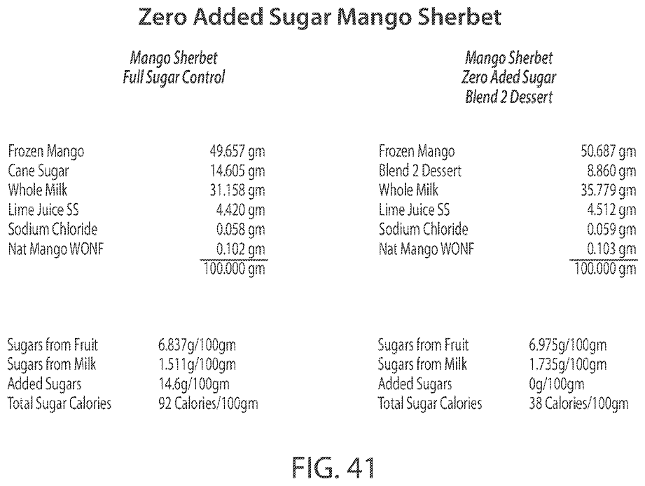

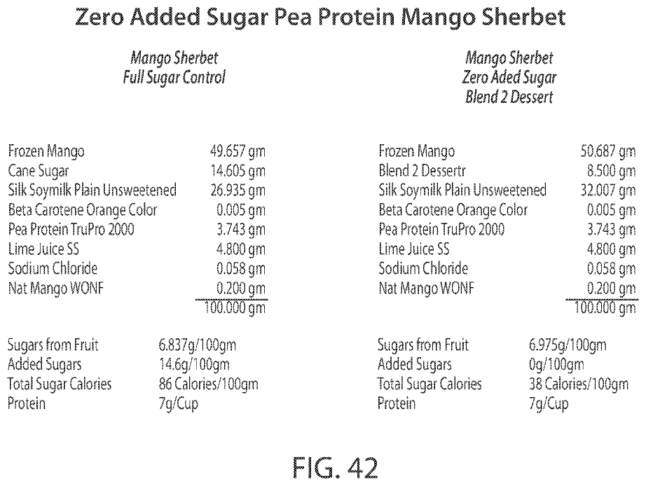

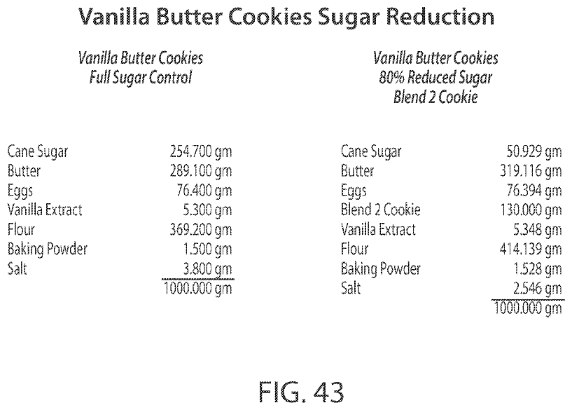

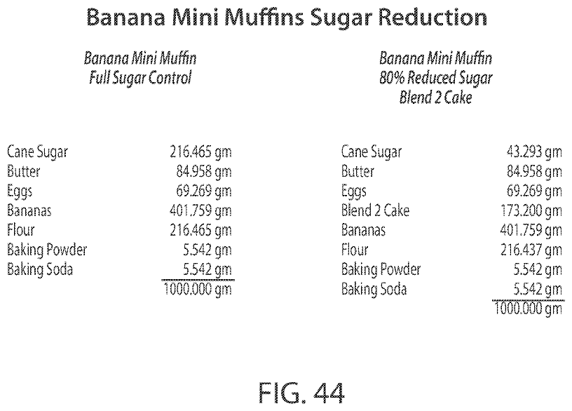

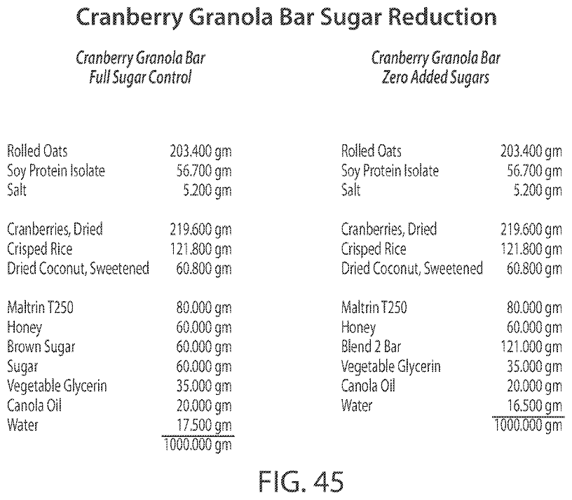

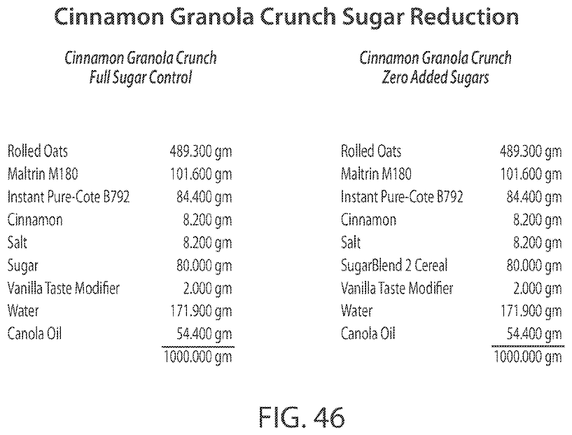

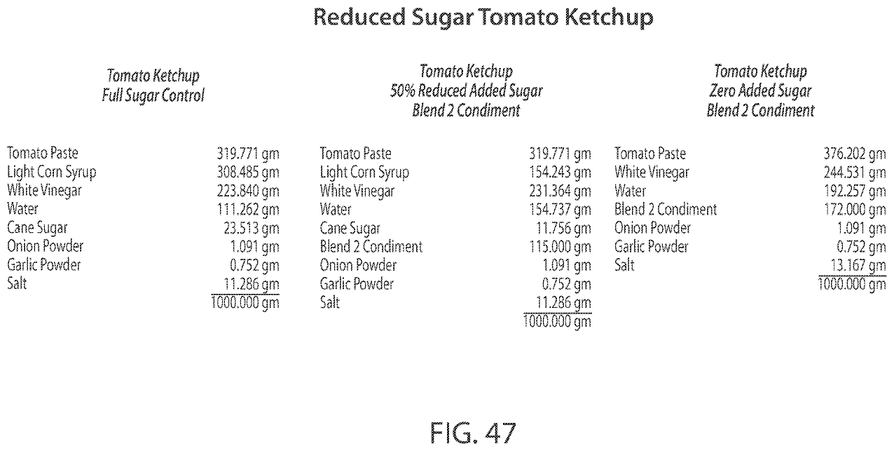

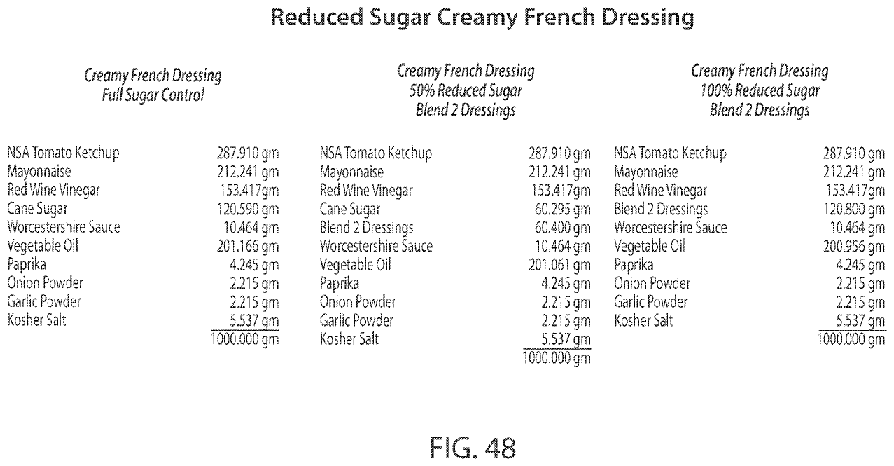

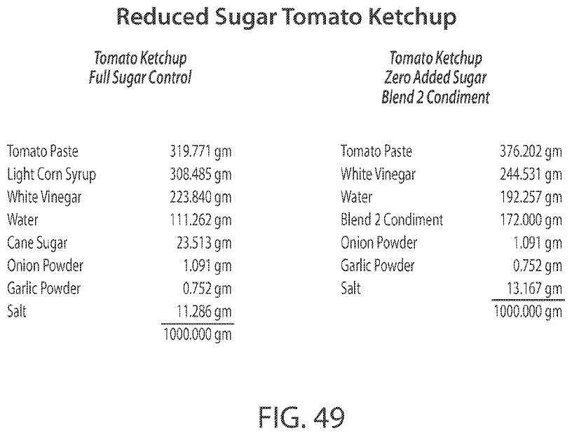

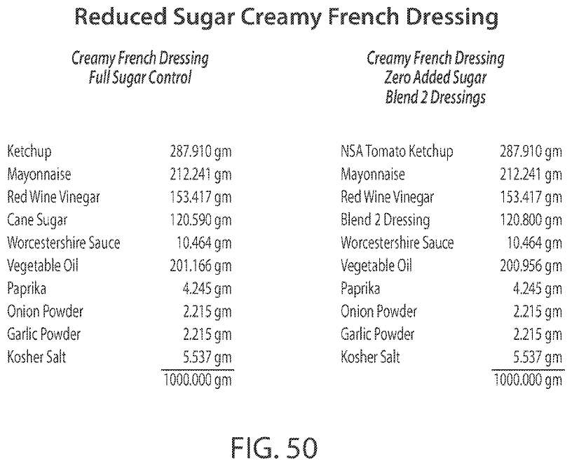

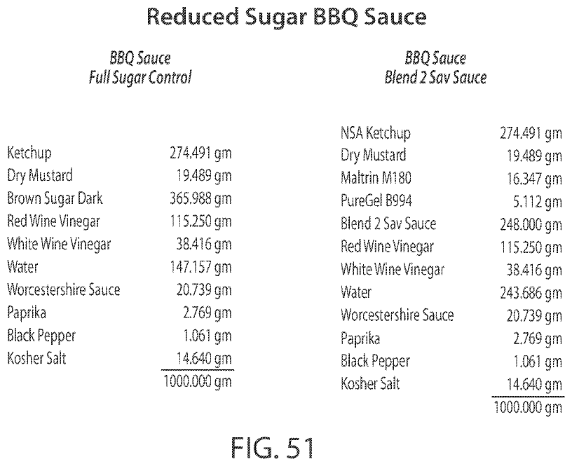

In this example, use of one of a formulation as provided herein to make lemon water is shown in FIG. 1D. The rebaudioside formulation, labelled Blend 2, was added to the same components of the previous example, in which the Blend 2 formulation replaced the sugar, the Rebaudioside M and the Rebaudioside D of FIGS. 1A-1C. The formulation Blend 2 comprises the following components, as shown in FIG. 11A, in the following amounts:
Rebaudioside A (99% purity), 0.0373 g
Rebaudioside M (95% purity), 0.0048 g
Rebaudioside D (95% purity), 0.01 g
Rebaudioside E (95% purity), 0.0131 g
Rebaudioside 1 (95% purity), 0.003 g for a total volume of 100 ml lemon water.

Each food or beverage provided in the examples or shown in the figures exhibits a rounded and complete flavor profile and excellent mouthfeel in comparison to full sucrose versions of the same food product or beverage.

The use of the inventive sweetener formulations can also be used for a variety of other beverages including in the preparation of juice drinks from other fruits, such as apples, lemons, apricots, cherries, pineapples, mangoes, for example. It should be noted that the data shown throughout represents the results obtained by using sweetener formulations as provided herein.

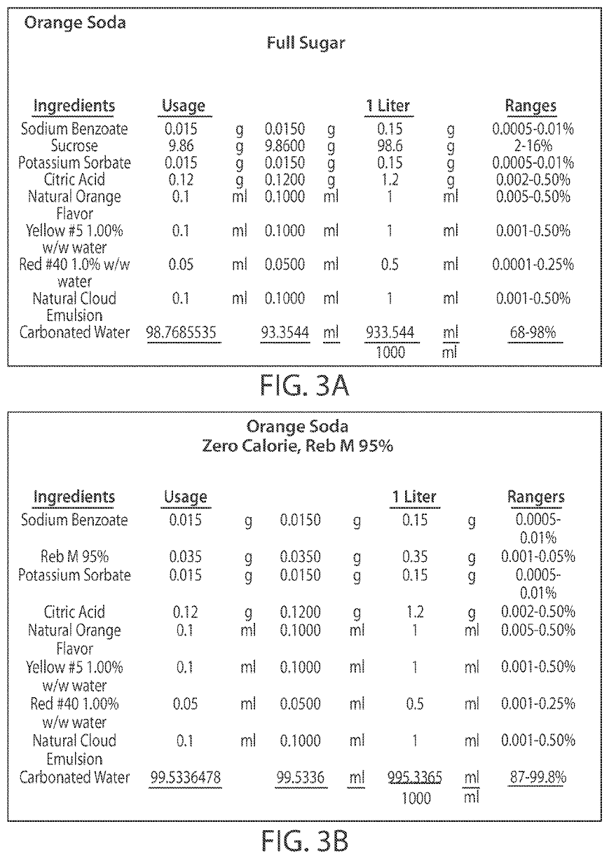

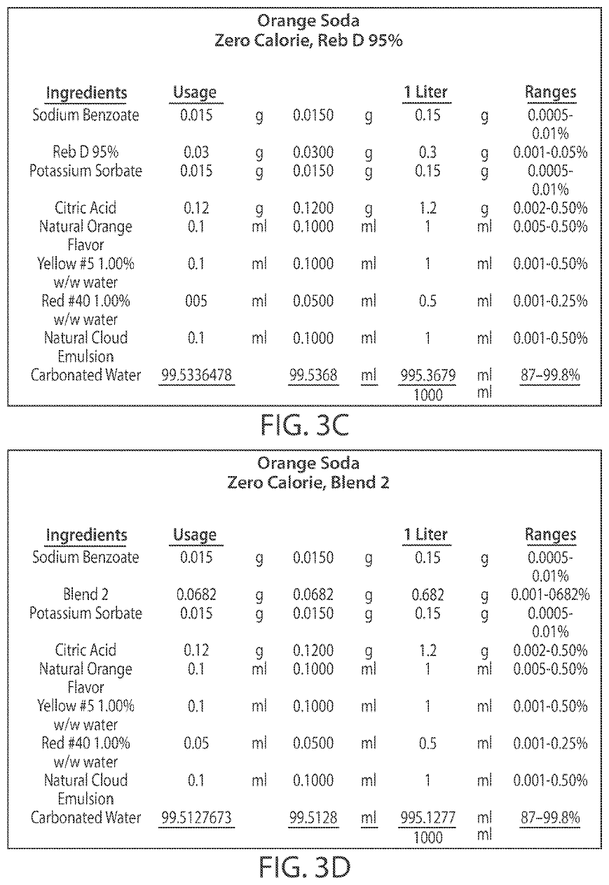

For a carbonated orange drink (See, e.g., FIGS. 3A-3E) the concentrations can be: Orange concentrate (35%), citric acid (0.35%), ascorbic acid (0.05%), orange red color (0.01%), and orange flavor (0.20%), with a rebaudioside blend present at approximately (0.003%). Each rebaudioside composition (e.g., 0.03%) is blended and can dissolve completely in water (up to 100%) and can be pasteurized. The preservatives sodium citrate and/or sodium 15 benzoate can be used according to usage as known by those skilled in the art to maintain shelf-life.

It should also be noted that the protein sweeteners a formulation as provided herein when use as a sweetener for a protein composition? can slightly increase the calorific value per unit sweetness of the composition.

Typically, low- or non-caloric sweeteners based on steviol glycosides tend to have bitter and licorice aftertastes, especially rebaudioside A. Characteristics are especially notable at concentrations above about 300 ppm. In food applications, preferred use levels are often in the range from 480 ppm to about 1000 ppm, above the range at which off flavors are noticed. At the same time, as described above, the sweetening taste of the present formulations is optimal, generally, having no bitterness and leaving no unpleasant aftertaste, commonly experienced with other sweeteners.

The inventive formulations provided herein generally are many times sweeter than sucrose and much smaller amounts are needed to produce the same sweetening effect as a given amount of sugar. Therefore, the caloric intake of the consumer is vastly reduced, making the calorie-add essentially negligible. The formulations provided herein are thus also suitable for incorporation into dietetic foods or diabetic foods.

The characteristics of attribute testing are provided Table 6 below. Table 7 below shows data from sensory testing at various time points.

TABLE 6
AROMA/FLAVOR

| | |
|---|---|
| Total Aroma | The total aroma intensity of the sample. |
| Total Flavor | The total flavor intensity of the sample, including the |
| | basic tastes. |
| Total Oil | The intensity of aroma/flavor of any type of oil, |
| | including oxidized oil. |
| Oxidized Oil | The intensity of aroma/flavor of oxidized oil, described |
| | as old oil that has undergone oxidation, characterized as |
| | cardboard, beany, painty, or fishy. |
| Total Off | The intensity of aroma/flavor of believed to not intended |
| Aroma/Flavor | in the product, includes oxidized oil and other off notes. |
| | The nature of the off note is to be described. |
| Mayonnaise/ | The intensity of the aroma/flavor associated with |
| Dairy | mayonnaise or dairy product. |
| Vinegar | The intensity of the aroma/flavor of white vinegar or |
| | acetic acid. |
| Onion/Garlic/ | The intensity of aroma/flavor associated with onion, |
| Herb | garlic, and all dried and fresh green herbs. |
| Sour | One of the four basic tastes, perceived primarily on the |
| | sides of the tongue; common to acids. |
| Salty | One of the four basic tastes, perceived primarily on the |
| | sides of the tongue; common to sodium chloride (table |
| | salt). |

TABLE 7
FEELING FACTORS

| | |
|---|---|
| Pungent | The amount of burning or irritation of the nasal |
| | cavity produced by smelling the sample, such as |
| | with horseradish. |

TEXTURE

| | |
|---|---|
| Viscosity by Mouth | The degree of thickness of the sample as perceived |
| | when manipulated in the mouth. |
| Oily Mouthcoating | The amount of coating perceived on the soft tissues |
| | of the mouth |

AFTERTASTE

| | |
|---|---|
| Total Aftertaste | The total aftertaste intensity of the sample. |

TABLE 8
SCALE REFERENCES

VALUE

APPEARANCE

| | | |
|---|---|---|
| Color | 0.0 | White (paper) |
| | 7.5 | Manila Folder |

AROMA\FLAVOR

| | | |
|---|---|---|
| Eggy | 8.0/6.0 | Chopped Hard Boiled Eggs |
| Vinegar Aroma | 6.5 | 100% Heinz Distilled Vinegar solution |
| Vinegar Flavor | 4.0 | 2% Heinz Distilled Vinegar solution |
| Sweet | 2.0 | 2.0% Sucrose in Water |
| | 5.0 | 5.0% Sucrose in Water |
| Sour | 2.0 | 0.025% Citric Acid in Water |
| | 5.0 | 0.04% Citric Acid in Water |
| Salty | 2.0 | 0.2% Sodium Chloride in Water |
| | 5.0 | 0.5% Sodium Chloride in Water |

MOUTHFEEL FACTORS

| | | |
|---|---|---|
| Pungent (aroma) | 8.0 | 100% Heinz Distilled Vinegar solution |

TEXTURE

| | | |
|---|---|---|
| Viscosity by Mouth | 8.0 | 50:50 mix of Lucerne Heavy Cream and |
| | | Kraft Mayonnaise |
| | 11.0 | Kraft Mayonnaise |
| Oily Mouthfeel | 8.0 | Kraft Mayonnaise |

### LITERATURE CITED AND INCORPORATED BY REFERENCE

1. Burkholder W. J. et al., Foods and techniques for managing obesity in companion animals, J AM VET MED ASSOC. (1998 Mar. 1); 212(5):658-62.
2. Chang S. S. et al., Stability Studies of Stevioside and Rebaudioside A in Carbonated Beverages, J. AGRIC. FOOD CHEM. (1983) 31: 409-12.
3. Chey, P., & Phillips, C. The rate of intake of sweet, salty and bitter concentrates by dairy cows, ANIMAL SCIENCE (1999) 68(4), 731-40.
4. Frederick, G., et al., Masking the taste of rapeseed meal in dairy compound food, ANIMAL PRODUCTION 46 (1988): 518 (abstract)
5. Fu, M., et al., Dietary bitterness reduces feed intake in piglets with or without gastrointestinal discomfort conditioning, CHEMICAL SENSES (40) (2015) 273-74.
6. Gomez, M. L. M., et al., Sensory Evaluation of Sherry Vinegar Traditional Compared to Accelerated Aging with Oak Chips, J. FOOD SCIENCE 71(3) S238-S242 (2006).
7. Hand M. S. et al., Commercial Pet Foods, in SMALL ANIMAL CLINICAL NUTRITION (5th ed. Topeka, KS: Mark Morris Institute publ., (2010)), pp 160-64, 210-221.
8. Hersleth M., et al., Perception of Bread: A Comparison of Consumers and Trained Assessors, J. FOOD SCIENCE 70(2) S95-101 (2005).
9. Linder D. E., et al., Status of selected nutrients in obese dogs undergoing caloric restriction, BMC VET RES. 2013 Oct. 24;9:219
10. Liu, J., et al., Sensory and Chemical Analyses of Oyster Mushrooms (Pleurotus Sajor-Caju) Harvested from Different Substrates, J. FOOD SCIENCE 70(9): S586-S592 (2005).
11. MANUAL ON DESCRIPTIVE ANALYSIS TESTING, FOR SENSORY EVALUATION, (edit. Hootman, R. C., 1992) ASTM Manual Series: MNL (13) pp 1-51 (publ. ASTM).
12. Matta, Z., et al., Consumer and Descriptive Sensory Analysis of Black Walnut Syrup, J. FOOD SCIENCE 70(9): S610-S613 (2005).
13. O'Brien R. D., FATS AND OILS, FORMULATING AND PROCESSING FOR APPLICATIONS, (publ. CRC Press)(2^{nd} edit. 2003).
14. Omega Pure, FOOD PRODUCT APPLICATIONS, Product Insert (2006).
15. Sidel & Stone, Sensory Science: Methodology in, HANDBOOK OF FOOD SCIENCE, TECHNOLOGY AND ENGINEERING VOL. 2, pp. 57-3 through 57-24 (edit. Hui, Y. H., 2005).
16. STANDARD GUIDE FOR SENSORY EVALUATION METHODS TO DETERMINE THE SENSORY SHELF-LIFE OF CONSUMER PRODUCTS, (publ. ASTM Int'l) publication E2454-05; pp. 1-9 (2005).
17. Weissbach and Weissbach, METHODS FOR PLANT MOLECULAR BIOLOGY, (Academic Press, (1989)).

Although the foregoing disclosure has been described in some detail by way of illustration and example for purposes of understanding, it will be apparent to those skilled in the art that certain changes and modifications may be practiced. Therefore, the description and examples should not be construed as limiting the scope of the disclosure, which is delineated by the appended claims.

Accordingly, it is to be understood that embodiments providing for an improved composition of rebaudiosides for utilization in food/feed products should not be limited to the specific examples. These examples are illustrative of the general applicability of the current disclosure to a vast range of food/feed items. With the inclusion of the rebaudioside sweetener formulations provided herein, these items can be made with the same or better sensory qualities while enhancing the nutritional quality of the food produced for human or animal consumption.

Moreover, the examples provided herein are merely illustrative of the application of the principles of the disclosure. It will be evident from the foregoing description that changes in the form, methods of use, and applications of the elements of the disclosed rebaudioside formulations could be used for applications not limited to human consumption, such as referred to above, for the development of feed for use both for companion animals as well as in animal production industries generally including but not limited to: beef production; poultry production; pork production, and/or aquaculture. These variant uses may be resorted to without departing from the spirit of the disclosure, or the scope of the appended claims.

What is claimed is:

1. A steviol glycoside formulation consisting essentially of 40-60wt. % rebaudioside A (Reb A), 15-30 wt. % rebaudioside E (Reb E), 10-17 wt. % rebaudioside D (Reb D), and 5-10 wt. % rebaudioside M (Reb M).

2. A steviol glycoside formulation consisting essentially of 40-60 wt. % rebaudioside A (Reb A), 15-30 wt. % rebaudioside E (Reb E), 10-17 wt. % rebaudioside D (Reb D), 5-10 wt. % rebaudioside M (Reb M), and 2-8 wt. % rebaudioside I (Reb I).

3. The steviol glycoside formulation of claim 1, wherein Reb A is present in a concentration of 300-600 ppm, Reb E is present in a concentration of 50-200 ppm, Reb D is present in a concentration of 50-200 ppm, Reb M is present in a concentration of 200-500 ppm.

4. The steviol glycoside formulation of claim 2, wherein Reb A is present in a concentration of 200-500 ppm, Reb E is present in a concentration of 50-300 ppm, Reb D is present in a concentration of 50-300 ppm, Reb M is present in a concentration of 5-100 ppm, and Reb I is present in a concentration of 5-50 ppm.

5. A steviol glycoside formulation consisting essentially of rebaudioside A (Reb A), rebaudioside E (Reb E), rebaudioside D (Reb D), and rebaudioside M (Reb M), wherein Reb A is present in an amount of 300-600 ppm; Reb E is present in an amount of from 50-250 ppm; Reb D is present in an amount of 10-200 ppm; and/or Reb M is present in an amount of 10-150 ppm.

6. The steviol glycoside formulation of claim 5, further comprising rebaudioside I (Reb I) in an amount of 1-50 ppm.

7. A steviol glycoside formulation consisting essentially of 500 ppm Reb A, 350 ppm Reb M, 100 ppm Reb D, and 100 ppm Reb E.

8. A steviol glycoside formulation consisting essentially of 373 ppm Reb A, 48 ppm Reb M, 100 ppm Reb D, 131 ppm Reb E, and 30 ppm Reb I.

9. The steviol glycoside formulation of any one of claims 1-8, wherein at least one rebaudioside is made by a genetically modified microbe.

10. An orally consumable product comprising the steviol glycoside formulation of any one of claims 1-9, or the sweetener of any one of claims 43-54.

11. The orally consumable product of claim 10, wherein the orally consumable product is selected from the group consisting of a food composition, a beverage product, a dietary supplement, a nutraceutical, an edible gel mix, an edible gel composition, a pharmaceutical composition, a dental and oral hygiene composition, and an animal feed.

12. The orally consumable product of claim 11, wherein the orally consumable product is a dental and oral hygiene composition.

13. The orally consumable product of claim 12, wherein the dental and oral hygiene composition is a toothpaste.

14. The orally consumable product of claim 12 or claim 13, wherein the steviol glycoside formulation is present in a concentration of 50-800 ppm.

15. The orally consumable product of any one of claims 12-14, wherein the steviol glycoside formulation is present in the range of 0.0003% to 1.0% by weight of the total weight of the orally consumable product.

16. The orally consumable product of claim 11, wherein the orally consumable product is a pharmaceutical composition.

17. The orally consumable product of claim 16, wherein the steviol glycoside formulation is present in a concentration of 50-800 ppm.

18. The orally consumable product claim 16 or claim 17, wherein the steviol glycoside formulation is present in the range of 0.0004% to 1.25% by weight of the total weight of the orally consumable product.

19. The orally consumable product of claim 11, wherein the orally consumable product is a beverage.

20. The orally consumable product of claim 19, wherein the beverage is a carbonated or non-carbonated beverage.

21. The orally consumable product of claim 20, wherein the beverage is selected from the group consisting of a soft drink, a fountain beverage, a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink.

22. The orally consumable product of any one of claims 19-21, wherein the steviol glycoside formulation is present in a concentration of 65-800 ppm.

23. The orally consumable product of any one of claims 19-22, wherein the steviol glycoside formulation is present in the range of 0.0005% to 1.0% by weight of the total weight of the orally consumable product.

24. The orally consumable product of claim 11, wherein the orally consumable product is a food composition.

25. The orally consumable product of claim 24, wherein the food composition is selected from the group consisting of spreads, margarines, sports products, nutrition bars, infant formulas, mayonnaise, confectionary composition, a condiment, a chewing gum, a cereal composition, a baked good, a dairy product, and a tabletop sweetener composition.

26. The orally consumable product of claim 25, wherein the food composition is a yogurt.

27. The orally consumable product of claim 24 or claim 25, wherein the food composition is frozen.

28. The orally consumable product of claim 27, wherein the food composition is ice cream.

29. The orally consumable product of any one of claims 24-28, wherein the steviol glycoside formulation is present in a concentration of 50-700 ppm.

30. The orally consumable product of any one of claims 24-29, wherein the steviol glycoside formulation is present in the range of 0.0005% to 1.0% by weight of the total weight of the orally consumable product.

31. The orally consumable product of any one of claims 24-30, further comprising a component selected from the group consisting of sucrose, aroma compounds, flavoring compounds and mixtures thereof.

32. The orally consumable product of claim 31, further comprising tocopherols in an amount of at least 5 ppm.

33. The orally consumable product of any one of claims 24-32, wherein further comprising at least one stabilizing agent selected from the group consisting of citric acid, sodium benzoate, t-butyl hydroquinone, ascorbyl palmitate, propyl gallate, and combinations thereof.

34. The orally consumable product of any one of claims 24-33, further comprising a moisture containing ingredient.

35. The orally consumable product of claim 34, wherein the moisture ingredient is an emulsion.

36. The orally consumable product of any one of claims 24-35, further comprising a chelating agent.

37. The orally consumable product of claim 11, wherein the orally consumable product is an animal feed product for livestock, companion animals and/or aquaculture.

38. The orally consumable product of claim 37, wherein the livestock is cattle, swine and/or poultry.

39. The orally consumable product of claim 37 or claim 38, wherein the steviol glycoside formulation is present in a concentration of 50-800 ppm.

40. The orally consumable product of any one of claims 37-39, further comprising a hydrocolloid or erythritol.

41. A composition in any one of the figures.

42. A method for creating or enhancing a sweetening effect in an orally consumable product comprising adding an amount of the steviol glycoside formulation of any one of claims 1-9 or the sweetener of any one of claims 43-54 sufficient to produce the desired degree of sweetness to the orally consumable product.

43. A sweetener comprising rebaudioside I (Reb I) produced by a reaction mixture comprising a steviol glycoside; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of any one of SEQ ID NOs: 1-4.

44. The sweetener of claim 43, wherein the reaction mixture further comprises a sucrose synthase comprising the amino acid sequence of SEQ ID NO: 8.

45. The sweetener of claim 43 or claim 44, wherein the steviol glycoside is rebaudioside A.

46. The sweetener of any one of claims 43-45, further comprising one or more steviol glycoside selected from the group consisting of: rebaudioside E (Reb E), rebaudioside A (Reb A), rebaudioside M (Reb M), and rebaudioside D (Reb D).

47. The sweetener of claim 46, further comprising Reb E, Reb A, Reb M, and Reb D.

48. The sweetener of claim 46 or claim 47, wherein the Reb E is produced by a reaction mixture comprising stevioside, rebaudioside KA, or rubusoside; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of any one of SEQ ID NOs: 1, 5, and 7.

49. The sweetener of any one of claims 46-48, wherein the Reb A is produced by a reaction mixture comprising stevioside or rebaudioside D; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of SEQ ID NO: 1.

50. The sweetener of any one of claims 46-49, wherein the Reb M is produced by a reaction mixture comprising stevioside or rebaudioside D; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of SEQ ID NO: 1 or SEQ ID NO: 7.

51. The sweetener of any one of claims 46-50 wherein the Reb D is produced by a reaction mixture comprising rebaudioside A or rebaudioside E; a substrate selected from the group consisting of sucrose, uridine diphosphate (UDP), and uridine diphosphate-glucose (UDP-glucose); and an uridine dipospho glycosyltransferases (UDP-glycosyltransferase) comprising the amino acid sequence of any one of SEQ ID NOs: 1, 5, and 7.

52. The sweetener of any one of claims 48-51, wherein the reaction mixture further comprises a sucrose synthase comprising the amino acid sequence of SEQ ID NO: 8.

53. The sweetener of any one of claims 47-52, comprising 40-60 wt. % Reb A, 15-30 wt. % Reb E, 10-17 wt. % Reb D, 5-10 wt. % Reb M, and 2-8 wt. % Reb I.

54. The sweetener of any one of claims 47-53, wherein Reb A is present in a concentration of 200-500 ppm, Reb E is present in a concentration of 50-300 ppm, Reb D is present in a concentration of 50-300 ppm, Reb M is present in a concentration of 5-100 ppm, and Reb I is present in a concentration of 5-50 ppm.


SEQUENCE LISTING (30 sequences)

SEQ ID NO: 1 - protein, 458 aa, Artificial Sequence
  Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - protein, 458 aa, Artificial Sequence
  Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQIAKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 3 - protein, 458 aa, Artificial Sequence
  Synthetic
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 4 - protein, 475 aa, Artificial Sequence
  Synthetic
MNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLYKDDSGYSSSYAAAAGMENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYS

SEQ ID NO: 5 - protein, 462 aa, Artificial Sequence
  Synthetic
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 6 - protein, 462 aa, Artificial Sequence
  Synthetic
MGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTK

SEQ ID NO: 7 - protein, 459 aa, Artificial Sequence
  Synthetic
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 8 - protein, 808 aa, Artificial Sequence
  Synthetic
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 9 - protein, 1270 aa, Artificial Sequence
  Synthetic
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSLGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDDWT

SEQ ID NO: 10 - protein, 1272 aa, Artificial Sequence
  Synthetic
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKDGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 11 - protein, 1269 aa, Artificial Sequence
  Synthetic
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKAGSGANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 12 - protein, 839 aa, Artificial Sequence
  Synthetic
MTQLDVESLIQELTLNEKVQLLSGSDFWHTTPVRRLGIPKMRLSDGPNGVRGTKFFNGVPTACFPCGTGLGATFDKELLKEAGSLMADEAKAKAASVVLGPTANIARGPNGGRGFESFGEDPVVNGLSSAAMINGLQGKYIAATMKHYVCNDLEMDRNCIDAQVSHRALREVYLLPFQIAVRDANPRAIMTAYNKANGEHVSQSKFLLDEVLRKEWGWDGLLMSDWFGVYDAKSSITNGLDLEMPGPPQCRVHSATDHAINSGEIHINDVDERVRSLLSLINYCHQSGVTEEDPETSDNNTPETIEKLRKISRESIVLLKDDDRNRSILPLKKSDKIAVIGNNAKQAAYCGGGSASVLSYHTTTPFDSIKSRLEDSNTPAYTIGADAYKNLPPLGPQMTDSDGKPGFDAKFFVGSPTSKDRKLIDHFQLTNSQVFLVDYYNEQIPENKEFYVDVEGQFIPEEDGTYNFGLTVFGTGRLFVDDKLVSDSSQNQTPGDSFFGLAAQEVIGSIHLVKGKAYKIKVLYGSSVTRTYEIAASVAFEGGAFTFGAAKQRNEDEEIARAVEIAKANDKVVLCIGLNQDFESEGFDRPDIKIPGATNKMVSAVLKANPNTVIVNQTGTPVEMPWASDAPVILQAWFGGSEAGTAIADVLFGDYNPSGKLTVTFPLRFEDNPAYLNFQSNKQACWYGEDVYVGYRYYETIDRPVLFPFGHGLSFTEFDFTDMFVRLEEENLEVEVVVRNTGKYDGAEVVQLYVAPVSPSLKRPIKELKEYAKIFLASGEAKTVHLSVPIKYATSFFDEYQKKWCSEKGEYTILLGSSSADIKVSQSITLEKTTFWKGL

SEQ ID NO: 13 - protein, 346 aa, Artificial Sequence
  Synthetic
MKSQLIFMALASLVASAPLEHQQQHHKHEKRAVVTQTVTVAAGQTAAAGSAQAVVTSSAAPASVASSAAASASSSSSSYTSGASGDLSSFKDGTIKCSEFPSGDGVVSVSWLGFGGWSSIMNLQGGTSESCENGYYCSYACEAGYSKTQWPSNQPSDGRSVGGLLCKDGLLYRSNTAFDTLCVPGKGTASVENNVSKGISICRTDYPGSENMCVPTWVDAGNSNTLTVVDEDNYYEWQGLKTSAQYYVNNAGVSVEDGCIWGDESSGVGNWAPLVLGAGSTGGLTYLSLIPNPNNKKAPNFNVKIVATDGSSINGDCKYENGIFVGSSTDGCTVTVTSGSAKLVFY

SEQ ID NO: 14 - protein, 348 aa, Artificial Sequence
  Synthetic
MQVKSIVNLLLACSLAVARPLEHAHHQHDKRGVVVVTKTIVVDGSTVEATAAAQVQEHAETFAESTPSAVVSSSSAPSSASSASAPASSGSFSAGTKGVTYSPYQAGGGCKTAEEVASDLSQLTGYEIIRLYGVDCNQVENVFKAKAPGQKLFLGIFFVDAIESGVSAIASAVKSYGSWDDVHTVSVGNELVNNGEATVSQIGQYVSTAKSALRSAGFTGPVLSVDTFIAVINNPGLCDFADEYVAVNAHAFFDGGIAASGAGDWAAEQIQRVSSACGGKDVLIVESGWPSKGDTNGAAVPSKSNQQAAVQSLGQKIGSSCIAFNAFNDYWKADGPFNAEKYWGILDS

SEQ ID NO: 15 - protein, 464 aa, Artificial Sequence
  Synthetic
MLSTILNIFILLLFIQASLQAPIPVVTKYVTEGIAVVTETNVRVVTKTIPIVQVLISDGATYTHTLTTVSTAEENGNFQPITTTSIVNKEVVVPTSVTPNTQQTRPTQVDTTQNNADTPAAPTPSPTTSSNNGVFTTYSTTRSVVTSVVVVGPDGSPIENTGQTANPTTTAPTTSTTAARTTSSTSTTPTASSTPGGNHPRSIVYSPYSDSSQCKDATTIETDLEFIASKGISAVRIYGNDCNYLTVVLPKCASLGLKVNQGFWIGPSGVDSIDDAVQEFIQAVNGNNGFNWDLFELITVGNEAISAGYVSASSLISKIKEVSSILSSAGYTGPITTAEPPNVYEDYGDLCSTDVMSIVGVNAHSYFNTLFAASDSGSFVKSQIEVVQKACSRSDITIIETGYPSQGATNGKNVPSKENQKTAIFSIFEVVGTDVTILSTYDDLWKDPGPYGIEQFFGAIDLFS

SEQ ID NO: 16 - DNA, 1377 bp, Artificial Sequence
  Synthetic
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttataa

SEQ ID NO: 17 - DNA, 1377 bp, Artificial Sequence
  Synthetic
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattgcgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttataa

SEQ ID NO: 18 - DNA, 1377 bp, Artificial Sequence
  Synthetic
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgtaa

SEQ ID NO: 19 - DNA, 1428 bp, Artificial Sequence
  Synthetic
atgaactggcaaatcctgaaagaaatcctgggtaaaatgatcaaacaaaccaaagcgtcgtcgggcgttatctggaactccttcaaagaactggaagaatcagaactggaaaccgttattcgcgaaatcccggctccgtcgttcctgattccgctgccgaaacatctgaccgcgagcagcagcagcctgctggatcacgaccgtacggtctttcagtggctggatcagcaaccgccgtcatcggtgctgtatgtttcattcggtagcacctctgaagtcgatgaaaaagactttctggaaatcgctcgcggcctggtggatagtaaacagtccttcctgtgggtggttcgtccgggttttgtgaaaggcagcacgtgggttgaaccgctgccggatggcttcctgggtgaacgcggccgtattgtcaaatgggtgccgcagcaagaagtgctggcacatggtgctatcggcgcgttttggacccactctggttggaacagtacgctggaatccgtttgcgaaggtgtcccgatgattttcagcgattttggcctggaccagccgctgaatgcccgctatatgtctgatgttctgaaagtcggtgtgtacctggaaaacggttgggaacgtggcgaaattgcgaatgccatccgtcgcgttatggtcgatgaagaaggcgaatacattcgccagaacgctcgtgtcctgaaacaaaaagcggacgtgagcctgatgaaaggcggtagctcttatgaatcactggaatcgctggttagctacatcagttccctgtacaaagatgacagcggttatagcagcagctatgcggcggcggcgggtatggaaaataaaaccgaaaccacggtgcgtcgccgtcgccgtattatcctgttcccggttccgtttcagggtcatattaacccgatcctgcaactggcgaatgttctgtattcaaaaggcttttcgatcaccatcttccatacgaacttcaacaaaccgaaaaccagtaactacccgcactttacgttccgctttattctggataacgacccgcaggatgaacgtatctccaatctgccgacccacggcccgctggccggtatgcgcattccgattatcaatgaacacggtgcagatgaactgcgccgtgaactggaactgctgatgctggccagtgaagaagatgaagaagtgtcctgtctgatcaccgacgcactgtggtatttcgcccagagcgttgcagattctctgaacctgcgccgtctggtcctgatgacgtcatcgctgttcaattttcatgcgcacgtttctctgccgcaatttgatgaactgggctacctggacccggatgacaaaacccgtctggaagaacaagccagtggttttccgatgctgaaagtcaaagacattaaatccgcctattcgtaa

SEQ ID NO: 20 - DNA, 1389 bp, Artificial Sequence
  Synthetic
atggattcgggttactcttcctcctatgcggcggctgcgggtatgcacgttgttatctgtccgtggctggcttttggtcacctgctgccgtgcctggatctggcacagcgtctggcttcacgcggccatcgtgtcagcttcgtgtctaccccgcgcaatatttcgcgtctgccgccggttcgtccggcactggctccgctggttgcatttgtcgctctgccgctgccgcgcgtggaaggtctgccggatggtgcggaaagtaccaacgacgtgccgcatgatcgcccggacatggttgaactgcaccgtcgtgcattcgatggtctggcagcaccgttttccgaatttctgggtacggcgtgcgccgattgggtgatcgttgacgtctttcatcactgggcggcggcggcggcgctggaacataaagttccgtgtgcaatgatgctgctgggctcagctcacatgattgcgtcgatcgcagaccgtcgcctggaacgtgcagaaaccgaaagtccggctgcggccggccagggtcgcccggcagctgcgccgaccttcgaagtggcccgcatgaaactgattcgtacgaaaggcagctctggtatgagcctggcagaacgctttagtctgaccctgtcccgtagttccctggtggttggtcgcagttgcgttgaatttgaaccggaaaccgtcccgctgctgtccacgctgcgtggtaaaccgatcacctttctgggtctgatgccgccgctgcatgaaggccgtcgcgaagatggtgaagacgcaacggtgcgttggctggatgcacagccggctaaaagcgtcgtgtatgtcgccctgggctctgaagtgccgctgggtgtggaaaaagttcacgaactggcactgggcctggaactggctggcacccgcttcctgtgggcactgcgtaaaccgacgggtgtgagcgatgcggacctgctgccggccggttttgaagaacgtacccgcggccgtggtgttgtcgcaacgcgttgggtcccgcaaatgagcattctggcgcatgccgcagtgggcgcctttctgacccactgtggttggaacagcacgatcgaaggcctgatgtttggtcacccgctgattatgctgccgatcttcggcgatcagggtccgaacgcacgtctgattgaagcgaaaaatgccggcctgcaagttgcgcgcaacgatggcgacggttctttcgaccgtgagggtgtggctgcggccattcgcgcagtggctgttgaagaagaatcatcgaaagtttttcaggcgaaagccaaaaaactgcaagaaatcgtcgcggatatggcctgccacgaacgctacattgatggtttcattcagcaactgcgctcctacaaagactaa

SEQ ID NO: 21 - DNA, 1389 bp, Artificial Sequence
  Synthetic
atgggtagctcgggcatgtccctggcggaacgcttttcgctgacgctgagtcgctcatccctggttgttggtcgcagttgtgttgaatttgaaccggaaaccgttccgctgctgtctacgctgcgcggcaaaccgattaccttcctgggtctgatgccgccgctgcatgaaggccgtcgcgaagatggtgaagacgccacggtgcgttggctggatgctcagccggcgaaatcggtggtttatgtcgcactgggcagcgaagtgccgctgggtgtcgaaaaagtgcacgaactggccctgggcctggaactggcaggcacccgctttctgtgggcactgcgtaaaccgacgggcgttagcgatgctgacctgctgccggcgggtttcgaagaacgcacccgcggccgtggtgtcgtggccacccgttgggtgccgcaaatgtccattctggctcatgcggccgttggcgcatttctgacccactgcggttggaacagcacgatcgaaggcctgatgtttggtcatccgctgattatgctgccgatcttcggcgatcagggtccgaacgcacgcctgatcgaagccaaaaatgcaggcctgcaagttgcgcgtaacgatggcgacggtagctttgaccgcgaaggtgtcgcagctgcgattcgtgctgtggcggttgaagaagaaagcagcaaagtcttccaggccaaagcgaaaaaactgcaagaaatcgtggctgatatggcgtgtcatgaacgctatattgacggctttatccagcaactgcgttcttacaaagatgacagtggctatagttcctcatacgccgcagctgcgggtatgcatgttgtcatttgcccgtggctggcgtttggtcacctgctgccgtgtctggatctggcacagcgcctggcatctcgcggtcaccgtgtttcgttcgtcagcaccccgcgcaatatcagtcgtctgccgccggttcgtccggcgctggcgccgctggttgcgttcgttgcactgccgctgccgcgtgtggaaggtctgccggatggtgccgaatcgaccaacgacgttccgcatgatcgtccggacatggtcgaactgcatcgtcgcgcctttgatggcctggccgcaccgtttagcgaatttctgggtacggcctgcgcagattgggtcattgtggacgtttttcaccactgggcggcggcggcggcgctggaacataaagtgccgtgtgcgatgatgctgctgggttccgcccacatgattgcttcaatcgcggatcgtcgcctggaacgtgccgaaaccgaaagtccggcggcggcaggccagggtcgtccggcggcggcaccgacctttgaagtggcacgtatgaaactgattcgcacgaaataa

SEQ ID NO: 22 - DNA, 1380 bp, Artificial Sequence
  Synthetic
atggatggtaactcctcctcctcgccgctgcatgtggtcatttgtccgtggctggctctgggtcacctgctgccgtgtctggatattgctgaacgtctggcgtcacgcggccatcgtgtcagttttgtgtccaccccgcgcaacattgcccgtctgccgccgctgcgtccggctgttgcaccgctggttgatttcgtcgcactgccgctgccgcatgttgacggtctgccggagggtgcggaatcgaccaatgatgtgccgtatgacaaatttgaactgcaccgtaaggcgttcgatggtctggcggccccgtttagcgaatttctgcgtgcagcttgcgcagaaggtgcaggttctcgcccggattggctgattgtggacacctttcatcactgggcggcggcggcggcggtggaaaacaaagtgccgtgtgttatgctgctgctgggtgcagcaacggtgatcgctggtttcgcgcgtggtgttagcgaacatgcggcggcggcggtgggtaaagaacgtccggctgcggaagccccgagttttgaaaccgaacgtcgcaagctgatgaccacgcagaatgcctccggcatgaccgtggcagaacgctatttcctgacgctgatgcgtagcgatctggttgccatccgctcttgcgcagaatgggaaccggaaagcgtggcagcactgaccacgctggcaggtaaaccggtggttccgctgggtctgctgccgccgagtccggaaggcggtcgtggcgtttccaaagaagatgctgcggtccgttggctggacgcacagccggcaaagtcagtcgtgtacgtcgcactgggttcggaagtgccgctgcgtgcggaacaagttcacgaactggcactgggcctggaactgagcggtgctcgctttctgtgggcgctgcgtaaaccgaccgatgcaccggacgccgcagtgctgccgccgggtttcgaagaacgtacccgcggccgtggtctggttgtcacgggttgggtgccgcagattggcgttctggctcatggtgcggtggctgcgtttctgacccactgtggctggaactctacgatcgaaggcctgctgttcggtcatccgctgattatgctgccgatcagctctgatcagggtccgaatgcgcgcctgatggaaggccgtaaagtcggtatgcaagtgccgcgtgatgaatcagacggctcgtttcgtcgcgaagatgttgccgcaaccgtccgcgccgtggcagttgaagaagacggtcgtcgcgtcttcacggctaacgcgaaaaagatgcaagaaattgtggccgatggcgcatgccacgaacgttgtattgacggttttatccagcaactgcgcagttacaaggcgtaa

SEQ ID NO: 23 - DNA, 2427 bp, Artificial Sequence
  Synthetic
atggcaaacgctgaacgtatgattacccgtgtccactcccaacgcgaacgcctgaacgaaaccctggtgtcggaacgcaacgaagttctggcactgctgagccgtgtggaagctaagggcaaaggtattctgcagcaaaaccagattatcgcggaatttgaagccctgccggaacaaacccgcaaaaagctggaaggcggtccgtttttcgatctgctgaaatctacgcaggaagcgatcgttctgccgccgtgggtcgcactggcagtgcgtccgcgtccgggcgtttgggaatatctgcgtgtcaacctgcatgcactggtggttgaagaactgcagccggctgaatttctgcacttcaaggaagaactggttgacggcgtcaaaaacggtaattttaccctggaactggattttgaaccgttcaatgccagtatcccgcgtccgacgctgcataaatatattggcaacggtgtggactttctgaatcgccatctgagcgcaaagctgttccacgataaagaatctctgctgccgctgctgaaattcctgcgtctgcatagtcaccagggcaagaacctgatgctgtccgaaaaaattcagaacctgaataccctgcaacacacgctgcgcaaggcggaagaatacctggccgaactgaaaagtgaaaccctgtacgaagaattcgaagcaaagttcgaagaaattggcctggaacgtggctggggtgacaatgctgaacgtgttctggatatgatccgtctgctgctggacctgctggaagcaccggacccgtgcaccctggaaacgtttctgggtcgcgtgccgatggttttcaacgtcgtgattctgtccccgcatggctattttgcacaggacaatgtgctgggttacccggataccggcggtcaggttgtctatattctggatcaagttcgtgcgctggaaattgaaatgctgcagcgcatcaagcagcaaggcctgaacatcaaaccgcgtattctgatcctgacccgtctgctgccggatgcagttggtaccacgtgcggtgaacgtctggaacgcgtctatgacagcgaatactgtgatattctgcgtgtcccgtttcgcaccgaaaagggtattgtgcgtaaatggatcagtcgcttcgaagtttggccgtatctggaaacctacacggaagatgcggccgtggaactgtccaaggaactgaatggcaaaccggacctgattatcggcaactatagcgatggtaatctggtcgcatctctgctggctcataaactgggtgtgacccagtgcacgattgcacacgctctggaaaagaccaaatatccggattcagacatctactggaaaaagctggatgacaaatatcatttttcgtgtcagttcaccgcggacatttttgccatgaaccacacggattttattatcaccagtacgttccaggaaatcgcgggctccaaagaaaccgtgggtcaatacgaatcacataccgccttcacgctgccgggcctgtatcgtgtggttcacggtatcgatgtttttgacccgaaattcaatattgtcagtccgggcgcggatatgtccatctattttccgtacaccgaagaaaagcgtcgcctgacgaaattccattcagaaattgaagaactgctgtactcggacgtggaaaacaaggaacacctgtgtgttctgaaagataaaaagaaaccgatcctgtttaccatggcccgtctggatcgcgtgaagaatctgtcaggcctggttgaatggtatggtaaaaacacgcgtctgcgcgaactggcaaatctggtcgtggttggcggtgaccgtcgcaaggaatcgaaagataacgaagaaaaggctgaaatgaagaaaatgtacgatctgatcgaagaatacaagctgaacggccagtttcgttggatcagctctcaaatggaccgtgtgcgcaatggcgaactgtatcgctacatttgcgataccaagggtgcgtttgttcagccggcactgtacgaagctttcggcctgaccgtcgtggaagccatgacgtgcggtctgccgacctttgcgacgtgtaaaggcggtccggccgaaattatcgtgcatggcaaatctggtttccatatcgatccgtatcacggtgatcaggcagctgacaccctggcggatttctttacgaagtgtaaagaagacccgtcacactgggatgaaatttcgaagggcggtctgcaacgtatcgaagaaaaatatacctggcagatttacagccaacgcctgctgaccctgacgggcgtctacggtttttggaaacatgtgtctaatctggatcgcctggaagcccgtcgctatctggaaatgttttacgcactgaagtatcgcccgctggcacaagccgttccgctggcacaggacgactaa

SEQ ID NO: 24 - DNA, 3807 bp, Artificial Sequence
  Synthetic
atggagaataagacagaaacaaccgtaagacggaggcggaggattatcttgttccctgtaccatttcagggccatattaatccgatcctccaattagcaaacgtcctctactccaagggattttcaataacaatcttccatactaactttaacaagcctaaaacgagtaattatcctcactttacattcaggttcattctagacaacgaccctcaggatgagcgtatctcaaatttacctacgcatggccccttggcaggtatgcgaataccaataatcaatgagcatggagccgatgaactccgtcgcgagttagagcttctcatgctcgcaagtgaggaagacgaggaagtttcgtgcctaataactgatgcgctttggtacttcgcccaatcagtcgcagactcactgaatctacgccgtttggtccttatgacaagttcattattcaactttcacgcacatgtatcactgccgcaatttgacgagttgggttacctggacccggatgacaaaacgcgattggaggaacaagcgtcgggcttccccatgctgaaagtcaaagatattaagagcgcttatagtaattggcaaattctgaaagaaattctcggaaaaatgataaagcaaaccaaagcgtcctctggagtaatctggaactccttcaaggagttagaggaatctgaacttgaaacggtcatcagagaaatccccgctccctcgttcttaattccactacccaagcaccttactgcaagtagcagttccctcctagatcatgaccgaaccgtgtttcagtggctggatcagcaacccccgtcgtcagttctatatgtaagctttgggagtacttcggaagtggatgaaaaggacttcttagagattgcgcgagggctcgtggatagcaaacagagcttcctgtgggtagtgagaccgggattcgttaagggctcgacgtgggtcgagccgttgccagatggttttctaggggagagagggagaatcgtgaaatgggttccacagcaagaggttttggctcacggagctataggggccttttggacccactctggttggaattctactcttgaaagtgtctgtgaaggcgttccaatgatattttctgattttgggcttgaccagcctctaaacgctcgctatatgtctgatgtgttgaaggttggcgtgtacctggagaatggttgggaaaggggggaaattgccaacgccatacgccgggtaatggtggacgaggaaggtgagtacatacgtcagaacgctcgggttttaaaacaaaaagcggacgtcagccttatgaagggaggtagctcctatgaatccctagaatccttggtaagctatatatcttcgttaggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 25 - DNA, 3819 bp, Artificial Sequence
  Synthetic
atggattcgggttactcttcctcctatgcggcggctgcgggtatgcacgttgttatctgtccgtggctggcttttggtcacctgctgccgtgcctggatctggcacagcgtctggcttcacgcggccatcgtgtcagcttcgtgtctaccccgcgcaatatttcgcgtctgccgccggttcgtccggcactggctccgctggttgcatttgtcgctctgccgctgccgcgcgtggaaggtctgccggatggtgcggaaagtaccaacgacgtgccgcatgatcgcccggacatggttgaactgcaccgtcgtgcattcgatggtctggcagcaccgttttccgaatttctgggtacggcgtgcgccgattgggtgatcgttgacgtctttcatcactgggcggcggcggcggcgctggaacataaagttccgtgtgcaatgatgctgctgggctcagctcacatgattgcgtcgatcgcagaccgtcgcctggaacgtgcagaaaccgaaagtccggctgcggccggccagggtcgcccggcagctgcgccgaccttcgaagtggcccgcatgaaactgattcgtacgaaaggcagctctggtatgagcctggcagaacgctttagtctgaccctgtcccgtagttccctggtggttggtcgcagttgcgttgaatttgaaccggaaaccgtcccgctgctgtccacgctgcgtggtaaaccgatcacctttctgggtctgatgccgccgctgcatgaaggccgtcgcgaagatggtgaagacgcaacggtgcgttggctggatgcacagccggctaaaagcgtcgtgtatgtcgccctgggctctgaagtgccgctgggtgtggaaaaagttcacgaactggcactgggcctggaactggctggcacccgcttcctgtgggcactgcgtaaaccgacgggtgtgagcgatgcggacctgctgccggccggttttgaagaacgtacccgcggccgtggtgttgtcgcaacgcgttgggtcccgcaaatgagcattctggcgcatgccgcagtgggcgcctttctgacccactgtggttggaacagcacgatcgaaggcctgatgtttggtcacccgctgattatgctgccgatcttcggcgatcagggtccgaacgcacgtctgattgaagcgaaaaatgccggcctgcaagttgcgcgcaacgatggcgacggttctttcgaccgtgagggtgtggctgcggccattcgcgcagtggctgttgaagaagaatcatcgaaagtttttcaggcgaaagccaaaaaactgcaagaaatcgtcgcggatatggcctgccacgaacgctacattgatggtttcattcagcaactgcgctcctacaaagacggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattga

SEQ ID NO: 26 - DNA, 3810 bp, Artificial Sequence
  Synthetic
atggatggtaactcctcctcctcgccgctgcatgtggtcatttgtccgtggctggctctgggtcacctgctgccgtgtctggatattgctgaacgtctggcgtcacgcggccatcgtgtcagttttgtgtccaccccgcgcaacattgcccgtctgccgccgctgcgtccggctgttgcaccgctggttgatttcgtcgcactgccgctgccgcatgttgacggtctgccggagggtgcggaatcgaccaatgatgtgccgtatgacaaatttgaactgcaccgtaaggcgttcgatggtctggcggccccgtttagcgaatttctgcgtgcagcttgcgcagaaggtgcaggttctcgcccggattggctgattgtggacacctttcatcactgggcggcggcggcggcggtggaaaacaaagtgccgtgtgttatgctgctgctgggtgcagcaacggtgatcgctggtttcgcgcgtggtgttagcgaacatgcggcggcggcggtgggtaaagaacgtccggctgcggaagccccgagttttgaaaccgaacgtcgcaagctgatgaccacgcagaatgcctccggcatgaccgtggcagaacgctatttcctgacgctgatgcgtagcgatctggttgccatccgctcttgcgcagaatgggaaccggaaagcgtggcagcactgaccacgctggcaggtaaaccggtggttccgctgggtctgctgccgccgagtccggaaggcggtcgtggcgtttccaaagaagatgctgcggtccgttggctggacgcacagccggcaaagtcagtcgtgtacgtcgcactgggttcggaagtgccgctgcgtgcggaacaagttcacgaactggcactgggcctggaactgagcggtgctcgctttctgtgggcgctgcgtaaaccgaccgatgcaccggacgccgcagtgctgccgccgggtttcgaagaacgtacccgcggccgtggtctggttgtcacgggttgggtgccgcagattggcgttctggctcatggtgcggtggctgcgtttctgacccactgtggctggaactctacgatcgaaggcctgctgttcggtcatccgctgattatgctgccgatcagctctgatcagggtccgaatgcgcgcctgatggaaggccgtaaagtcggtatgcaagtgccgcgtgatgaatcagacggctcgtttcgtcgcgaagatgttgccgcaaccgtccgcgccgtggcagttgaagaagacggtcgtcgcgtcttcacggctaacgcgaaaaagatgcaagaaattgtggccgatggcgcatgccacgaacgttgtattgacggttttatccagcaactgcgcagttacaaggcgggttctggtgcaaacgctgaacgtatgataacgcgcgtccacagccaacgtgagcgtttgaacgaaacgcttgtttctgagagaaacgaagtccttgccttgctttccagggttgaagccaaaggtaaaggtattttacaacaaaaccagatcattgctgaattcgaagctttgcctgaacaaacccggaagaaacttgaaggtggtcctttctttgaccttctcaaatccactcaggaagcaattgtgttgccaccatgggttgctctagctgtgaggccaaggcctggtgtttgggaatacttacgagtcaatctccatgctcttgtcgttgaagaactccaacctgctgagtttcttcatttcaaggaagaactcgttgatggagttaagaatggtaatttcactcttgagcttgatttcgagccattcaatgcgtctatccctcgtccaacactccacaaatacattggaaatggtgttgacttccttaaccgtcatttatcggctaagctcttccatgacaaggagagtttgcttccattgcttaagttccttcgtcttcacagccaccagggcaagaacctgatgttgagcgagaagattcagaacctcaacactctgcaacacaccttgaggaaagcagaagagtatctagcagagcttaagtccgaaacactgtatgaagagtttgaggccaagtttgaggagattggtcttgagaggggatggggagacaatgcagagcgtgtccttgacatgatacgtcttcttttggaccttcttgaggcgcctgatccttgcactcttgagacttttcttggaagagtaccaatggtgttcaacgttgtgatcctctctccacatggttactttgctcaggacaatgttcttggttaccctgacactggtggacaggttgtttacattcttgatcaagttcgtgctctggagatagagatgcttcaacgtattaagcaacaaggactcaacattaaaccaaggattctcattctaactcgacttctacctgatgcggtaggaactacatgcggtgaacgtctcgagagagtttatgattctgagtactgtgatattcttcgtgtgcccttcagaacagagaagggtattgttcgcaaatggatctcaaggttcgaagtctggccatatctagagacttacaccgaggatgctgcggttgagctatcgaaagaattgaatggcaagcctgaccttatcattggtaactacagtgatggaaatcttgttgcttctttattggctcacaaacttggtgtcactcagtgtaccattgctcatgctcttgagaaaacaaagtacccggattctgatatctactggaagaagcttgacgacaagtaccatttctcatgccagttcactgcggatattttcgcaatgaaccacactgatttcatcatcactagtactttccaagaaattgctggaagcaaagaaactgttgggcagtatgaaagccacacagcctttactcttcccggattgtatcgagttgttcacgggattgatgtgtttgatcccaagttcaacattgtctctcctggtgctgatatgagcatctacttcccttacacagaggagaagcgtagattgactaagttccactctgagatcgaggagctcctctacagcgatgttgagaacaaagagcacttatgtgtgctcaaggacaagaagaagccgattctcttcacaatggctaggcttgatcgtgtcaagaacttgtcaggtcttgttgagtggtacgggaagaacacccgcttgcgtgagctagctaacttggttgttgttggaggagacaggaggaaagagtcaaaggacaatgaagagaaagcagagatgaagaaaatgtatgatctcattgaggaatacaagctaaacggtcagttcaggtggatctcctctcagatggaccgggtaaggaacggtgagctgtaccggtacatctgtgacaccaagggtgcttttgtccaacctgcattatatgaagcctttgggttaactgttgtggaggctatgacttgtggtttaccgactttcgccacttgcaaaggtggtccagctgagatcattgtgcacggtaaatcgggtttccacattgacccttaccatggtgatcaggctgctgatactcttgctgatttcttcaccaagtgtaaggaggatccatctcactgggatgagatctcaaaaggagggcttcagaggattgaggagaaatacacttggcaaatctattcacagaggctcttgacattgactggtgtgtatggattctggaagcatgtctcgaaccttgaccgtcttgaggctcgccgttaccttgaaatgttctatgcattgaagtatcgcccattggctcaggctgttcctcttgcacaagatgattaa

SEQ ID NO: 27 - DNA, 2520 bp, Artificial Sequence
  Synthetic
atgacccaactggatgtggagagcctgattcaagagctgaccctgaacgaaaaggtgcaactgctgagcggtagcgacttctggcataccaccccggttcgtcgtctgggcatcccgaagatgcgtctgagcgacggtccgaacggcgttcgtggtaccaaattctttaacggtgttccgaccgcgtgcttcccgtgcggtaccggtctgggcgcgacctttgacaaggaactgctgaaagaggcgggtagcctgatggcggatgaagcgaaagcgaaagcggcgagcgtggttctgggtccgaccgcgaacattgcgcgtggtccgaacggtggccgtggcttcgagagcttcggcgaggacccggtggttaacggtctgagcagcgcggcgatgatcaacggcctgcagggcaagtacattgcggcgaccatgaaacactatgtttgcaacgatctggaaatggaccgtaactgcattgacgcgcaagttagccaccgtgcgctgcgtgaggtgtacctgctgccgttccaaatcgcggtgcgtgatgcgaacccgcgtgcgattatgaccgcgtataacaaggcgaacggcgaacacgttagccagagcaaattcctgctggacgaagtgctgcgtaaggagtggggctgggatggtctgctgatgagcgactggtttggtgtttacgatgcgaaaagcagcatcaccaacggcctggacctggagatgccgggtccgccgcagtgccgtgtgcacagcgcgaccgatcacgcgatcaacagcggcgaaatccacattaacgatgttgacgagcgtgtgcgtagcctgctgagcctgattaactactgccaccaaagcggtgttaccgaggaagatccggaaaccagcgacaacaacaccccggaaaccatcgagaagctgcgtaaaatcagccgtgagagcattgtgctgctgaaggacgatgaccgtaaccgtagcattctgccgctgaagaaaagcgacaaaatcgcggttattggtaacaacgcgaaacaagcggcgtattgcggtggcggtagcgcgagcgtgctgagctatcacaccaccaccccgttcgacagcatcaagagccgtctggaagatagcaacaccccggcgtacaccattggtgcggacgcgtataaaaacctgccgccgctgggtccgcaaatgaccgatagcgacggcaagccgggttttgatgcgaaattctttgttggcagcccgaccagcaaggatcgtaaactgatcgaccacttccagctgaccaacagccaagtttttctggtggactactataacgaacagatcccggaaaacaaggagttctacgttgacgtggagggtcaatttattccggaggaagatggcacctataacttcggtctgaccgtgtttggtaccggccgtctgttcgttgatgacaaactggttagcgacagcagccagaaccaaaccccgggcgatagcttctttggtctggcggcgcaggaagtgatcggcagcattcacctggtgaagggtaaagcgtacaagatcaaagttctgtatggcagcagcgtgacccgtacctacgaaattgcggcgagcgttgcgtttgagggcggtgcgttcacctttggtgcggcgaaacagcgtaacgaagacgaggaaatcgcgcgtgcggtggagattgcgaaggcgaacgacaaagtggttctgtgcatcggcctgaaccaagatttcgaaagcgagggttttgatcgtccggacatcaagattccgggcgcgaccaacaaaatggttagcgcggtgctgaaggcgaacccgaacaccgttattgtgaaccagaccggtaccccggttgagatgccgtgggcgagcgatgcgccggtgatcctgcaagcgtggtttggcggtagcgaggcgggtaccgcgattgcggatgttctgtttggcgactacaacccgagcggcaagctgaccgtgaccttcccgctgcgttttgaggataacccggcgtacctgaacttccagagcaacaaacaagcgtgctggtatggcgaagacgtttacgtgggttatcgttactatgagaccatcgatcgtccggtgctgttcccgtttggtcacggcctgagcttcaccgagttcgattttaccgacatgtttgttcgtctggaggaagagaacctggaagttgaggtggttgtgcgtaacaccggcaagtacgacggtgcggaagtggtgcagctgtatgttgcgccggttagcccgagcctgaaacgtccgatcaaggaactgaaagagtacgcgaaaattttcctggcgagcggtgaagcgaagaccgttcacctgagcgtgccgatcaaatacgcgaccagcttctttgatgagtatcaaaagaaatggtgcagcgaaaagggcgagtataccattctgctgggtagcagcagcgcggacatcaaagttagccaaagcatcaccctggaaaaaaccaccttctggaaaggtctgtaa

SEQ ID NO: 28 - DNA, 1041 bp, Artificial Sequence
  Synthetic
atgaaaagccagctgatctttatggctttggcctcccttgtagcaagtgcaccgctggaacaccagcagcagcatcataaacatgagaaacgcgccgtagttacgcagacagtaactgttgcggcgggccagacagcagcagcgggttccgcccaggcagttgttacctcaagcgcggcgccagcatccgttgcttcaagtgcggccgcgtctgctagctcatcttcttccagctatacctctggcgcttcaggcgatcttagtagtttcaaagatggtactattaaatgttcagaattcccatcaggggatggcgtggtgtccgtctcttggttaggcttcggcggctggtctagtattatgaatctgcagggtggtacttcagagagttgtgagaacggctattattgttcatatgcatgtgaagccggttatagcaaaacacagtggccatctaaccagccgtcagatgggagatcagtgggagggttgctgtgtaaagatggcctgttatatcgctccaatacagcgttcgatacattatgtgtgcctggaaaaggtacagcatccgtggagaataatgtgtctaaaggtatttccatttgtagaacggattatccggggtctgaaaacatgtgcgtcccgacgtgggtcgatgccggtaactcaaacaccttgacagtggtagatgaagataattattatgaatggcagggccttaaaactagtgctcagtattatgtgaataacgccggtgttagtgttgaagatgggtgcatctggggcgatgagtccagcggcgttggaaactgggcgccgttggttttgggggccggttccacggggggtctgacctatctgtctctgattccgaatccaaacaacaaaaaagcaccgaattttaacgtaaaaatcgtggccacggatggaagttcaattaacggagattgcaaatatgaaaatgggatctttgtcggttcttcaaccgatggctgcacggtaactgttacctcaggtagtgcaaaactggttttttattaa

SEQ ID NO: 29 - DNA, 1047 bp, Artificial Sequence
  Synthetic
atgcaggttaaatctattgttaatttactgcttgcctgttccttggctgtggcgcgtccgttggaacacgctcaccatcagcatgataaacgcggcgttgtagtagtaacgaaaaccatcgtcgttgatggtagcacagttgaggctaccgccgctgctcaggtgcaggagcatgcagaaacctttgcagaatcaaccccgtcagccgtcgtttccagttcatccgccccttcatcagcaagctcagcttccgctccagctagttcaggttctttttcagctggtaccaaaggcgtgacatattctccatatcaggccggtggtgggtgtaaaacagcggaagaagtggcatccgatctgtcacagcttaccggttatgaaattattcggctttatggcgtagattgcaaccaggttgagaacgtgtttaaagccaaagcccctggccagaaactttttttgggtatcttttttgtggatgccatcgagtctggcgtatcagctatcgcaagtgccgttaaatcctatggttcttgggatgatgtacacactgtatctgttggcaacgagctggtgaacaatggcgaagccactgttagccagattggacagtatgttagtacggccaaatcagccttacgctctgccggtttcacagggccagtattgtctgttgatacttttattgcagtgattaacaatccggggctgtgtgatttcgcggatgaatatgttgctgtgaacgcccatgcgttcttcgatgggggtattgctgcctcaggggcgggcgattgggcggcagagcagatccagcgcgtctccagtgcgtgcggcgggaaagatgtcttaattgtagaaagcggttggccgtctaaaggagatacgaacggcgccgcagtgccgtcaaaatccaatcagcaggctgcagtccagagtcttggccagaaaattgggagctcatgcattgcctttaacgcatttaatgattattggaaagccgatggtccgttcaacgccgaaaaatattgggggatccttgatagttaa

SEQ ID NO: 30 - DNA, 1395 bp, Artificial Sequence
  Synthetic
atgctgtccacaattctgaatatttttattcttctgttattcatccaggcgtctcttcaggcgcctattccggtggtgaccaaatatgtgaccgaaggtattgccgttgtgactgaaaccaatgtgcgggttgttactaaaaccattccgattgtgcaggtgctgatctccgatggtgcaacctatactcataccctgacgacagtgtcaacggcggaagaaaatggcaacttccagcctattaccacgacatctattgtcaacaaagaagttgtagtaccaacaagcgtaaccccgaatacccagcagacgcgtccgacccaggtagataccacacagaacaatgcggatacaccagcggcgcctacaccatcacctactactagttcaaacaacggcgtgttcaccacatattccacaacacgtagcgtagtcactagtgtagtcgtagtcggaccggatggaagccctattgaaaatactggacagacagcaaaccctactacaactgccccaactacaagcactactgctgcccggaccacaagcagtacgtccaccacacctaccgctagctctacgccaggaggtaatcatccacgtagcatcgtctattctccatattccgatagcagtcagtgtaaagatgcgacaacgatcgaaaccgatcttgagttcattgcctctaaaggcatcagcgcggtacgtatttatggcaatgattgtaactatcttacagttgttttgcctaaatgtgccagtctgggattaaaagtgaatcagggcttttggattggtccaagtggagtagatagcatcgatgatgcagtacaggagtttattcaggcagtcaacggcaacaacggctttaattgggatttattcgaattaattaccgtcggaaacgaagcaatcagtgccggttatgtttcagcgagctccctgatttccaaaattaaagaagtatctagcattctgagctccgcgggttatactggtccaattaccacagccgaaccgcctaacgtatatgaggattatggcgatctgtgctcaaccgatgtaatgtccatcgtgggtgtaaacgcgcattcctattttaataccctttttgcggcctccgattcaggttcatttgtgaaatcacagatcgaagtagtccagaaagcatgctcacgttccgatattactattattgaaaccgggtatccgtcccagggagctaccaatggaaaaaacgttcctagtaaagagaatcagaaaacagcgattttttcaatctttgaggtcgttggaacagatgtaactattcttagtacttatgatgatttgtggaaagatcctggaccgtatgggattgaacagttttttggtgcgatcgatcttttttcttaa